(19) United States
(12) Patent Application Publication
(10) Pub. No.: US 2016/0177407 A1
(43) Pub. Date: Jun. 23, 2016

(54) METHOD FOR DETERMINING DRUG RESISTANCE MUTATIONS IN ANY OF THE NON-STRUCTURAL PROTEIN REGIONS NS3 TO NS5B OF HEPATITIS C VIRUS (HCV) FOR GENOTYPES 1 TO 6

(71) Applicant: VIRCO BVBA, Mechelen (BE)

(72) Inventors: Diana Koletzki, Etterbeek (BE); Jan Martin Berke, Willebroek (BE); Ina Isabel Vandenbroucke, Verrebroek (BE); Leen Roger Vijgen, Herselt (BE)

(21) Appl. No.: 14/579,453

(22) Filed: Dec. 22, 2014

(51) Int. Cl. C12Q 1/70; C07K 14/005

(52) U.S. Cl. CPC C12Q 1/707; C07K 14/005; C12Q 2600/156

(57) ABSTRACT

The present invention relates to a method for determining drug resistance mutations in any of the non-structural protein regions NS3 to NS5B of Hepatitis C Virus (HCV) for genotypes 1 to 6, more in particular for subtype specific genotypes 1a, 1b, 2a, 2b, 3a, 4a and 4d.

This application is the national stage of PCT Application No. PCT/EP2009/062986 filed Oct. 6, 2009, which claims priority from European Patent Application No. 08165949.2, filed Oct. 6, 2008, the entire disclosures of which are hereby incorporated in their entirety.

The present invention relates to a method for determining drug resistance mutations in any of the non-structural protein regions NS3 to NS5B of Hepatitis C Virus (HCV) for genotypes 1 to 6, more in particular for subtype specific genotypes 1a, 1b, 2a, 2b, 3a, 4a and 4d.

HCV is a single stranded, positive-sense RNA virus, with a genome of around 9,600 bases belonging to the Flaviviridae family of viruses in the hepacivirus genus. The NS5B region of the RNA polygene encodes a 65 kDa RNA dependent RNA polymerase (RdRp), which is essential to viral replication. Following the initial acute infection, a majority of infected individuals develop chronic hepatitis because HCV replicates preferentially in hepatocytes but is not directly cytopathic. In particular, the lack of a vigorous T-lymphocyte response and the high propensity of the virus to mutate appear to promote a high rate of chronic infection. Chronic hepatitis can progress to liver fibrosis, leading to cirrhosis, end-stage liver disease, and HCC (hepatocellular carcinoma), making it the leading cause of liver transplantations.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

There are six major HCV genotypes and more than 50 subtypes, which are differently distributed geographically. HCV genotype 1 is the predominant genotype in Europe and the USA. The extensive genetic heterogeneity of HCV has important diagnostic and clinical implications, perhaps explaining difficulties in vaccine development and the lack of response to current therapy.

The genetic variability of HCV complicates amplification, sequencing and genotyping processes. These processes rely on the use of so-called primers complementary to and capable of hybridizing to corresponding nucleic acid sequences of the HCV genome. Due to the high degree of variability of the HCV genome, primers complementary to one HCV species may not be complementary to another species.

To determine the subtype of an HCV clinical isolate an accurate and direct method is sequencing the viral genome in a region that is sufficiently divergent among various species in order to distinguish between HCV genotypes and subtypes accordingly. Phylogenetic analysis of the sequences generated from these regions is used to determine the subtype of clinical isolates.

Several selective and potent antiviral drugs against chronic hepatitis C virus (HCV) infection are currently evaluated in clinical trials. The emergence of drug resistance mutations was proven in previous trials, creating a need for patients to be monitored for the development of such drug-resistance mutations.

In order to improve the identification of HCV types and subtypes for purposes of clinical analysis and therapeutic decision making by a treating physician, there is a continuing need to improve sequencing-based HCV assays.

The hepatitis C virus is, as mentioned above, currently classified into at least 6 major genotypes (FIG. 1). Each genotype differs from the other by 30% to 35% on nucleotide level and may be further divided into several subtypes with sequence diversity typically between 20% and 25% (Simmonds et al., Hepatology 2005; 42(4), 962-973).

The present invention relates to the development of subtype-specific assays for HCV genotype resistance analysis suitable for clinical trial support and regulatory filings.

In more detail the invention relates to genotyping assays covering the complete coding region from NS3 to NS5B as developed on a large panel of clinical samples including protocols for subtypes 1a, 1b, 2a, 2b, 3a, 4a and 4d.

The current invention relates to a NS5B sequence-based subtyping assay detecting all six HCV genotypes and discriminating between the different subtypes.

One aspect of the invention concerns a method for determining drug resistance mutations in any of the non-structural protein regions NS3 to NS5B of Hepatitis C Virus (HCV) for genotypes 1 to 6, more in particular for subtype specific genotypes 1a, 1b, 2a, 2b, 3a, 4a and 4d, present in a sample comprising:
a) obtaining said sample from a patient,
b) extracting viral genetic material from said sample,
c) amplification of the NS5B region of HCV to generate a DNA amplicon of 388 base pairs by using primers having the sequences selected from the group consisting of SEQ ID NO's 1-5,
d) sequencing of the amplicon to obtain a sequence of 329 base pairs by using the sequences selected from the group consisting of SEQ ID NO's 3-5,
e) performing phylogenetic tree analysis using the 329 base pair sequence information of NS5B to obtain HCV-subtype information in said patient sample,
f 1) using subtype-specific primers having the sequences selected from either the group consisting of SEQ ID NO's 6-9, 42-45, 104-107, 120-123, 145-148 or 180-183 for the generation of a DNA amplicon comprising the non-structural protein NS3 (N-terminal 181 amino acids),
g 1) sequencing the NS3 amplicon to obtain a sequence of 543 base pairs by using the sequences selected from the group consisting of SEQ ID NO's 8 and 9; 43 and 45-46; 104 and 106; 120 and 122; 146 and 148 or 180 and 182
or
f 2) using subtype-specific primers having the sequences selected from the group consisting of SEQ ID NO's 13-16, 54 and 59-66, 124-133, 158 and 160-168 or 194-197 for the generation of a DNA amplicon comprising NS5B polymerase,
g 2) sequencing the NS5B polymerase amplicon to obtain a sequence of 1776 base pairs by using the sequences selected from the group consisting of SEQ ID NO's 15-16 and 87-92; 54, 59 and 61-66; 124 and 127-133; 158-159, 161 and 163-168 or 197-204
or
f 3) using subtype-specific primers having the sequences selected from the group consisting of SEQ ID NO's 30-33, 67-70, 93-96, 108-111, 134-137 or 169-172 for the generation of a DNA amplicon comprising NS3 /4A,
g 3) sequencing the NS3/4A protease amplicon to obtain a sequence of 2055 base pairs by using the sequences selected from the group consisting of SEQ ID NO's 34-41; 68 and 71-77; 95 and 97-103; 112-119; 136 and 138-144 or 171 and 173-179
or
f 4) using subtype-specific primers having the sequences selected from the group consisting of SEQ ID NO's 47-50, 78-81, 149-151 and 159 or 184-187 for the generation of a DNA amplicon comprising NS4B/5A,
g 4) sequencing the NS4B/5A amplicon to obtain a sequence of the two genes NS4B and NS5A by using the sequences selected from the group consisting of SEQ ID NO's 51-57; 79 and 81-87; 152-159 or 185 and 187-193;
h) aligning the sequence obtained in step (g 1), (g 2), (g 3) or (g 4) with a reference or wild-type HCV sequence,
i) determining drug resistance mutation(s) in the viral genetic material present in patient sample.

Another embodiment of the current invention is that above method further comprises the steps for performing a NS3 phenotyping assay by
j) generating a NS3 amplicon starting from the DNA amplicon comprising the NS3 (N-terminal 181 amino acids) as obtained in step (f 1) of claim 1 using primers having the sequence of SEQ ID NO 11 and 12,
k) inserting, by InFusion™ cloning or in vitro recombination, said amplicon obtained in step w into a NS3 deleted replication incompetent marker containing shuttle vector having the sequence of SEQ ID NO 10 to obtain a NS3 replication competent recombinant HCV replicon,
l) generating RNA, by in vitro transcription, from said HCV replicon obtained in step (k)
m) transfecting said RNA into suitable cells,
n) determining, based on the expression of the marker gene, the EC₅₀ value and/or fold change as a measure for the presence of drug resistance mutations in a sample.

In another embodiment the invention relates to the above mentioned method further comprising the steps for performing a NS5B phenotyping assay by
o) generating a NS5B amplicon starting from the DNA amplicon comprising the NS5B as obtained in step (f 2) of claim 1 using primers having the sequence of SEQ ID NO 28 and 29,
p) inserting, by in vitro recombination, said amplicon obtained in step (o) into a NS5B deleted replication incompetent marker containing shuttle vector having the sequence of SEQ ID NO 21 or SEQ ID NO 27 to obtain a NS5B replication competent recombinant HCV replicon,
q) generating RNA, by in vitro transcription, from said HCV replicon obtained in step (p)
r) transfecting said RNA into suitable cells,
s) determining, based on the expression of the marker gene, the EC₅₀ value and/or fold change as a measure for the presence of drug resistance mutations in a sample.

Part of the invention is also a vector comprising the HCV genome and a deletion spanning the HCV NS3 N-terminal 181 amino acid region, in particular vector pFK I341 PI luc ΔNS3 7-192_ET (SEQ ID NO.10) and a vector comprising the HCV genome and a deletion spanning the HCV NS5B region, in particular vector pFK_I341_PI_NS3-3_ET_dNS5a/b_5a440-5b591-ScaI (SEQ ID NO 21) and the vector comprising the HCV genome and a deletion spanning the HCV NS5B region, in particular vector pFK_I341_PI_NS3-3_ET_dNS5a/b_5a440-5b591-XbaI (SEQ ID NO 27).

Besides the use of any of the above vectors in any of the methods mentioned, also the primers with SEQ ID NO 1-5 for the amplification of the HCV NS5B region, as obtained from a sample of an HCV-infected patient, belong to the invention.

The use of the primers with SEQ ID NO 1-5 for the preparation of a sequence-based subtyping HCV assay to detect HCV genotypes 1, 2, 3 and 4 and to discriminate between the subtypes 1a, 1b, 2a, 2b, 3a, 4a and 4d, belongs in particular to the current invention.

### EXPLANATION OF FIGURES

FIG. 1: Phylogenetic tree of complete open-reading frame sequences of HCV showing the major 6 genotypes and their most common subtypes. (Simmonds et al. 2005 Hepatology 2005; 42(4), 962-973)

FIG. 2: Overview of amplicons for the integrated HCV platform.

FIG. 3: Development status of the HCV subtyping and subtype-specific genotyping assays and their performance characteristics. Numbers in brackets show the number of tested samples.

FIG. 4: Vector pFK I341 PI luc ΔNS3 7-192_ET (SEQ ID NO.10)

FIG. 5: process overview

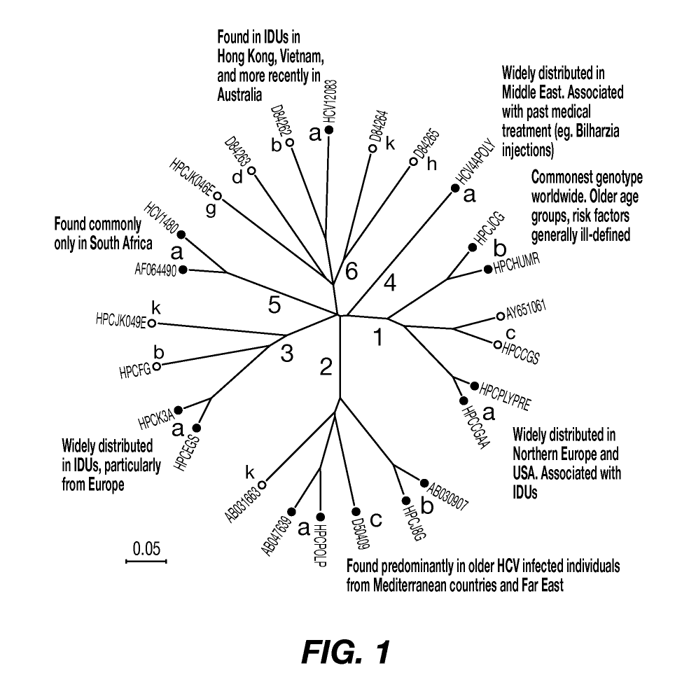

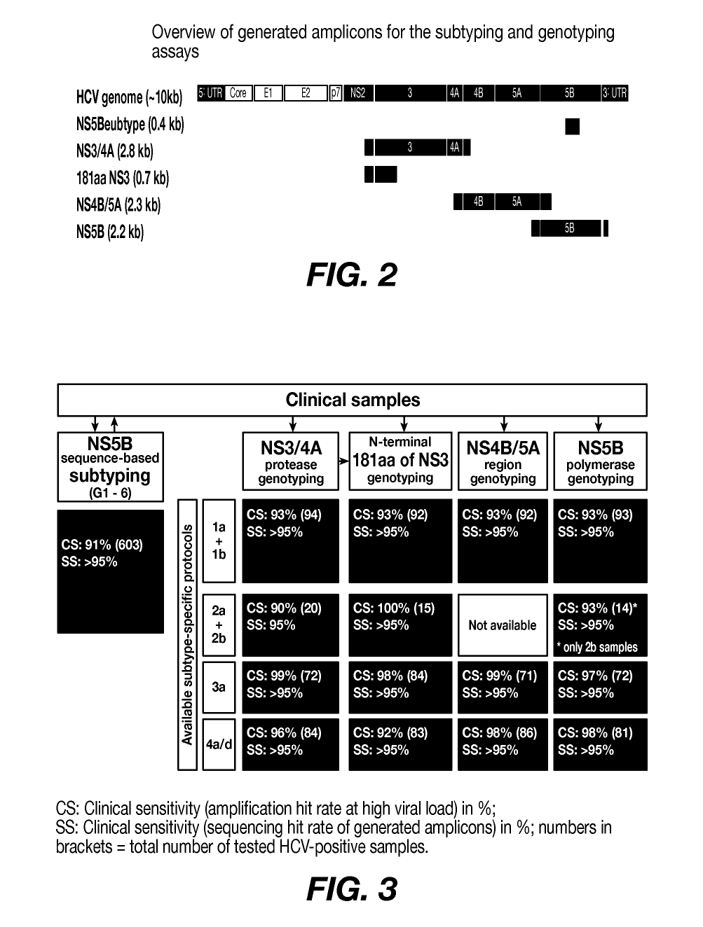

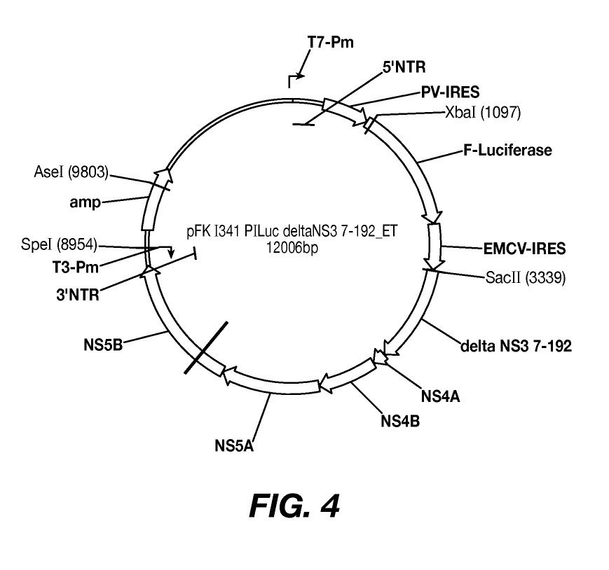

A panel of 603 clinical samples covering all six genotypes (G) was collected. Two test systems were developed: a NS5B sequence-based subtyping assay and a set of subtype-specific genotyping assays to determine drug-resistance mutations in the following target regions: (1) protease inhibitors (complete NS3/4A and the N-terminal 181 as of NS3), (2) polymerase inhibitors (complete NS5B), and (3) others (complete NS4B/5A region). All primer sets have been optimized for subtype specificity and to allow the same PCR protocol to be used for a target region independent of the subtype (FIG. 2). All methods and protocols were optimized and validated to support high-throughput processing of the genotypic resistance assays in a routine operational setting.

The NS5B sequence-based subtyping assay was tested on a set of 603 clinical samples containing all six genotypes with a clinical sensitivity (amplification success rate of high viral load samples) of 91%.

For the subtype-specific genotyping assays, sets of clinical samples of, on average, n=94 for G1a/b, n=16 for G2a/b, n=76 for G3a, and n=83 for G4a/d were tested in the different assays to evaluate the clinical sensitivity. Amplification success rates between 90% and 100% and sequencing success rates between 95% and 100% were achieved (FIG. 3).

### EXAMPLE SECTION

### General Outline

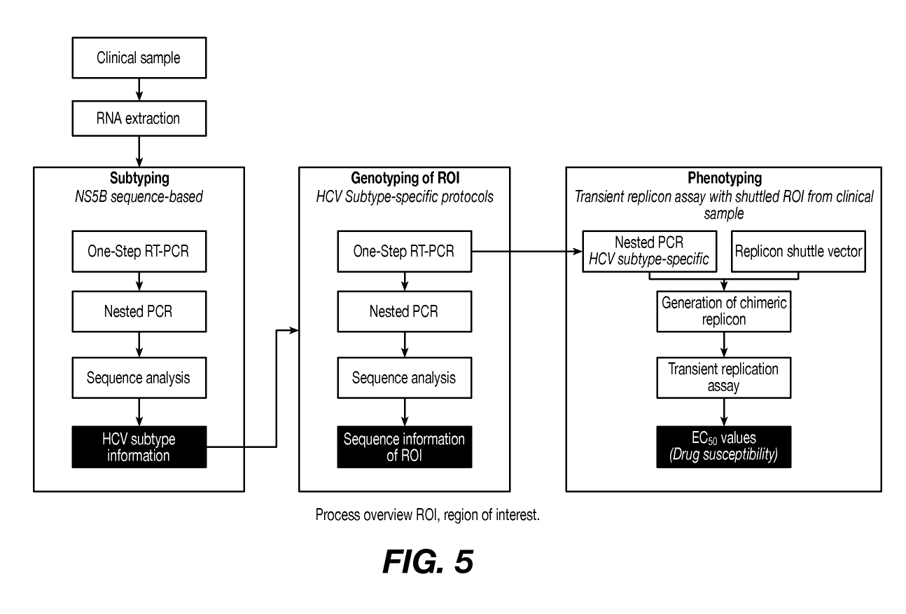

The general process flow is visualized in FIG. 5. It starts with the determination of the HCV subtype of a clinical sample (Subtyping). This subtype information is then used in the subsequent Genotyping process to select the appropriate subtype-specific primers for amplification and sequencing of the target region of interest. The end result of the Genotyping process is the nucleotide and amino acid sequence information of that region. By comparison to a wildtype or viral reference sequence it provides information about the occurrence of amino acid changes. PCR products from the Genotyping process will be used in the Phenotyping process to generate chimeric subgenomic replicons for drug susceptibility assessment. The results of the phenotyping are EC₅₀ values which can be used for interpretation of drug susceptibility (i.e. by calculating EC₅₀ fold change values) of the clinical isolate. Sequence information of the target region and drug susceptibility can be compared.

### 1. Subtyping

An amplicon was generated from patient-derived viral plasma RNA by One-step RT-PCR followed by nested PCR. This amplicon, further referred to as the NS5B subtyping amplicon, contains a 329 bp sequence of the NS5B polymerase domain, which is used for phylogenetic tree analysis in order to obtain subtype information of the clinical isolate. The assay is called the NS5B sequence-based subtyping assay. The subtype information of the clinical isolate will be used in a next step to select the appropriate subtype-specific amplification and sequencing primers in order to obtain sequence information of the region of interest in the genotyping assay.

### 2. Genotyping

Using subtype-specific primers, an amplicon of the NS3 protease domain (containing the catalytic domain) is generated from patient-derived viral plasma RNA by One-Step RT-PCR followed by nested PCR. This amplicon, further referred to as the NS3 amplicon, contains the catalytic domain of the NS3 protease. This amplicon is going to be sequenced with subtype-specific sequencing primers in the HCV NS3 protease genotypic assay.

Using subtype-specific primers, an amplicon of the complete NS5B polymerase can also be generated from patient-derived viral plasma RNA by One-Step RT-PCR followed by nested PCR. This amplicon, further referred to as the NS5B amplicon, contains the complete NS5B gene. This amplicon is going to be sequenced with subtype-specific sequencing primers in the HCV NS5B polymerase genotypic assay.

The same can be achieved using subtype-specific primers for other dedicated HCV regions like NS3/NS4A or NS4B/NS5A and the like.

### 3. Phenotyping

A NS3-deleted replication incompetent shuttle vector, further referred to as the delta[NS3] backbone, has been generated based on the subgenomic replicon con1b sequence. The NS3 amplicon is generated, from patient-derived material, replicon plasmid DNA, synthetic genes or PCR products of replicon RNA, by PCR using the One-Step RT-PCR product of the HCV NS3 protease genotypic assay. In-Fusion™ cloning (Clontech) of the PCR-generated NS3 amplicon and the delta[NS3] backbone resulted in a replication-competent recombinant HCV replicon that was used in experiments to evaluate HCV NS3 phenotypic drug resistance.

A NS5B-deleted replication incompetent shuttle vector, further referred to as the delta[NS5B] backbone has been generated based on the subgenomic replicon con1b sequence. The NS5B amplicon is generated, from patient-derived material, replicon plasmid DNA, synthetic genes or PCR products of replicon RNA, by PCR using the One-Step RT-PCR product of the HCV NS5B polymerase genotypic assay. In vitro cloning (using BD In-Fusion™, Clontech Laboratories Inc.) of the PCR-generated NS5B amplicon and the delta[NS5B] backbone resulted in a replication-competent recombinant HCV replicon that was used in experiments to evaluate HCV NS5B phenotypic drug resistance.

### Example 1

### NS5B Sequence-Based Subtyping Assay

### A. RNA-Extraction

From a total 500 μl of plasma, total RNA was extracted using the EasyMAG™ RNA extraction platform (BioMerieux). After elution in 60 μl elution buffer, RNA was stored at −80° C. until use for amplicon generation.

### B. One-Step RT-PCR

Five μl RNA were mixed with 2× reaction buffer, 120 ng/ml yeast tRNA (Ambion Inc., Woodward, USA), 0.2 μM primer NS5Bsubtype_A (TGGGGTTCGCGTATGATACCCGCTGCTTTGA) (SEQ ID NO: 1), 0.2 μM primer NS5Bsubtype_B (TGGGGTTTTCTTACGACACCAGGTGCTTTGA) (SEQ ID No: 2), 0.2 μM primer Pr2 (published in Sandres-Saunes et al. 2003) and 0.5 μl of the Superscript™ III RT/Platinum Taq High Fidelity enzyme mix from the SuperScript™ III One-Step RT-PCR System (Invitrogen) in a total volume of 25 μl. The cDNA synthesis is performed for 30 min at 52° C. followed by a denaturation step at 94° C. for 2 min. Thermal cycling consisted of 50 cycles of denaturation at 94° C. for 15 s, annealing at 63° C. for 30 s and elongation at 72° C. for 30 s. Final extension took place at 72° C. for 5 min. An aliquot of the resulting amplification product was used for a nested PCR step.

### C. Inner PCR

For the nested PCR, 2.5 μl from the One-Step RT-PCR product were mixed with 10× buffer 2 from the Expand™ High Fidelity kit (Roche), 0.35 mM dNTPs (Promega), 0.4 μM primer NS5Bsubtype_C (CCGTATGATACCCGCTGCTTTGACTCAAC) (SEQ ID NO: 3), 0.3 μM primer NS5Bsubtype_D (TCCTACGACACCAGGTGCTTTGATTCAAC) (SEQ ID NO: 4), 0.4 μM primer NS5Bsubtype_E (AATTCCTGGTCATAGCCTCCGTGAAGACTC) (SEQ ID NO: 5) and 0.075 U/μl of DNA polymerase (Roche, Basel, Switzerland) to give a total volume of 50 μl. Initial denaturation was 94° C. for 2 min and thermal cycling consisted of 30 cycles of denaturation at 94° C. for 15 s, annealing at 56° C. for 30 s and elongation at 72° C. for 30 s. Final extension took place at 72° C. for 5 min. The amplicons were purified using the QIAQuick 96 PCR purification kit (Qiagen,). Final volume of purified amplicons was 100 μl.

### D. Raw Sequence Analysis

Sequencing reaction was performed according to standard procedures by using the primers from the nested PCR for sequencing of both directions, forward and reverse. Electropherograms were retrieved from the ABI3730 capillary sequencer and imported into Seqscape v2.5 (Applied Biosystems). Sequence ends were trimmed based on quality values and the 329 bp length of the subtyping reference sequence; the latter spanned the regions between the amplification primers. No insertions, deletions or STOP codons were allowed to occur in the sequences.

### E. Phylogenetic Tree Analysis

The sample sequences with a length of 329 bp were merged with subtype reference sequences in BioEdit ((Ibis Therapeutics; public source internet: www.mbio.ncsu.edu/BioEdit/bioedit.html) and subsequently analysed in MEGA v3.1 (public source, internet: http://www.megasoftware.net/) using Neighbour-Joining tree and Jukes-Cantor distance model.

### Results:

NS5B Subtyping Sequences from HCV-1b Infected Patients:

>Pt 1 NS5B subtyping
AGTCACCGAGAATGATATCCGTGTTGAGGAGTCAATTTACCAATGCTGTG
ACTTGGCCCCCGAAGCCAAACAGGCCATAAGGTCGCTCACAGAGCGGCTT
TAYATCGGGGGTCCCCTGACTAATTCAAAAGGGCAGAACTGCGGTTATCG
CCGGTGCCGCGCGAGCGGCGTGCTGACGACCAGCTGCGGTAATACCCTC
ACCTGTTACTTGAAGGCCACCGCGGCCTGTCGAGCTGCAAAGCTCCAGG
ACTGCACGATGCTCGTGTGCGGGGACGACCTTGTCGTTATCTGTGAAAGC
GCGGGAACCCAAGAGGACGCGGCGAACCTAC
>Pt 2 NS5B subtyping
TGTCACYGAGAGTGACATCCGYGTTGAGGAGTCAATCTACCAATGTTGTG
ACTTGGCCCCCGAAGCCAGACAGGCCATAAAGTCGCTCACAGAGCGGCT
TTAYATCGGGGGTCCCCTGACTAAYTCAAAAGGRCAGAACTGCGGYTATC
GCCGGTGCCGCGCGAGCGGCGTGCTGACGACTAGCTGCGGYAACACCC
TCACMTGTTACYTGAAGGCCTCTGCAGCCTGTCGAGCTGCRAAGCTCCAG
GACTGCACGATGCTCGTGTGCGGGGACGACCTTGTCGTTATCTGCGAGA
GTGCTGGGACCCAGGAGGACGYGGCGAGCCTAC
>Pt 3 NS5B subtyping
GGTCACTGAGAATGACATTCGTGTCGAGGAGTCGATCTACCAATGCTGTG
ACTTGGCCCCCGAAGCCAGACARGCCATAAGGTCGCTCACGGAGCGGCT
TTATATCGGGGGTCCCCTGACTAATTCAAAAGGGCAGAACTGCGGTTATC
GCCGGTGCCGCGCGAGCGGTGTACTGACGACCAGCTGTGGTAATACCCT
CACATGTTACTTGAAGGCCTCTGCGGCCTGTCGAGCTGCCAAGCTCCAGG
ACTGCACGATGCTCGTGAACGGAGACGACCTTGTCGTTATCTGTGAGAGC
GCGGGAACCCAARAGGACGCAGCGAACCTAC
>Pt 4 NS5B subtyping
RGTCACCGAGAGKGACATCCGTGTTGAGGAGTCRATYTACCAATGTTGTG
ACTTGGCCCCCGAAGCCAGACAGGCCATAAAGTCGCTCACRGAGCGGCT
CTATATCGGGGGCCCCCTGACTAATTCAAAAGGGCAGAACTGCGGTTATC
GCCGGTGCCGCGCCAGCGGCGTRCTGACGACCAGCTGCGGTAATACCCT
CACATGTTACTTGAAGGCCTCTGCGGCCTGTCGAGCTGCAAAGCTCCAGG
ACTGCACGATGCTTGTGTGYGGAGACGACCTYGTCGTTATCTGTGAGAGC
GCGGGGACCCAGGAGGACGCGGCGAGCCTAC
>Pt 5 NS5B subtyping
GGTCACTGAGAGTGAYATCCGTGTYGAGGAGTCAATATACCAATGTTGTG
ACTTGGCCCCCGAAGCCAGACAGGCCATAAAGTCGCTCACAGAGCGGCT
CTATGTTGGGGGTCCCCTGACTAAYTCAAAAGGGCAGAACTGCGGTTATC
GCCGGTGCCGCGCCAGCGGCGTGCTGACGACCAGCTGCGGTAATACCCT
CACTTGTTACTTGAAAGCCTCTGCRGCCTGTCGAGCTGCGAAGCTCCAGG
ACTGCACGATGCTCGTGTGTGGAGACGACCTTGTCGTTATCTGCGAAAGC
GCGGGAACCCAGGAGGACGCGGCGAGCCTAC
>Pt 12 NS5B subtyping
AGTCACTGAGAGTGACATCCGCGTTGAGGAGTCAATCTACCAATGTTGTG
ACTTGGCCCCCGAAGCCAAACAGGCCATAAAGTCGCTCACAGAGCGGCT
TTACATCGGGGGTCCCCTGACTAATTCAAAAGGGCAGAACTGCGGCTATC
GCCGGTGCCGCGCCAGCGGCGTACTGACGACCAGCTGTGGTAATACCCT
CACATGTTACTTGAAAGCCTCTGCGGCCTGTCGAGCTGCAAAGCTCCAGG
ACTGCACGATGCTCGTGTGCGGAGACGACCTTGTCGTTATCTGTGAGAGC
GCGGGAACCCAGGAGGACGCGGCGAGCCTAC
>Pt 13 NS5B subtyping
GGTCACTGAGAGTGATATCCGTACTGAGGAGTCTATTTACCAATGTTGTG
ACCTGGCCCCCGAAGCTAGACAAGTCATAAGGTCGCTCACAGAGCGGCTT
TAYATYGGGGGCCCCCTGACYAATTCAAAAGGGCAGAACTGCGGTTATCG
CCGGTGCCGYGCGAGCGGCGTGCTGACGACTAGCTGCGGTAATACCCTCA
CATGTTACTTGAAGGCCTCTGCGGCCTGTCGAGCTGCAAAGCTCCGGGA
CTGCACGATGCTCGTGTGCGGAGACGACCTCGTCGTTATCTGTGAAAGCG
CGGGGACCCAGGAGGACGCGGCTAGCCTAC
>Pt 14 NS5B subtyping
AGTCACCGAGAATGATATCCGTGTTGAGGAGTCAATTTACCAATGCTGTG
ACTTGGCCCCCGAAGCCAAACAGGCCATAAGGTCGCTCACAGAGCGGCTT
TAYATCGGGGGTCCCCTGACTAATTCAAAAGGGCAGAACTGCGGTTATCG
CCGGTGCCGCGCGAGCGGCGTGCTGACGACCAGCTGCGGTAATACCCTC
ACCTGTTACTTGAAGGCCACCGCGGCCTGTCGAGCTGCAAAGCTCCAGG
ACTGCACGATGCTCGTGTGCGGGGACGACCTTGTCGTTATCTGTGAAAGC
GCGGGAACCCAAGAGGACGCGGCGAACCTAC
>Pt 15 NS5B subtyping
GGTCACYGAGAGYGACATCCGTACTGAGGAGTCAATTTACCAATGTTGTG
ACTTGGCCCCCGAAGCCAGACAGGTTATAAGGTCGCTCACAGAGCGGCT
TTATATCGGGGGTCCTYTGACTAATTCAAAAGGGCAGAACTGCGGCTATC
GCCGGTGTCGCGCAAGCGGCGTGCTGACGACCAGCTGCGGCAATACCCT
CACATGTTACCTGAAGGCCACTGCAGCCTGTCGAGCTGCGAAGCTCCAG
GACTGCACAATGCTTGTGTGTGGGGACGACCTTGTCGTYATCTGTGAGAG
CGCGGGGACCCAAGAGGACGCAGCGAGCCTAC
>Pt 16 NS5B subtyping
GGTCACTGAGAATGACATYCGTGTTGAGGAGTCAATTTACCAATGTTGTG
ACTTGGCYCCCGAAGCCAGACAGGYCATAAGGTCGCTCACAGAGCGGCTT
TAYATCGGGGGTCCYCTAACCAATTCAAAAGGGCAAAACTGCGGTTATCG
CCGGTGTCGCGCRAGCGGCGTGCTGACGACTAGCTGCGGCAAYACCCTT
ACATGTTACTTGAARGCCTCTGCRGCCTGTCGAGCTGCGAAGCTCCAGGA
CTGCACGATGCTCGTGTGCGGAGACGACCTCGTCGTTATCTGTGAGAGC
GCGGGGACCCACGAGGATGCGGCGAGCCTAC

These sequences were subtyped using phylogenetic analysis. Table 1 shows the result.

TABLE 1

| | NS5B sequence based subtype |
|---|---|
| Sample ID | information after phylogenetic analysis |
| Pt 1 | 1b |
| Pt 2 | 1b |
| Pt 3 | 1b |
| Pt 4 | 1b |
| Pt 5 | 1b |
| Pt 12 | 1b |
| Pt 13 | 1b |
| Pt 14 | 1b |
| Pt 15 | 1b |
| Pt 16 | 1b |

Based on the NS5B sequence-based subtype information, the appropriate subtype-specific primers were selected for the amplification of the NS3 protease domain.

### Example 2

### HCV NS3 Genotyping Assay

### A. One-Step RT-PCR

Five μl RNA were mixed with 2× reaction buffer, 120 ng/ml yeast tRNA (Ambion), 0.2 μM forward primer 1b-NS3_out_F

(GCGTGTGGGGACATCATCTTAGG) (SEQ ID NO: 6), 0.2 μM primer 1b_NS3_out_R (GCTGCCAGTGGGAGCGTG) (SEQ ID NO: 7) and 0.5 μl of the Superscript™ III RT/Platinum Taq High Fidelity enzyme mix from the SuperScript™ III One-Step RT-PCR System (Invitrogen) in a total volume of 25 μl. The cDNA synthesis is performed for 30 min at 52° C. followed by a denaturation step at 94° C. for 2 min. Thermal cycling consisted of 50 cycles of denaturation at 94° C. for 15 s, annealing at 58° C. for 30 s and elongation at 72° C. for 1 min. Final extension took place at 72° C. for 5 min. An aliquot of the resulting amplification product was used for a nested PCR step.

### B. Inner PCR

For the nested PCR, 2.5 μl from the One-Step RT-PCR product was mixed with 10× buffer 2 from the Expand™ High Fidelity kit (Roche), 0.35 mM dNTPs (Promega), 0.4 μM primer 1b_NS3_in_F (TCATCTTAGGCCTGCCCGTCTC) (SEQ ID NO: 8), 0.4 μM primer 1b_NS3_in_R (GGGAGCGTGTAGATGGGCCAC) (SEQ ID NO: 9) and 0.075 U/μl of Expand™ High Fidelity DNA polymerase (Roche) to give a total volume of 50 μl. Initial denaturation was 94° C. for 2 min and thermal cycling consisted of 30 cycles of denaturation at 94° C. for 15 s, annealing at 58° C. for 30 s and elongation at 72° C. for 1 min. Final extension took place at 72° C. for 5 min. The amplicons were purified using the QIAQuick 96 PCR purification kit (Qiagen). Final volume of purified amplicons was 100 μl.

### C. Raw Sequence Analysis

Sequencing reaction was performed according to standard procedures by using the primers from the nested PCR for sequencing of both directions, forward and reverse (SEQ ID No's 8-9). Electropherograms were retrieved from the ABI3730 capillary sequencer and imported into Seqscape v2.5 (Applied Biosystems). Sequence ends were trimmed based on quality values and the 543 bp (coding sequence for the N-terminal 181 aa of NS3) length of the subtyping reference sequence; the latter spanned the regions between the amplification primers. No insertions, deletions or STOP codons were allowed to occur in the sequences.

### Result:

NS3 Protease Sequences from Five (5) HCV-1b Patient Isolates

>Pt 1 NS3
GCGCCTATCACGGCCTACGCCCARCAAACACGGGGCTTGTTTGGCTGTAT
CATCACTAGCCTCACAGGCCGGGACAAGAACCAGGTCGAGGGGGAGGTC
CAAGTGGTTTCCACCGCCACACAATCTTTCCTGGCGACCTGTGTCAACGG
TGTKTGTTGGACTGTCTTCCACGGCGCCGGTTCAAAGACCCTGGCTGGCC
CAAAGGGYCCAATCACCCAAATGTACACCAATGTAGACCAGGACCTCGTC
GGCTGGCCGGCCCCCCCYGGGGCGCGCTCTCTRACACCATGCACCTGTG
GCAGCTCGGACCTTTACTTGGTCACGAGGCATGCTGATGTTATCCCGGTG
CGCCGGCGGGGCGACAGTAGGGGRAGCCTACTCTCCCCCAGGCCTGTG
TCCTACTTAAAAGGCTCTTCGGGTGGWCCRCTGCTCTGCCCCTCGGGGC
ACGCTGTGGGCGTCTTCCGGGCTGCTGTGTGCACCCGGGGGGTCGCGA
AGGCGGTGGACTTTGTACCCGTAGAGTCTATGGAGACTACCATGCGGTCC
>Pt 2 NS3
GCGCCCATCACGGCCTACGCCCAACARACGAGGGGCCTACTTGGCTGTA
TCATCACCAGCCTCACAGGCCGGGACAAGAACCAGGTYGAGGGGGAGGT
TCAGGTGGTCTCCACTGCAACACAGTCCTTCCTGGCRACTTGCATCAACG
GCGTGTGTTGGACTGTCTTTCATGGAGCCGGCTCTAAGACCCTAGCCGGC
CCAAAGGGGCCGATCACCCAGATGTACACCAATGTAGACCAGGACCTCGT
CGGCTGGCAAGCGCCCCCYGGGGCGCGTTCCTTGACACCGTGCACCTGC
GGCAGCTCGGACCTTTACTTGGTCACGAGGCATGCCGATGTCATTCCGGT
GCGCCGGCGAGGTGACAGCAGGGGGAGCTTGCTCTCCCCCCGGCCCAT
TTCYTACTTRAAAGGCTCTTCGGGTGGTCCRYTGCTCTGCCCCTCGGGGC
ACGCYGTGGGCATCTTCCGGGCTGCCGTGTGCACYCGGGGGGTTGCCAA
GGCRGTGGATTTTGTACCCGTTGAGTCTATGGAAACTACYATGCGGTCC
>Pt 3 NS3
GCGCCTATTACGGCCTACGCCCAACAGACGAGGGGCCTATTAGGCTGCA
TCATCACTAGCCTCACAGGCCGAGACAAGAACCAGGTCGAGGGGGAGGT
TCAGGTGGTTTCTACCGCAACACAATCCTTCCTAGCGACTTGCGTCAACG
GCGTGTGTTGGACTGTCTATCATGGCGCCGGCTCTAAGACCTTAGCCGGC
CCAAAGGGGCCTGTCACCCAAATGTACACCAATGTAGACCAAGACCTCGT
CGGCTGGCCAGCGCCCCCCGGGGCGCGTTCCTTGACACCATGTACTTGC
GGCAGTTCGGACCTTTACTTGGTCACGAGACATGCCGATGTCATTCCGGT
GCGCCGGCGGGGCGACAGCAGGGGGAGCCTGCTCTCCCCCAGGCCTGT
CTCCTATTTGAAGGGCTCTTCGGGTGGTCCACTGCTCTGCCCTTCAGGGC
ACGCCGTGGGCATCTTCCGGGCTGCCGTGTGCACCCGAGGGGTTGCCAA
GGCGGTGGACTTTGTGCCCGTCGAGTCCATGGAAACTACTATGCGGTCT
>Pt 4 NS3
GCGCCTATCACGGCTTACTCCCAACAGACGCGGGGCCTGCTTGGCTGCA
TCATCACYAGCCTCACAGGCAGRGACAAGAACCAGGTCGAGGGGGAAGT
CCAAGTGGTTTCCACCGCAACACAATCTTTTCTAGCGACCTGTGTCAACG
GCGTGTGTTGGACTGTTTTCCATGGCGCCGGCTCAAAAACCTTAGCCGGC
CCAAAGGGCCCGGTCACCCAAATGTACACCAATGTAGACCAGGACCTCGT
CGGCTGGCAGGCGCCTACCGGGGCGCGTTCTTTAACACCATGCACCTGC
GGCAGCTCGGACCTTTATTTGGTCACGAGGCATGCTGATGTCATTCCGGT
GCGCCGGCGGGGCGACAGCCGGGGGAGTCTACTCTCCCCCAGGCCCGT
CTCCTACTTGAAGGGCTCCTCGGGTGGTCCGCTGCTCTGCCCCTCGGGG
CATGCAGTGGGCATCTTCCGGGCTGCCGTGTGCACCCGGGGGGTCGCAA
AGGCAGTGGACTTCATACCCGTTGAGTCTATGGAAACTACTATGCGGTCC
>Pt 5 NS3
GCGCCTATCACAGCCTACTCCCAACAGACGCGGGGCCTGCTTGGCTGCA
TCATCACTAGCCTCACAGGCCGGGACAAGAACCAGGTCGAGGGGGAGGT
TCAAGTGGTTTCCACCGCGACACAATCTTTCCTGGCGACCTGCGTCAACG
GCGTGTGTTGGACTGTCTACCATGGTGCCGGCTCGAAGACCCTAGCCGG
CCCAAAGGGCCCGATCACCCAAATGTACACCAATGTAGACCAGGACCTCG
TCGGCTGGCCGGCGCCCTCCGGAGCGCGCTCCTTGACACCGTGCACCTG
CGGCAGCTCAGACCTYTACTTGGTCACGAGGCATGCTGATGTTGTTCCGG
TGCGCCGGCGGGGCGACAGCAGGGGAAGCCTACTCTCCCCCAGGCCCA
TTTCCTACTTGAAGGGCTCTTCGGGTGGCCCGCTGCTTTGCCCCTCGGGG
CACGCGGTGGGCATCTTCCGGGCTGCTGTATGCACCCGGGGGGTCGCGA
AGGCGGTGGACTTTGTACCCGTTGAGTCTATGGAAACCACCATGCGGTCT

D. Alignment of Sequences with Reference Sequence

The alignment shows the nucleotide sequence of the NS3 protease domain of an HCV-1b isolate from an untreated patient. The sequences were aligned against a reference sequence. Homologies between the two sequences are plotted as dots.

| | |
|---|---|
| | 10 20 30 40 |
| | ....\|....\|....\|....\|....\|....\|....\|....\| |
| con1b reference | gcgcctattacggcctactcccaacagacgcgaggcctac |
| Pt 5 | ........C..A....................G.....G. |
| | 50 60 70 80 |
| | ....\|....\|....\|....\|....\|....\|....\|....\| |
| con1b reference | ttggctgcatcatcactagcctcacaggccgggacaggaa |
| Pt 5 | ....................................A... |
| | 90 100 110 120 |
| | ....\|....\|....\|....\|....\|....\|....\|....\| |
| con1b reference | ccaggtcgagggggaggtccaagtggtctccaccgcaaca |
| Pt 5 | ..................T........T........G... |
| | 130 140 150 160 |
| | ....\|....\|....\|....\|....\|....\|....\|....\| |
| con1b reference | caatctttcctggcgacctgcgtcaatggcgtgtgttgga |
| Pt 5 | ..........................C............. |
| | 170 180 190 200 |
| | ....\|....\|....\|....\|....\|....\|....\|....\| |
| con1b reference | ctgtctatcatggtgccggctcaaagacccttgccggccc |
| Pt 5 | .......C..............G........A........ |
| | 210 220 230 240 |
| | ....\|....\|....\|....\|....\|....\|....\|....\| |
| con1b reference | aaagggcccaatcacccaaatgtacaccaatgtggaccag |
| Pt 5 | .........G.......................A...... |
| | 250 260 270 280 |
| | ....\|....\|....\|....\|....\|....\|....\|....\| |
| con1b reference | gacctcgtcggctggcaagcgccccccggggcgcgttcct |
| Pt 5 | ................CG......T....A.....C.... |
| | 290 300 310 320 |
| | ....\|....\|....\|....\|....\|....\|....\|....\| |
| con1b reference | tgacaccatgcacctgcggcagctcggacctttacttggt |
| Pt 5 | .......G.................A.....Y........ |
| | 330 340 350 360 |
| | ....\|....\|....\|....\|....\|....\|....\|....\| |
| con1b reference | cacgaggcatgccgatgtcattccggtgcgccggcggggc |
| Pt 5 | ............T.....TG.................... |
| | 370 380 390 400 |
| | ....\|....\|....\|....\|....\|....\|....\|....\| |
| con1b reference | gacagcagggggagcctactctcccccaggcccgtctcct |
| Pt 5 | ...........A.....................A.T.... |
| | 410 420 430 440 |
| | ....\|....\|....\|....\|....\|....\|....\|....\| |
| con1b reference | acttgaagggctcttcgggcggtccactgctctgcccctc |
| Pt 5 | ...................T..C..G.....T........ |
| | 450 460 470 480 |
| | ....\|....\|....\|....\|....\|....\|....\|....\| |
| con1b reference | ggggcacgctgtgggcatctttcgggctgccgtgtgcacc |
| Pt 5 | .........G...........C........T..A...... |
| | 490 500 510 520 |
| | ....\|....\|....\|....\|....\|....\|....\|....\| |
| con1b reference | cgaggggttgcgaaggcggtggactttgtacccgtcgagt |
| Pt 5 | ..G.....C..........................T.... |
| | 530 540 |
| | ....\|....\|....\|....\|... |
| con1b reference | ctatggaaaccactatgcggtcc |
| Pt 5 | .............C........T |

The following shows the amino acid sequence of the NS3 protease domain of an HCV-1b isolate from an untreated patient. The sequences were aligned against a reference sequence. Homologies between the two sequences are plotted as dots.

| | |
|---|---|
| | 10 20 30 40 |
| | ....\|....\|....\|....\|....\|....\|....\|....\| |
| con1b reference | APITAYSQQTRGLLGCIITSLTGRDRNQVEGEVQVVSTAT |
| Pt 5 | .........................K.............. |
| | 50 60 70 80 |
| | ....\|....\|....\|....\|....\|....\|....\|....\| |
| con1b reference | QSFLATCVNGVCWTVYHGAGSKTLAGPKGPITQMYTNVDQ |
| Pt 5 | ........................................ |
| | 90 100 110 120 |
| | ....\|....\|....\|....\|....\|....\|....\|....\| |
| con1b reference | DLVGWQAPPGARSLTPCTCGSSDLYLVTRHADVIPVRRRG |
| Pt 5 | .....P..S..............X.........V...... |
| | 130 140 150 160 |
| | ....\|....\|....\|....\|....\|....\|....\|....\| |
| con1b reference | DSRGSLLSPRPVSYLKGSSGGPLLCPSGHAVGIFRAAVCT |
| Pt 5 | ...........\|............................ |
| | 170 180 |
| | ....\|....\|....\|....\|. |
| con1b reference | RGVAKAVDFVPVESMETTMRS |
| Pt 5 | ..................... |

NS3 amplicons from these five HCV-1b isolates were further used in the NS3 replicon phenotyping assay.

### HCV NS5B Polymerase Genotyping Assay

### One-Step RT-PCR:

Five μl RNA were mixed with 2× reaction buffer, 120 ng/ml yeast tRNA (Ambion Inc.), 0.2 μM primer 1b_NS5B_out_F (TAGAGTCCTGGAAGGACCCGG) (Sequence ID NO:13), 0.2 μM primer 1b_NS5B_out_R (GGCCTGGAGTGGTTAGCTCCCC) (Sequence ID NO:14) and 0.5 μl of the Superscript™ III RT/Platinum Taq High Fidelity enzyme mix from the SuperScript™ III One-Step RT-PCR System (Invitrogen) in a total volume of 25 μl. The cDNA synthesis is performed for 30 min at 47° C. followed by a denaturation step at 94° C. for 2 min. Thermal cycling consisted of 50 cycles of denaturation at 94° C. for 15 s, annealing at 59° C. for 30 s and elongation at 68-° C. for 2 min 30 s. Final extension took place at 68° C. for 5 min. An aliquot of the resulting amplification product was used for a nested PCR step.

### Inner PCR:

For the nested PCR, 2.5 μl from the One-Step RT-PCR product was mixed with 10× buffer 1 from the Expand™ Long Template High Fidelity kit (Roche, Basel, Switzerland), 0.35 mM dNTPs (Promega), 0.4 μM primer 1b_NS5B_in_F (TGGAAGGACCCGGACTACG) (Sequence ID NO:15), 0.4 μM primer 1b_NS5B_in_R (GAGTGGTTAGCTCCCCGTTCA) (Sequence ID NO:16) and 0.075 U/μl of Expand™ High Fidelity DNA polymerase (Roche,) to give a total volume of 50 μl. Initial denaturation was 94° C. for 2 min and thermal cycling consisted of 30 cycles of denaturation at 94° C. for 15 s, annealing at 59° C. for 30 s and elongation at 68° C. for 2 min 30 s. Final extension took place at 68° C. for 5 min. The amplicons were purified using the QIAQuick 96 PCR purification kit (Qiagen). Final volume of purified amplicons was 100 μl.

### Raw Sequence Analysis

Sequencing reaction was performed according to standard procedures by using 8 sequencing primers (SEQ ID No's 15-16 and 87-92) to cover both directions, forward and reverse. Electropherograms were retrieved from the ABI3730 capillary sequencer and imported into Seqscape v2.5 (Applied Biosystems). Sequence ends were trimmed based on quality values and the 1776 bp (coding sequence of the NS5B polymerase) length of the subtype-specific reference sequence; the latter spanned the regions between the amplification primers. No insertions, deletions or STOP codons were allowed to occur in within the sequences.

### Result:

NS5B Polymerase Sequences from Five HCV-1b Clinical Isolates

>Pt 12 NS5B
TCGATGTCCTACACGTGGACGGGCGCCCTGATCACGCCGTGCGCCGCGG
AGGAAAGCAAGCTGCCTATCAATGCATTGAGCAACTCACTGCTGCGTCAC
CACAATATGGTTTATGCTACAACATCCCGCAGCGCAAGCCAGCGGCAGAA
GAAGGTCACTTTTGACAGACTGCAAGTCCTGGACGACCACTACCGGGACG
TGCTCAAGGAGATGAAGGCGAAGGCGTCCACAGTTAAGGCTAAGCTTCTA
TCTGTAGAGGAAGCCTGTAAACTGACGCCCCCACATTCGGCCAGATCCAA
ATTTGGCTAYGGGGCAAAGGACGTCCGGAACCTATCCAGCAAGGCCGTTA
ACCACATCCGCTCCGTGTGGAAGGACTTGCTGGAAGACACTGAGACACCA
ATTGACACCACCATCATGGCAAAAAACGAGGTYTTCTGCGTCCAACCAGA
GAAAGGAGGCCGCAAGCCAGCTCGCCTTATCGTGTTCCCAGACTTGGGA
GTTCGTGTGTGCGAGAAAATGGCCCTTTACGACGTGGTCTCCACTCTTCC
TCAAGCCGTGATGGGCTCCTCATATGGATTCCAGTACTCTCCTGGACAGC
GGGTTGAATTCCTGGTGAATGCCTGGAAGTCGAAGAAGAACCCTATGGGC
TTCGCATATGACACCCGCTGTTTTGACTCAACAGTCACTGAGAGTGACAT
CCGCGTTGAGGAGTCAATCTACCAATGTTGTGACTTGGCCCCCGAAGCCA
AACAGGCCATAAAGTCGCTCACAGAGCGGCTTTACATCGGGGGTCCCCTG
ACTAATTCAAAAGGGCAGAACTGCGGCTATCGCCGGTGCCGCGCCAGCG
GCGTACTGACGACCAGCTGTGGTAATACCCTCACATGTTACTTGAAAGCC
TCTGCGGCCTGTCGAGCTGCAAAGCTCCAGGACTGCACGATGCTCGTGT
GCGGAGACGACCTTGTCGTTATCTGTGAGAGCGCGGGAACCCAGGAGGA
CGCGGCGAGCCTACGAGTCTTCACGGAGGCTATGACTAGGTACTCCGCC
CCCCCCGGGGACCCGCCCCAGCCAGAGTACGACTTGGAGTTGATAACAT
CATGCTCCTCCAACGTGTCGGTCGCGCACGATGCATCCGGCAAACGGGT
GTATTACCTCACCCGTGACCCCACCACCCCCCTCGCGAGGGCTGCGTGG
GAAACAGCTAGACACACTCCAGTTAATTCTTGGCTAGGCAACATCATTAT
GTATGCGCCCACCCTGTGGGCAAGGATGATTTTGATGACTCACTTCTTCT
CCATCCTTCTAGCTCAAGAACAACTTGAAAAAGCCCTGGATTGTCAGATC
TACGGGGCCTGCTACTCCATTGAGCCACTTGACCTACCTCAGATCATTCA
RCGACTCCATGGTCTTAGCGCATTTTCACTCCACAGTTACTCTCCAGGTG
AGATCAATAGGGTGGCTTCATGCCTCAGGAAACTTGGGGTACCGCCCTTG
CGAGTCTGGAGACATCGGGCCAGAAGTGTCCGCGCTAAGCTACTGTCCCA
GGGGGGGAGGGCTGCCATTTGTGGCAAGTACCTCTTCAACTGGGCRGTAA
GGACCAAGCTCAAACTCACTCCAATCCCGGCAGCGTCCCAGTTGGACTTG
TCCGACTGGTTCGTTGCCGGCTACAGCGGGGGAGACATATATCACAGCCT
GTCTCGTGCCCGACCCCGCTGGTTCCTGTGGTGCCTACTCCTGCTTTCTG
CGGGGGTAGGCATCTACTTGCTCCCCAACCGATGA
>Pt 13 NS5B
TCGATGTCCTACACATGGACAGGCGCTTTAATCACACCATGCGCTGCGGA
GGAAAGCAAGCTGCCCATCAACGCGCTGAGCAACTCCCTGCTGCGYCAC
CACAATATGGTGTATGCCACAACATCCCGCAGCGCAAGCCARCGGCAGAA
GAARGTCACTTTTGACAGACTGCAAGTCCTGGACGAYCATTACCGGGACG
TRCTCAAGGAGGTGAAGGCGAAGGCGTCCACAGTTAAGGCYAAACTTCTA
TCCGTAGAAGAGGCCTGCAAACTSACGCCCCCACACTCAGCCAAATCCAA
RTTTGGCTATGGGGCRAAGGACGTCCGGAACCTATCCAGCAAGGCCGTY
AACCACATCCACTCCGTGTGGAAGGACTTGCTGGAGGACACTGAAACACC
AATTGACACTACCATCATGGCAAAAAATGAGGTTTTCTGCGTTCAACCGG
AAAAGGGAGGCCGCAAGCCAGCTCGCCTTATCGTGTTCCCAGACCTGGGG
GTTCGTGTGTGCGAGAAAATGGCCCTCTACGACGTGGTYTCYACCCTTCC
TCAGGCCGTGATGGGCCCCTCATACGGGTTCCAGTACTCTCCTGGACAG
CGGGTCGAGTTCCTGGTGAATGCCTGGAAATCAAAGAAATGCCCTATGGG
CTTCGCATATGACACCCGCTGTTTTGACTCAACGGTCACTGAGAGTGATA
TCCGTACTGAGGAGTCTATTTACCAATGTTGTGACCTGGCCCCCGAAGCT
AGACAAGTCATAAGGTCGCTCACAGAGCGGCTTTAYATYGGGGGCCCCCT
GACYAATTCAAAAGGGCAGAACTGCGGTTATCGCCGGTGCCGYGCGAGC
GGCGTGCTGACGACTAGCTGCGGTAATACCCTCACATGTTACTTGAAGGC
CTCTGCGGCCTGTCGAGCTGCAAAGCTCCGGGACTGCACGATGCTCGTG
TGCGGAGACGACCTCGTCGTTATCTGTGAAAGCGCGGGGACCCAGGAGG
ACGCGGCTAGCCTACGAGTCTTCACGGAGGCTATGACTAGGTACTCAGCC
CCCCCCGGGGACCCGCCCCAACCAGAGTACGACTTGGAGTTGATAACAT
CATGCTCCTCCAACGTGTCGGTCGCGCACGACGCATMTGGCAAGAGGGT
GTACTACCTCACCCGTGACCCCACCACCCCCCTCGCGCGGGCTGCGTGG
GAGACAGCTAGACACACTCCAATTAACTCCTGGCTAGGCAACATCATCAT
GTATGCGCCCACYYTATGGGCAAGGATGATTCTGATGACTCACTTCTTCT
CCATCCTTCTRGCYCAGGAACAACTTGAAAAAGCCCTAGATTGCCARATC
TAYGGGGCCTGTTACTCCATTGAACCACTTGACCTACCTCAGATCATTCA
GCGACTCCATGGTCTYAGCGCATTTTCACTCCATAGTTACTCTCCAGGTG
AGATCAATAGGGTGGCTTCAAGCCTCAGGAAACTTGGGGTGCCRCCCTTG
CGAGTCTGGAGACATCGGGCCAGGAGYGTCCGCGCTAAGCTACTGTCCCA
RGGAGGGAGGGCYGCCACGTGTGGTAAGTACCTCTTCAACTGGGCAGTAA
GGACCAAGCTYAAACTCACTCCAATCCCGGCTGCGTCCCAGCTGGACTTG
TCCAGCTGGTTCGTYGCTGGTTACAGCGGGGGAGACATATATCACAGCCT
GTCTCGTGCCCGRCCCCGCTGGTTCATGTGGTGCCTACTCCTACTCTCTG
TAGGGGTAGGCATCTAYCTGCTCCCCAAYCGATGA
>Pt 14 NS5B
TCGATGTCCTACACATGGACAGGCGCCCTGATCACGCCATGCGCTGCGG
AGGAAAGCAAGCTGCCCATCAACCCGTTGAGCAACTCTTTGCTGCGTCAC
CATAAYATGGTATACGCTACAACATCCCGCAGCGCAAGCCTACGGCAGAA
GAAGGTCACTTTTGACAGACTGCAAGTCCTGGACGACCACTACCGGGACG
TGCTTAAGGAGATGAAGGCGAAGGCGTCCACAGTTAAGGCTAAGCTTCTA
TCTGTAGAAGAAGCCTGCAAACTGACACCCCCACACTCGGCCAGATCCAA
ATTTGGCTATGGGGCAAAGGACGTCCGGAGCCTATCCAGCAAGGCCGTC
AACCACATCAACTCCGTGTGGAAGGACTTGCTGGAAGACACTGAGACACC
AATTGACACCACCATCATGGCAAAAAATGAGGTTTTCTGCGTCCAACCAG
AGAAAGGAGGCCGCAAGCCAGCCCGCCTTATCGTGTTCCCAGACTTAGGG
GTTCGCGTGTGCGAGAAGATGGCCCTTTATGACGTGGTCTCCACCCTTCC
TCAGGCCGTGATGGGCTCCTCGTACGGATTCCAATACTCTCCTGGACAGC
GGGTCGAGTTCCTGGTGAATGCCTGGAAATCAAAGAAATGCCCTATGGGC
TTCTCATATGACACCCGCTGTTTTGACTCAACAGTCACCGAGAATGATAT
CCGTGTTGAGGAGTCAATTTACCAATGCTGTGACTTGGCCCCCGAAGCCA
AACAGGCCATAAGGTCGCTCACAGAGCGGCTTTAYATCGGGGGTCCCCTG
ACTAATTCAAAAGGGCAGAACTGCGGTTATCGCCGGTGCCGCGCGAGCG
GCGTGCTGACGACCAGCTGCGGTAATACCCTCACCTGTTACTTGAAGGCC
ACCGCGGCCTGTCGAGCTGCAAAGCTCCAGGACTGCACGATGCTCGTGT
GCGGGGACGACCTTGTCGTTATCTGTGAAAGCGCGGGAACCCAAGAGGA
CGCGGCGAACCTACGAGTCTTCACGGAGGCTATGACTAGGTATTCTGCCC
CCCCCGGGGACCCGCCCCAACCAGAATACGACTTGGARTTGATAACATCA
TGCTCCTCCAACGTGTCGGTCGCGCACGATGCATCTGGCAAGCGGGTGT
AYTACCTCACCCGCGACCCCACCACCCCCCTYGCACGGGCTGCGTGGGA
RACAGCTAGACACACTCCAGTTAACTCCTGGCTAGGCAACATTATCATGT
ATGCGCCCACCTTATGGGCAAGGATGATCCTGATGACTCACTTCTTCTCC
ATCCTTCTAGCTCAGGAACAACTTGAAAAAGCCCTGGATTGYCAAATCTA
CGGGGCCTGTTACTCCATTGAGCCACTTGACCTACCTCAGATCATTCAGC
GACTCCATGGCCTTAGCGCATTTTCACTCCACAGTTACTCTCCAGGTGAG
ATCAATAGGGTGGCTTCATGCCTCAGGAAACTTGGGGTACCACCCTTGCG
AGTCTGGAGACATCGGGCCAGAAGTGTCCGCGCTAAGCTACTGTCCCAGG
GAGGGAGGGCCGCCACTTGTGGCAGGTACCTCTTCAATTGGGCAGTAAGG
ACCAAGCTTAAACTCACTCCAATCCCGGCTGCGTCCCAGTTGGACTTGTC
CGGCTGGTTCGTTGCTGGGTACAGCGGGGGAGACATATATCACAGCCTGT
CTCGTGCCCGACCCCGCTGGTTCCTGTGGTGCCTACTCCTACTTTCTGTA
GGGGTAGGCATCTACCTGCTCCCCAACCGATGA
>Pt 15 NS5B
TCGATGTCCTAYACATGGACAGGCGCCCTGATCACGCCATGCGCCGCGG
ARGAAAGCAAGCTGCCCATCAATGCGTTGAGCAACTCTTTGCTGCGTCAC
CATAAYATGGTCTACGCCACAACATCCCGCAGCGCAAGCCAGCGGCAGA
AGAAGGTCACCTTTGACAGACTGCAGGTCCTGGACGACCACTACCGGGA
CGTGCTTAAGGAGATGAAGGCGAAGGCGTCCACAGTTAAGGCTAGACTTC
TATCYGTAGAAGAAGCCTGCAAGCTGACGCCCCCACACTCAGCCAGATCC
AAATTTGGCTATGGGGCGAAGGACGTCCGGAACCTATCTAGCAAGGCCGT
TAACCACATCCGCTCCGTGTGGAAGGACTTGCTGGAAGACACTGAAACAC
CAATCGACGCTACCATCATGGCAAAAAATGAGGTTTTCTGCGTCCAACCA
GAGAAAGGAGGTCGCAAGCCRGCTCGCCTTATCGTGTTCCCAGATTTGG
GAGTCCGTGTGTGCGAGAAAATGGCCCTTTACGACGTGGTCTCCACCCTT
CCTCAGGCCGTGATGGGCCCCTCATACGGATTCCAATACTCTCCTGGACA
GCGGGTCGAGTTCCTGGTGAATGCCTGGAAATCAAAGAAAAACCCTATGG
GCTTCTCATATGACACCCGCTGYTTTGACTCTACGGTCACYGAGAGYGAC
ATCCGTACTGAGGAGTCAATTTACCAATGTTGTGACTTGGCCCCCGAAGC
CAGACAGGTTATAAGGTCGCTCACAGAGCGGCTTTATATCGGGGGTCCTY
TGACTAATTCAAAAGGGCAGAACTGCGGCTATCGCCGGTGTCGCGCAAG
CGGCGTGCTGACGACCAGCTGCGGCAATACCCTCACATGTTACCTGAAG
GCCACTGCAGCCTGTCGAGCTGCGAAGCTCCAGGACTGCACAATGCTTG
TGTGTGGGGACGACCTTGTCGTYATCTGTGAGAGCGCGGGGACCCAAGA
GGACGCAGCGAGCCTACGAGTCTTCACGGAGGCTATGACTAGGTACTCT
GCTCCCCCCGGGGACCCGCCCCGGCCGGAATACGACTTGGARTTAATAA
CATCATGCTCCTCCAACGTGTCGGTCGCGCACGACGCACAYGGCAAAAG
GGTGTACTACCTCACCCGTGACCCCACCACCCCCCTTGCGCGGGCYGCA
TGGGAGACAGCTAGACACACTCCAGTCAACTCCTGGCTAGGCAACATCAT
CATGTATGCGCCCACCTTGTGGGCAAGGATGATYCTGATGACYCATTTCT
TCTCCATCCTTCTAGCCCAGGAGCAACTTGAAAAAGCCCTAGATTGTCAG
ATCTACGGGGCCTGTTACTCCATTGAGCCACTTGACCTACCTCAGATCAT
TCAGCGACTCCATGGTCTTAGCGCATTTTCACTCCACAGTTACTCTCCAG
GTGAGATCAATAGGGTGGCTTCATGCCTCAGGAAACTTGGGGTACCACCC
CTGCGAGTCTGGAGACATCGGGCCAGAAGTGTCCGCGCTAAGCTGCTGTC
CCGGGGGGGGAGGGCTGCCACTTGTGGCAAGTACCTCTTCAACTGGGCRG
TAAGGACCAAGCTCAAACTCACTCCAATCCCGGCTGCGTTCAAGCTGGAC
TTGTCCGGCTGGTTCGTTGCTGGTTACAGCGGGGGAGACATATATCACAG
CCTGTCTCGTGCCCGACCCCGCTGGTTYRTGTGGTGCCTACTCCTACTTT
CTGTAGGGGTAGGCATCTACCTGCTCCCCAACCGATGA
>Pt 16 NS5B
TCGATGTCCTACACATGGACAGGCGCCTTGATCACACCGTGCGCTGCGG
ARGAGAGCAAGCTGCCCATCAAYGCGCTGAGCAACTCTTTGYTGCGYCAC
CATAACATGRTCTATGCCACAACATCCCGCAGCGCYAGCCAAMGGCAGAR
GAAGGTCACTTTTGAYAGACTGCARGTCCTGGACGACCACTACCGGGACG
TGCTYAAGGAGATGAAGGCGAAGGCGTCCACAGTCAAGGCTAAACTTCTA
TCCGTAGARGAAGCCTGYAAGCTGACRCCCCCACACTCGGCCAGATCYAA
ATTTGGCTATGGGGCAAAGGACGTCCGGAACCTATCCAGCAAGGCCGTTA
ACCACATCCACTCCGTGTGGAAGGACTTGCTGGAAGACACTGACACACCA
ATTGACACCACCATCATGGCAAAAAATGAGGTTTTCTGYATCCAACCAGA
GAAAGGAGGCCGCAAGCCAGCTCGCCTTATCGTRTACCCAGACCTGGGGG
TCCGRGTGTGCGAGAAGATGGCTCTTTAYGATGTGGTCTCCACYCTTCCT
CAGGCCGTGATGGGCCCCTCRTACGGATTTCAGTACTCTCCTGGACAGC
GGGTTGAGTTCCTGGTGAAWGCCTGGAARTCAAAGAAATGCCCTATGGG
CTTCGCRTATGACACCCGCTGCTTYGACTCRACGGTCACTGAGAATGACA
TYCGTGTTGAGGAGTCAATTTACCAATGTTGTGACTTGGCYCCCGAAGCC
AGACAGGYCATAAGGTCGCTCACAGAGCGGCTTTAYATCGGGGGTCCYCT
AACCAATTCAAAAGGGCAAAACTGCGGTTATCGCCGGTGTCGCGCRAGC
GGCGTGCTGACGACTAGCTGCGGCAAYACCCTTACATGTTACTTGAARGC
CTCTGCRGCCTGTCGAGCTGCGAAGCTCCAGGACTGCACGATGCTCGTG
TGCGGAGACGACCTCGTCGTTATCTGTGAGAGCGCGGGGACCCACGAGG
ATGCGGCGAGCCTACGAGTCTTYACGGAGGCTATGACTAGGTACTCCGC
CCCCCCYGGGGACCCGCCTCAGCCAGAATACGACTTAGAGCTGATAACAT
CATGCTCTTCCAAYGTGTCRGTCGCGCACGATGCATCYGGCAAAAGGGTR
TACTACCTCACCCGTGACCCCACCACCCCCCTTGCRCGGGCTGCGTGGG
ARACAGCTAGACACACTCCAGTYAACTCCTGGCTAGGCAACATCATCATG
TAYGCGCCCACCYTATGGGCAAGGATGATCCTGATGACTCATTTCTTCTC
CATCCTTCTAGCTCAGGAGCAACTTGAAAAAGCCCTAGATTGTCAGATCT
AYGGGGCCTGTTACTCCATTGAACCACTTGACCTACCTCAAATCATTCAR
CGACTCCATGGTATTAGCGCGTTTTCACTCCAYAGTTACTCTCCAGGWGA
GATCAATAGGGTGGCTTCATGCCTCAGGAAACTTGGGGTACCRCCCTTGC
GAGTCTGGAGACATCGGGCCAGGAGTGTCCGCGCTAAGYTACTGTCCCAG
GGGGGGAGGGCTGCCACTTGTGGCAARTACCTCTTCAACTGGGCAGTAAR
AACCAAGCTTAATCTCACTCCAATTCCGGCTGCGTCCAAGCTGGATTTAT
CCRGCTGGTTCGTTGCCGGYTACAGCGGGGGAGACATATATCACAGCGTG
TCTCMTGCCCGACCCCGCTGGTTCATGTGGTGCCTRCTCCTACTKTCTGT
AGGRGTAGGCATCTACCTGCTYCCCAACCGATGA

D. Alignment of Sequences with Reference Sequence

The alignment shows the nucleotide sequence of the NS5B polymerase domain of an HCV-1b isolate from an untreated patient. The sequence was aligned against a reference sequence. Homologies between the two sequences are plotted as dots.

| | |
|---|---|
| | 10 20 30 40 |
| | ....\|....\|....\|....\|....\|....\|....\|....\| |
| con1b reference | tcgatgtcctacacatggacaggcgccctgatcacgccat |
| Pt 12 NS5B | ..............G.....G.................G. |
| | 50 60 70 80 |
| | ....\|....\|....\|....\|....\|....\|....\|....\| |
| con1b reference | gcgctgcggaggaaaccaagctgcccatcaatgcactgag |
| Pt 12 NS5B | ....C..........G.........T.........T.... |
| | 90 100 110 120 |
| | ....\|....\|....\|....\|....\|....\|....\|....\| |
| con1b reference | caactctttgctccgtcaccacaacttggtctatgctaca |
| Pt 12 NS5B | ......AC....G...........TA....T......... |
| | 130 140 150 160 |
| | ....\|....\|....\|....\|....\|....\|....\|....\| |
| con1b reference | acatctcgcagcgcaagcctgcggcagaagaaggtcacct |
| Pt 12 NS5B | .....C.............A..................T. |
| | 170 180 190 200 |
| | ....\|....\|....\|....\|....\|....\|....\|....\| |
| con1b reference | ttgacagactgcaggtcctggacgaccactaccgggacgt |
| Pt 12 NS5B | .............A.......................... |
| | 210 220 230 240 |
| | ....\|....\|....\|....\|....\|....\|....\|....\| |
| con1b reference | gctcaaggagatgaaggcgaaggcgtccacagttaaggct |
| Pt 12 NS5B | ........................................ |
| | 250 260 270 280 |
| | ....\|....\|....\|....\|....\|....\|....\|....\| |
| con1b reference | aaacttctatccgtggaggaagcctgtaagctgacgcccc |
| Pt 12 NS5B | ..G........T..A..............A.......... |
| | 290 300 310 320 |
| | ....\|....\|....\|....\|....\|....\|....\|....\| |
| con1b reference | cacattcggccagatctaaatttggctatggggcaaagga |
| Pt 12 NS5B | ................C...........Y........... |
| | 330 340 350 360 |
| | ....\|....\|....\|....\|....\|....\|....\|....\| |
| con1b reference | cgtccggaacctatccagcaaggccgttaaccacatccgc |
| Pt 12 NS5B | ........................................ |
| | 370 380 390 400 |
| | ....\|....\|....\|....\|....\|....\|....\|....\| |
| con1b reference | tccgtgtggaaggacttgctggaagacactgagacaccaa |
| Pt 12 NS5B | ........................................ |
| | 410 420 430 440 |
| | ....\|....\|....\|....\|....\|....\|....\|....\| |
| con1b reference | ttgacaccaccatcatggcaaaaaatgaggttttctgcgt |
| Pt 12 NS5B | .........................C.....Y........ |
| | 450 460 470 480 |
| | ....\|....\|....\|....\|....\|....\|....\|....\| |
| con1b reference | ccaaccagagaaggggggccgcaagccagctcgccttatc |
| Pt 12 NS5B | ............A..A........................ |
| | 490 500 510 520 |
| | ....\|....\|....\|....\|....\|....\|....\|....\| |
| con1b reference | gtattcccagatttgggggttcgtgtgtgcgagaaaatgg |
| Pt 12 NS5B | ..G........C.....A...................... |
| | 530 540 550 560 |
| | ....\|....\|....\|....\|....\|....\|....\|....\| |
| con1b reference | ccctttacgatgtggtctccaccctccctcaggccgtgat |
| Pt 12 NS5B | ..........C...........T..T.....A........ |
| | 570 580 590 600 |
| | ....\|....\|....\|....\|....\|....\|....\|....\| |
| con1b reference | gggctcttcatacggattccaatactctcctggacagcgg |
| Pt 12 NS5B | ......C.....T........G.................. |
| | 610 620 630 640 |
| | ....\|....\|....\|....\|....\|....\|....\|....\| |
| con1b reference | gtcgagttcctggtgaatgcctggaaagcgaagaaatgcc |
| Pt 12 NS5B | ..T..A....................GT.......GAA.. |
| | 650 660 670 680 |
| | ....\|....\|....\|....\|....\|....\|....\|....\| |
| con1b reference | ctatgggcttcgcatatgacacccgctgttttgactcaac |
| Pt 12 NS5B | ........................................ |
| | 690 700 710 720 |
| | ....\|....\|....\|....\|....\|....\|....\|....\| |
| con1b reference | ggtcactgagaatgacatccgtgttgaggagtcaatctac |
| Pt 12 NS5B | A..........G.........C.................. |
| | 730 740 750 760 |
| | ....\|....\|....\|....\|....\|....\|....\|....\| |
| con1b reference | caatgttgtgacttggcccccgaagccagacaggccataa |
| Pt 12 NS5B | ............................A........... |
| | 770 780 790 800 |
| | ....\|....\|....\|....\|....\|....\|....\|....\| |
| con1b reference | ggtcgctcacagagcggctttacatcgggggccccctgac |
| Pt 12 NS5B | A..............................T........ |
| | 810 820 830 840 |
| | ....\|....\|....\|....\|....\|....\|....\|....\| |
| con1b reference | taattctaaagggcagaactgcggctatcgccggtgccgc |
| Pt 12 NS5B | ......A................................. |
| | 850 860 870 880 |
| | ....\|....\|....\|....\|....\|....\|....\|....\| |
| con1b reference | gcgagcggtgtactgacgaccagctgcggtaataccctca |
| Pt 12 NS5B | ..C.....C.................T............. |
| | 890 900 910 920 |
| | ....\|....\|....\|....\|....\|....\|....\|....\| |
| con1b reference | catgttacttgaaggccgctgcggcctgtcgagctgcgaa |
| Pt 12 NS5B | .............A...T...................A.. |
| | 930 940 950 960 |
| | ....\|....\|....\|....\|....\|....\|....\|....\| |
| con1b reference | gctccaggactgcacgatgctcgtatgcggagacgacctt |
| Pt 12 NS5B | ........................G............... |
| | 970 980 990 |
| 1000 | |
| | ....\|....\|....\|....\|....\|....\|....\|....\| |
| con1b reference | gtcgttatctgtgaaagcgcggggacccaagaggacgagg |
| Pt 12 NS5B | ..............G........A.....G.......C.. |
| | 1010 1020 1030 |
| 1040 | |
| | ....\|....\|....\|....\|....\|....\|....\|....\| |
| con1b reference | cgagcctacgggccttcacggaggctatgactagatactc |
| Pt 12 NS5B | ..........A.T.....................G..... |
| | 1050 1060 1070 |
| 1080 | |
| | ....\|....\|....\|....\|....\|....\|....\|....\| |
| con1b reference | tgccccccctggggacccgcccaaaccagaatacgacttg |
| Pt 12 NS5B | C........C............C.G.....G......... |
| | 1090 1100 1110 |
| 1120 | |
| | ....\|....\|....\|....\|....\|....\|....\|....\| |
| con1b reference | gagttgataacatcatgctcctccaatgtgtcagtcgcgc |
| Pt 12 NS5B | ..........................C.....G....... |
| | 1130 1140 1150 |
| 1160 | |
| | ....\|....\|....\|....\|....\|....\|....\|....\| |
| con1b reference | acgatgcatctggcaaaagggtgtactatctcacccgtga |
| Pt 12 NS5B | ..........C......C.......T..C........... |
| | 1170 1180 1190 |
| 1200 | |
| | ....\|....\|....\|....\|....\|....\|....\|....\| |
| con1b reference | ccccaccaccccccttgcgcgggctgcgtgggagacagct |
| Pt 12 NS5B | ...............C...A.............A...... |
| | 1210 1220 1230 |
| 1240 | |
| | ....\|....\|....\|....\|....\|....\|....\|....\| |
| con1b reference | agacacactccagtcaattcctggctaggcaacatcatca |
| Pt 12 NS5B | ..............T.....T.................T. |
| | 1250 1260 1270 |
| 1280 | |
| | ....\|....\|....\|....\|....\|....\|....\|....\| |
| con1b reference | tgtatgcgcccaccttgtgggcaaggatgatcctgatgac |
| Pt 12 NS5B | ..............C................TT....... |
| | 1290 1300 1310 |
| 1320 | |
| | ....\|....\|....\|....\|....\|....\|....\|....\| |
| con1b reference | tcatttcttctccatccttctagctcaggaacaacttgaa |
| Pt 12 NS5B | ...C.......................A............ |
| | 1330 1340 1350 |
| 1360 | |
| | ....\|....\|....\|....\|....\|....\|....\|....\| |
| con1b reference | aaagccctagattgtcagatctacggggcctgttactcca |
| Pt 12 NS5B | ........G.......................C....... |
| | 1370 1380 1390 |
| 1400 | |
| | ....\|....\|....\|....\|....\|....\|....\|....\| |
| con1b reference | ttgagccacttgacctacctcagatcattcaacgactcca |
| Pt 12 NS5B | ...............................R........ |
| | 1410 1420 1430 |
| 1440 | |
| | ....\|....\|....\|....\|....\|....\|....\|....\| |
| con1b reference | tggccttagcgcattttcactccatagttactctccaggt |
| Pt 12 NS5B | ...T....................C............... |
| | 1450 1460 1470 |
| 1480 | |
| | ....\|....\|....\|....\|....\|....\|....\|....\| |
| con1b reference | gagatcaatagggtggcttcatgcctcaggaaacttgggg |
| Pt 12 NS5B | ........................................ |
| | 1490 1500 1510 |
| 1520 | |
| | ....\|....\|....\|....\|....\|....\|....\|....\| |
| con1b reference | taccgcccttgcgagtctggagacatcgggccagaagtgt |
| Pt 12 NS5B | ........................................ |
| | 1530 1540 1550 |
| 1560 | |
| | ....\|....\|....\|....\|....\|....\|....\|....\| |
| con1b reference | ccgcgctaggctactgtcccagggggggagggctgccact |
| Pt 12 NS5B | ........A.............................T. |
| | 1570 1580 1590 |
| 1600 | |
| | ....\|....\|....\|....\|....\|....\|....\|....\| |
| con1b reference | tgtggcaagtacctcttcaactgggcagtaaggaccaagc |
| Pt 12 NS5B | ..........................R............. |
| | 1610 1620 1630 |
| 1640 | |
| | ....\|....\|....\|....\|....\|....\|....\|....\| |
| con1b reference | tcaaactcactccaatcccggctgcgtcccagttggattt |
| Pt 12 NS5B | ......................A..............C.. |
| | 1650 1660 1670 |
| 1680 | |
| | ....\|....\|....\|....\|....\|....\|....\|....\| |
| con1b reference | atccagctggttcgttgctggttacagcgggggagacata |
| Pt 12 NS5B | G...GA............C..C.................. |
| | 1690 1700 1710 |
| 1720 | |
| | ....\|....\|....\|....\|....\|....\|....\|....\| |
| con1b reference | tatcacagcctgtctcgtgcccgaccccgctggttcatgt |
| Pt 12 NS5B | ....................................C... |
| | 1730 1740 1750 |
| 1760 | |
| | ....\|....\|....\|....\|....\|....\|....\|....\| |
| con1b reference | ggtgcctactcctactttctgtaggggtaggcatctatct |
| Pt 12 NS5B | .............G.......CG..............CT. |
| | 1770 |
| | ....\|....\|....\|. |
| con1b reference | actccccaaccgatga |
| Pt 12 NS5B | G............... |

### Example 3

### NS3 Phenotyping Assay

### Construction of Delta [NS3] Shuttle Vector

The plasmid 11 pFK I341 PI luc NS3-3′_ET is based on the construct described in Krieger et al. 2001 and was kindly provided by Prof. Bartenschlager (Heidelberg, Germany). In order to generate a shuttle vector for NS3 phenotyping, it was modified by site-directed mutagenesis to introduce two new SacII restriction sites at position 3338 and 3899. In a next step, the modified plasmid was digested with SacII and subsequently religated to give the delta[NS3] shuttle vector pFK I341 PI luc ΔNS3 7-192_ET (SEQ ID No 10).

For InFusion cloning, the delta[NS3] backbone pFK I341 PI luc ΔNS3 7-192_ET (SEQ ID NO: 10) was linearized by SacII digestion.

### Example 4

### Cloning of the NS3 PCR Amplicons from Infected Patients into the Delta[NS3] Shuttle Vector

A. NS3 Amplicon Generation from Isolates of HCV-Infected Patients

For the PCR, 1 μl from the One-Step RT-PCR product of the NS3 genotyping assay was mixed with 0.2 μM primer 1b_InFu_NS3_F (SEQ ID NO: 11), 0.2 μM primer 1b_InFu_NS3_R (SEQ ID NO: 12) and 2× Herculase™ Hotstart master mix (Stratagene) to give a total volume of 50 μl. Initial denaturation was 95° C. for 2 min and thermal cycling consisted of 10 cycles followed by another 20 cycles consisting of denaturation at 95° C. for 30 s, annealing at 60° C. for 30 s and elongation at 72° C. for 1 min (plus 10 s per cycle). Final extension took place at 72° C. for 10 min. The amplicons were purified using the QIAQuick gel purification kit (Qiagen).

### B. Delta [NS3] Shuttle Vector Preparation

The NS3 subgenomic shuttle backbone was digested with an excess of the restriction endonuclease SacII (NEB) and 1× restriction enzyme buffer 4 (NEB) at 37° C. overnight. In a next step, calf intestine phosphatase was added and the mixture incubated for 40 min at 37° C. in order to dephosphorylate the linearized shuttle backbone. The dephosphorylated vector was purified via agarose gel electrophoresis (crystal violet) followed by gel extraction using the kit from QIAGEN. The linearized vector was stored at −20° C. until further use.

C. Cloning of the NS3 Derived from Patient Isolates into the Linearized Delta [NS3] Shuttle Vector

The PCR products and the linearized vector were thawed and the PCR product was stored on ice until cloning. Immediately before the In-Fusion™ cloning, the linearized vector was denatured for 5 min at 60° C. and subsequently put on ice. For the cloning reaction, 1 μl of the PCR product and 1 μl of the vector preparation were added to 8 μl Dnase/Rnase-free water. The complete mix (10 μl) was added into one tube containing the Dry-Down In-Fusion™ reaction mix (Clontech) and carefully pipetted up and down. The pipetting steps were performed on ice. The PCR tubes containing the In-Fusion™ cloning mix were subsequently transferred to a thermocycler and incubated for 30 min at 42° C. After incubation the tubes were immediately transferred to ice.

### D. Transformation of Recombinant Replicon DNA

The transformation of Escherichia coli cell was performed immediately after the In-Fusion™ cloning step. The XL10-Gold® Ultracompetent Cells (Stratagene) were used for the transformation. 50 μl of the cells were transformed with 5 μl of the In-Fusion™ cloning mix according to the protocol from Stratagene. The complete transformation mix was plated onto ampicillin-containing LB Petri dishes and incubated overnight at 37° C. Colonies were pooled by applying 1 ml of ampicillin-containing LB medium onto the Petri dishes and removing the colonies by scraping. The bacterial suspension was transferred into a 15 ml-Falcon tube. The Petri dishes were washed for a second time with 1 ml of the ampicillin-containing LB medium and the solution was again transferred into the Falcon tube. 2 ml of ampicillin-containing LB medium were added and cells were grown at 37° C. until they reached the logarithmic phase (approximately 4-5 hours). 1.5 ml of the cell culture was used for inoculation of 200 ml ampicillin-containing LB medium. Cells were grown overnight at 37° C. for the DNA preparation. The DNA was prepared using the Maxiprep DNA purification kit from QIAGEN.

### Example 5

### Replicon NS3 Phenotypic Assay

### A. Recombinant Replicon Plasmid DNA Linearization

The replicon plasmid DNA (10 μg per sample) was linearized using 1.5 μl of AseI (NEB) and 3 μl ScaI (NEB) together with 4 μl NEB buffer 3 to give a total volume of 40 μl. The reaction mix was incubated for 4 hours at 37° C. The linearized vector was separated from the resulting fragments via agarose gel electrophoresis and purified using the gel extraction kit from QIAGEN. DNA concentration was measured using the Nanodrop® spectrophotometer (ratio OD260 nm/OD280 nm). The purified DNA was stored at −20° C. until further use.

### B. Preparation of In Vitro Transcribed Replicon RNA

The in vitro transcription was performed using the MEGAscript High Yield Transcription kit (Ambion) according to protocol HCV_SP_038.vs2 in the Laboratory Operation Unit at Tibotec. Briefly, 1 μg of the linearized and purified replicon DNA was used per reaction for in vitro transcription and were added to a mix containing 44 μl nuclease-free water, 4 μl ATP solution, 4 μl CTP solution, 4 μl GTP solution, 4 μl UTP solution and 10× reaction buffer. Four μl of the enzyme mix were subsequently added. The pipetting was performed at room temperature. The reaction mix was incubated for 4 hours at 37° C. Two pi of TURBO DNase (Ambion) were subsequently added and the mixture was incubated for 15 min at 37° C. in order two destroy the DNA template. The RNA was purified using the MEGAclear™ kit (Ambion). RNA was quantified using the Nanodrop® spectrophotometer (ratio OD260 nm/OD280 nm). The purified RNA was stored in 10 μg aliquots at −80° C. until further use.

### C. Hepatoma Cell Line

Cured hepatoma cell line Huh7 were cultured at 37° C. in a humidified atmosphere with 5% CO₂ in Dulbecco's Modified Eagle medium (DMEM, Biowhittaker, Cat no. BE12-917F) supplemented with L-Glutamine and 10% FCS.

### D. Determination of Transient Replicon Replication

4×10⁶ cells were transfected with 10 μg of in vitro transcribed replicon RNA via electroporation. For EC₅₀ determination 4,000 cells/well were seeded in a volume of 30 μl medium in white 384-well compound plates. Compound plates contained 10 μl/well of the respective compound dilution in medium (containing 2% DMSO), leading to a total volume of 40 μl per well with a final concentration of 0.5% DMSO. Compound dilutions were prepared in quadruplates. Cell culture plates were incubated for 48 h at 37° C. and 5% CO₂. Experiments were performed in triplicates. The firefly luciferase chemiluminescence read-out was performed using the Steady-Lite reagent (PerkinElmer). The EC₅₀ values were assessed as the inhibitor concentration at which a 50% reduction in the level of firefly luciferase reporter was observed as compared to the level of firefly luciferase signal without the addition of compounds. Results of studies testing the inhibitory effect of an example protease inhibitor, SCH 503034, on replication of WT replicon and replicons with patient-derived NS3 sequences are shown in Table 2.

Table 2 shows that the NS3-restored shuttle vector is replicating. GND serves as a non-replicating replicon control.

Table 3 shows EC₅₀ values of an HCV protease inhibitor tested in the NS3 replicon shuttle system with 5 patient isolates.

### Results:

TABLE 2
Replication level of replicons (96-well format*)

| | | | Replication |
|---|---|---|---|
| Plasmid | Vector backbone | RLU level¹ | level² |
| rep Pl-luc/ET (WT) | rep Pl-luc/ET (WT) | 1637 ± 348 | |
| rep Pl-luc/ET NS3 | Rep Pl-luc/ET delta | 1047 ± 151 | WT level |
| 7-192 InFu restored | [NS3 7-192] SaclI | | |
| GND | | 17 ± 2 | No replication |

15000 cells were seeded per well in 96-well plates.
¹RLU represents level of firefly luciferase signal observed after 48 hours post-transfection.
²Replication level is compared to wild type (WT) vector.

TABLE 3
EC₅₀ values (384-well format)

| NS3 sequence | SCH 503034 EC₅₀ [μM]* |
|---|---|
| rep Pl-luc/ET (WT) | 0.140 ± 0.069 |
| Clinical isolate Pt 1 | 0.341 ± 0.130 |
| Clinical isolate Pt 2 | 0.090 ± 0.046 |
| Clinical isolate Pt 3 | 0.124 ± 0.023 |
| Clinical isolate Pt 4 | 0.126 ± 0.018 |
| Clinical isolate Pt 5 | 0.120 ± 0.068 |

*Inhibition by SCH 503034 of transient HCV replicon RNA replication containing the NS3 from genotype 1b clinical isolates inserted into the shuttle vector pFK Pl-luc delta[NS3 7-192]_ET; mean EC₅₀ value from at least n = 3 experiments.

### Example 6

### NS5B Phenotyping Assay

### Construction of Delta [NS5B] Backbone

The plasmid 11pFK I341 PI luc NS3-3′_ET is based on the construct described in Krieger et al. 2001 and was kindly provided by Prof. Bartenschlager (Heidelberg, Germany). In order to generate a shuttle vector, it was modified by site-directed mutagenesis to introduce two AflII restriction sites at position 7481 and 9287. First, an AflII restriction site was introduced by site directed mutagenesis into the 3′ NCR directly after the stop codon of NS5B at Medigenomix (Munich, Germany) resulting in plasmid pFKi341Luc_NS3-3′-ET-AflII (Sequence ID NO:17). Next, a second AflII restriction site 8 aa upstream of the NS5A/NS5B cleavage site was introduced using the Quick Change Site-Directed Mutagenesis Kit (Stratagene, La Jolla, Calif., USA) according to the manufacturers recommendations with SDM primer pair AflII-5A-fwd (5′-accgtaagcgaggagcttaaggctagtgaggacgtc-3′) (Sequence ID NO:18) and AflII-5A-rev (5′-gacgtcctcactagccttaagctcctcgcttacggt-3′) (Sequence ID NO:19) resulting in plasmid pFKi341Luc_NS3-3′-ET-2×AflII (Sequence ID NO:20). In a next step, the modified plasmid was digested with AflII and subsequently re-ligated resulting in the delta[NS5B] backbone pFK_I341_PI_NS3-3_ET_dNS5a/b_5a440-5b591-ScaI (Sequence ID NO:21).

In parallel, a NS5B phenotyping construct with an XbaI restriction site at the 3′ end was generated using plasmid pFKi341Luc_NS3-3′-ET-2×AflII (Sequence ID NO:20) as template. First, an XbaI site in the gene of the firefly luciferase was mutated by a site directed mutagenesis approach, resulting in a silent mutation, using primer pair XbaI-mut-fwd (5′-ggcgccattctatccactagaggatggaacc-3′) (Sequence ID NO:22) and XbaI-mut-rev (5′-ggttccatcctctagtggatagaatggcgcc-3′) (Sequence ID NO:23). In a second SDM reaction, an XbaI restriction site was introduced at the 3′ end of the HCV 3′ NCR instead of the ScaI site using primer pair XbaI-add-fwd (5′-gagtgctgatactggcctctctgcagatcaagtctagaaagtccctttagtgagggttaattc-3′) (Sequence ID NO:24) and XbaI-add-rev (5′-gaattaaccctcactaaagggactttctagacttgatctgcagagaggccagtatcagcactc-3′) (Sequence ID NO:25) resulting in plasmid pFKi341Luc_NS3-3′-ET-2×Afl II-XbaI (Sequence ID NO:26). In a next step, the modified plasmid was digested with AflII and subsequently re-ligated resulting in the delta[NS5B] backbone pFK_I341_PI_NS3-3_ET_dNS5a/b_5a440-5b591-XbaI (Sequence ID NO:27). Linearization with XbaI results in an authentic HCV 3′ end and offered the possibility to shuttle amplicons of clinical isolates which harbor a ScaI site in the NS5B coding sequence.

For InFusion cloning, the delta[NS5B] backbone pFK_I341_PI_NS3-3_ET_dNS5a/b_5a440-5b591-ScaI (SEQ ID NO:21) or pFK_I341_PI_NS3-3_ET_dNS5a/b_5a440-5b591-XbaI (SEQ ID NO:27) was linearized by AflII digestion.

### Example 7

### Cloning of the NS5B PCR Amplicons from HCV-Infected Patients into the Delta [NS5B] Shuttle Vector

A. NS5B Amplicon Generation from Isolates of HCV-Infected Patients

For the InFusion™ PCR, 1 μl from the One-Step RT-PCR product of the NS5B genotyping assay was mixed with 0.2 μM primer 1b_NS5B_F_AflII-Infusion (5′-AAGCGAGGAGCTTAAGGCYRGTGAGGACGT-3′) (SEQ ID NO:28), 0.2 μM primer 1b_NS5B_R_AflII-Infusion (5′-AGCTCCCCGTCTTAAGTCAYCGGT TGGGG-3′) (SEQ ID NO:29) and 2× Herculase™ Hotstart master mix (Stratagene, La Jolla, Calif., U.S.) to give a total volume of 50 μl. Initial denaturation was 95° C. for 2 min and thermal cycling consisted of 10 cycles followed by another 20 cycles consisting of denaturation at 95° C. for 30 s, annealing at 60° C. for 30 s and elongation at 72° C. for 1 min 30 s (plus 10 s per cycle). Final extension took place at 72° C. for 10 min. The amplicons were purified using the QIAQuick gel purification kit (Qiagen, Hilden, Germany). Final volume of purified amplicons was 30 μl.

### B. Delta [NS5B] Shuttle Vector Preparation

The NS5B subgenomic shuttle backbone was digested with an excess of the restriction endonuclease AflII (NEB) and 1× restriction enzyme buffer 4 (NEB) at 37° C. overnight. In a next step, calf intestine phosphatase was added and the mixture incubated for 1 h at 37° C. in order to dephosphorylate the linearized shuttle backbone. The dephosphorylated vector was purified via agarose gel electrophoresis (crystal violet) followed by gel extraction using the kit from QIAGEN. The linearized vector was stored at −20° C. until further use.

C. Cloning of the NS5B Derived from Patient Isolates into the Linearized Delta [NS5B] Shuttle Vector

The PCR products and the linearized vector were thawed and the PCR product was stored on ice until cloning. Immediately before the In-Fusion™ cloning, the linearized vector was denatured for 5 min at 60° C. and subsequently put on ice. For the cloning reaction, 2 μl of the PCR product and 1-3 μl of the vector preparation were added to 5-8 μl Dnase/Rnase-free water. The complete mix (10 μl) was added into one tube containing the Dry-Down In-Fusion™ reaction mix (Clontech) and carefully pipetted up and down. The pipetting steps were performed on ice. The PCR tubes containing the In-Fusion™ cloning mix were subsequently transferred to a thermocycler and incubated for 30 min at 42° C. After incubation the tubes were immediately transferred to ice

### D. Transformation of Recombinant Replicon DNA

The transformation of Escherichia coli cell was performed immediately after the In-Fusion™ cloning step. The XL10-Gold® Ultracompetent Cells (Stratagene) were used for the transformation. 50 μl of the cells were transformed with 5 of the In-Fusion™ cloning mix according to the protocol from Stratagene. The complete transformation mix was plated onto ampicillin-containing LB Petri dishes and incubated overnight at 37° C. Colonies were pooled by applying 1 ml of ampicillin-containing LB medium onto the Petri dishes and removing the colonies by scraping. The bacterial suspension was transferred into a 15 ml-Falcon tube. The Petri dishes were washed for a second time with 1 ml of the ampicillin-containing LB medium and the solution was again transferred into the Falcon tube. 2 ml of ampicillin-containing LB medium were added and cells were grown at 37° C. until they reached the logarithmic phase (approximately 4-5 hours). 1.5 ml of the cell culture was used for inoculation of 200 ml ampicillin-containing LB medium. Cells were grown overnight at 37° C. for the DNA preparation. The DNA was prepared using the Maxiprep DNA purification kit from QIAGEN.

### Example 8

### Replicon NS5B Phenotypic Assay

### A. Recombinant Replicon Plasmid DNA Linearization

The replicon plasmid DNA (10 μg per sample) was linearized using 3 μl of XbaI (NEB) together with 10 μl NEB buffer 4 and 1 μl of a 100× concentrated BSA stock solution (NEB) to give a total volume of 100 μl. The reaction mix was incubated for 4 hours at 37° C. The linearized vector was separated from the resulting fragments via agarose gel electrophoresis and purified using the gel extraction kit from QIAGEN. DNA concentration was measured using the Nanodrop® spectrophotometer (ratio OD260 nm/OD280 nm). The purified DNA was stored at −20° C. until further use.

### B. Preparation of In Vitro Transcribed Replicon RNA

The in vitro transcription was performed using the MEGAscript High Yield Transcription kit (Ambion) according to protocol HCV_SP_038.vs2 in the Laboratory Operation Unit at Tibotec. Briefly, 1 μg of the linearized and purified replicon DNA was used per reaction for in vitro transcription and were added to a mix containing 44 μl nuclease-free water, 4 μl ATP solution, 4 μl CTP solution, 4 μl GTP solution, 4 μl UTP solution and 10× reaction buffer. Four μl of the enzyme mix were subsequently added. The pipetting was performed at room temperature. The reaction mix was incubated for 4 hours at 37° C. Two pi of TURBO DNase (Ambion) were subsequently added and the mixture was incubated for 15 min at 37° C. in order two destroy the DNA template. The RNA was purified using the MEGAclear™ kit (Ambion). RNA was quantified using the Nanodrop® spectrophotometer (ratio OD260 nm/OD280 nm). The purified RNA was stored in 10 μg aliquots at −80° C. until further use.

### C. Hepatoma Cell Line

Cured hepatoma cell line Huh7 were cultured at 37° C. in a humidified atmosphere with 5% CO₂ in Dulbecco's Modified Eagle medium (DMEM, Biowhittaker, Cat no. BE12-917F) supplemented with L-Glutamine and 10% FCS.

### D. Determination of Transient Replicon Replication

4×10⁶ cells were transfected with 10 μg of in vitro transcribed replicon RNA via electroporation. For EC₅₀ determination 4,000 cells/well were seeded in a volume of 30 μl medium in white 384-well compound plates. Compound plates contained 10 μl/well of the respective compound dilution in medium (containing 2% DMSO), leading to a total volume of 40 μl per well with a final concentration of 0.5% DMSO. Compound dilutions were prepared in quadruplates. Cell culture plates were incubated for 48 h at 37° C. and 5% CO₂. Experiments were performed in at least duplicates. The firefly luciferase chemiluminescence read-out was performed using the Steady-Lite reagent (PerkinElmer). The EC₅₀ values were assessed as the inhibitor concentration at which a 50% reduction in the level of firefly luciferase reporter was observed as compared to the level of firefly luciferase signal without the addition of compounds. Results of studies testing the inhibitory effect of an example polymerase inhibitor, Thiophene-2-carboxylic acid, on replication of WT replicon and replicons with patient-derived NS5B sequences are shown in Table 4.

Table 4 shows EC₅₀ values of an HCV polymerase inhibitor tested in the NS5B replicon shuttle system with 5 patient isolates.

### Results:

TABLE 4
EC₅₀ values (384-well format)

| NS5B sequence | Thiophene-2-carboxylic acid EC₅₀ [μM]* |
|---|---|
| Rep Pl-luc/ET (WT) | 0.58 |
| Clinical isolate Pt_12 | 0.28 |
| Clinical isolate Pt_13 | 0.59 |
| Clinical isolate Pt_14 | 1.0** |
| Clinical isolate Pt_15 | 0.63 |
| Clinical isolate Pt_16 | 3.96 |

*Inhibition by Thiophene-2-carboxylic acid of transient HCV replicon RNA replication containing the NS5B from genotype 1b clinical isolates inserted into the shuttle vector pFK Pl-luc delta[NS5B]_ET; mean EC₅₀ value from at least n = 2 experiments .
**measured once

TABLE 5

| SEQ | | | | Amplification/ |
|---|---|---|---|---|
| ID NO | Primer name | Sequence (5′ to 3′) | Remark | Sequencing |
| 1 | NS5Bsubtype_A | TGGGGTTCGCGTA | NS5B sequence- | Amplification |
| | | TGATACCCGCTGC | based subtyping | |
| | | TTTGA | assay | |
| 2 | NS5Bsubtype_B | TGGGGTTTTCTTA | NS5B sequence- | Amplification |
| | | CGACACCAGGTG | based subtyping | |
| | | CTTTGA | assay | |
| 3 | NS5Bsubtype_C | CCGTATGATACCC | NS5B sequence- | Amplification |
| | | GCTGCTTTGACTC | based subtyping | and |
| | | AAC | assay | sequencing |
| 4 | NS5Bsubtype_D | TCCTACGACACCA | NS5B sequence- | Amplification |
| | | GGTGCTTTGATTC | based subtyping | and |
| | | AAC | assay | sequencing |
| 5 | NS5Bsubtype_E | AATTCCTGGTCAT | NS5B sequence- | Amplification |
| | | AGCCTCCGTGAA | based subtyping | and |
| | | GACTC | assay | sequencing |
| 6 | 1b_NS3_out_F | GCGTGTGGGGAC | N-terminal 181 aa | Amplification |
| | | ATCATCTTAGG | of NS3 genotyping | |
| | | | assay | |
| 7 | 1b_NS3_out_R | GCTGCCAGTGGG | N-terminal 181 aa | Amplification |
| | | AGCGTG | of NS3 genotyping | |
| | | | assay | |
| 8 | 1b_NS3_in_F | TCATCTTAGGCCT | N-terminal 181 aa | Amplification |
| | | GCCCGTCTC | of NS3 genotyping | and |
| | | | assay | sequencing |
| 9 | 1b_NS3_in_R | GGGAGCGTGTAG | N-terminal 181 aa | Amplification |
| | | ATGGGCCAC | of NS3 genotyping | and |
| | | | assay | sequencing |
| 10 | pFK 1341 PIluc | Plasmid sequence of | Phenotyping | NA |
| | deltaNS3 7- | delta[NS3] backbone | shuttle backbone | |
| | 192 ET | | | |
| 11 | 1b_InFu_NS3_F | ATGGCGCCTATTA | Phenotyping | Amplification |
| | | CCGCCTACTCCCA | amplification | |
| | | ACAGACG | primer | |
| 12 | 1b_InFu_NS3_R | AATGTCTGCGGTA | Phenotyping | Amplification |
| | | CCGCCGGGGGGG | amplification | |
| | | ATGAGTTGTC | primer | |
| 13 | 1b_NS5B_out_F | TAGAGTCCTGGA | Polymerase | Amplification |
| | | AGGACCCGG | (NS5B) | |
| | | | genotyping assay | |
| 14 | 1b_NS5B_out_R | GGCCTGGAGTGG | Polymerase | Amplification |
| | | TTAGCTCCCC | (NS5B) | |
| | | | genotyping assay | |
| 15 | 1b_NS5B_in_F | TGGAAGGACCCG | Polymerase | Amplification |
| | | GACTACG | (NS5B) | and |
| | | | genotyping assay | sequencing |
| 16 | 1b_NS5B_in_R | GAGTGGTTAGCTC | Polymerase | Amplification |
| | | CCCGTTCA | (NS5B) | and |
| | | | genotyping assay | sequencing |
| 17 | pFKi341Luc_NS | plasmid with 1st | Phenotyping | NA |
| | 3-3'-ET-AflII | AflIIsite | shuttle backbone | |
| | | (intermediate | | |
| | | plasmid) | | |
| 18 | AflII-5A-fwd | (5'-accgtaagcgaggag | Phenotyping for | |
| | | cttaaggctagtgaggacgtc- | cloning | |
| | | 3') SDM primer | | |
| 19 | AflII-5A-rev | (5'-gacgtcctcactagcctt | Phenotyping for | |
| | | aagctcctcgcttacggt-3' | cloning | |
| | | SDM primer | | |
| 20 | pFKi341Luc_NS3- | plasmid with 2nd | Phenotyping | NA |
| | 3'-ET-2xAflII | AFLii SITE | shuttle backbone | |
| | | (intermediate | | |
| | | plasmid) | | |
| 21 | pFK_1341_PI_NS3- | Plasmid sequence of | Phenotyping | NA |
| | 3_ET_dNS5A/ | delta[NS5B] ScaI | shuttle backbone | |
| | b_5a440-5b591- | backbone | | |
| | ScaI | | | |
| 22 | XbaI-mut-fwd | (5'-ggcgccattctatccac | Phenotyping for | |
| | | tagaggatggaacc-3') | cloning | |
| | | SDM primer | | |
| 23 | XbaI-mut-rev | (5'-ggttccatcctctagtg | Phenotyping for | |
| | | gatagaatggcgcc-3') | cloning | |
| | | SDM primer | | |
| 24 | XbaI-add-fwd | (5'-gagtgctgatactggcc | Phenotyping for | |
| | | tctctgcagatcaagtctaga | cloning | |
| | | aagtccctttagtgagggtta | | |
| | | attc-3') | | |
| 25 | XbaI-add-rev | (5'-gaattaaccctcactaaa | Phenotyping for | |
| | | gggactttctagacttgatctg | cloning | |
| | | cagagaggccagtatcagc | | |
| | | actc-3') | | |
| 26 | pFKi341Luc_NS3- | Intermediate plasmid | Phenotyping | NA |
| | 3'-ET-2xAfl II- | | shuttle backbone | |
| | XbaI | | | |
| 27 | pFK_1341_PI_NS3- | Plasmid sequence of | Phenotyping | NA |
| | 3_ET_dNS5A/ | delta[NS5B] XbaI | shuttle backbone | |
| | b_5a440-5b591- | backbone | | |
| | XbaI | | | |
| 28 | 1b_NS5B_F_AflII- | AAGCGAGGAGCT | Phenotyping | Amplification |
| | Infusion | TAAGGCYRGTGA | amplification | |
| | | GGACGT | primer | |
| 29 | 1b_NS5B_R_AflII- | AGCTCCCCGTCTT | Phenotyping | Amplification |
| | Infusion | AAGTCAYCGGTT | amplification | |
| | | GGGG | primer | |
| 30 | 1a_NS3/4A_out_ | GGGACCTCACCG | Protease (NS3/4A) | Amplification |
| | R | CTCATGAT | genotyping assay | |
| 31 | 1a_NS3/4A_in_R | CTCACCGCTCATG | Protease (NS3/4A) | Amplification |
| | | ATCTTGAATGC | genotyping assay | |
| 32 | 1a_NS3/4A_out_ | CGGAGGTCATTA | Protease (NS3/4A) | Amplification |
| | F | CGTGCAAATG | genotyping assay | |
| 33 | 1a_NS3/4A_in_F | CGTGCAAATGGC | Protease (NS3/4A) | Amplification |
| | | CATCATCAAG | genotyping assay | |
| 34 | 1a_NS2_F1sb | GCGCTTACTGGCA | Protease (NS3/4A) | Sequencing |
| | | CCTATG | genotyping assay | |
| 35 | 1a_NS3_F1s | AGGCACGCCGAT | Protease (NS3/4A) | Sequencing |
| | | GTCAT | genotyping assay | |
| 36 | 1a_NS3_R2s | CGGGACCTTGGT | Protease (NS3/4A) | Sequencing |
| | | GCTCTT | genotyping assay | |
| 37 | 1a_NS3_F2s | CGGCACTGTCCTT | Protease (NS3/4A) | Sequencing |
| | | GACCA | genotyping assay | |
| 38 | 1a_NS3_R3s | GAGTCGAAGTCG | Protease (NS3/4A) | Sequencing |
| | | CCGGTA | genotyping assay | |
| 39 | 1a_NS3_F3s | CCGAGACTACAG | Protease (NS3/4A) | Sequencing |
| | | TTAGGCTACG | genotyping assay | |
| 40 | 1a_NS3_R4s | GCATGTCATGATG | Protease (NS3/4A) | Sequencing |
| | | TATTTGGTG | genotyping assay | |
| 41 | 1a_NS4B_R1s | ACGAGGACCTTC | Protease (NS3/4A) | Sequencing |
| | | CCCAGT | and NS4B/5A | |
| | | | genotyping assay | |
| 42 | 1a_NS3_out_R | GCTGCCGGTGGG | N-terminal 181 aa | Amplification |
| | | AGCATG | of NS3 genotyping | |
| | | | assay | |
| 43 | 1a_NS3_in_R | GAGCATGCAGGT | N-terminal 181 aa | Amplification |
| | | GGGCCAC | of NS3 genotyping | and |
| | | | assay | sequencing |
| 44 | 1a_NS3_out_F | GCGGCGACATCA | N-terminal 181 aa | Amplification |
| | | TCAACGG | of NS3 genotyping | |
| | | | assay | |
| 45 | 1a_NS3_in_F | CATCAACGGCTTG | N-terminal 181 aa | Amplification |
| | | CCCGTCTC | of NS3 genotyping | and |
| | | | assay | sequencing |
| 46 | 1a_NS3_Fs_BU | GACCTTTACCTGG | N-terminal 181 aa | Sequencing |
| | | TCACGAG | of NS3 genotyping | |
| | | | assay | |
| 47 | 1a_NS4B/5A_out_ | GCTGTCCAGAACT | NS4B/5A | Amplification |
| | R | TGCAGTCTGTC | genotyping assay | |
| 48 | 1a_NS4B/5A_in_ | CCTTTGGCAAGCA | NS4B/5A | Amplification |
| | R | CTGCGTG | genotyping assay | |
| 49 | 1a_NS4B/5A_out_ | CTGCGTGGTCATA | NS4B/5A | Amplification |
| | F | GTGGGCAG | genotyping assay | |
| 50 | 1a_NS4B/5A_in_ | TGTCTTGTCCGGG | NS4B/5A | Amplification |
| | F | AAGCCGG | genotyping assay | |
| 51 | 1a_NS4B_F2s | CGTCACTGCCATA | NS4B/5A | Sequencing |
| | | CTCAGCA | genotyping assay | |
| 52 | 1a_NS5A_R1s | CGTCCCGTTTTTG | NS4B/5A | Sequencing |
| | | ACATG | genotyping assay | |
| 53 | 1a_NS5A_R2s | TGACTCAACCCTG | NS4B/5A | Sequencing |
| | | GTGATGTT | genotyping assay | |
| 54 | 1a_NS5A_F2s | CGGTGGTCCTCAC | NS4B/5A and | Sequencing |
| | | CGAA | Polymerase | |
| | | | (NS5B) | |
| | | | genotyping assay | |
| 55 | 1a_NS4A_F1s | TTGTCCGGGAAG | NS4B/5A | Sequencing |
| | | CCG | genotyping assay | |
| 56 | 1a_NS5B_R1s | TGGCAAGCACTG | NS4B/5A | Sequencing |
| | | CGTG | genotyping assay | |
| 57 | 1a_NS5A_F1s | TTGACGTCCATGC | NS4B/5A | Sequencing |
| | | TCACTG | genotyping assay | |
| 58 | 1a_NS5B_out_R | AGGCCGGAGTGT | Polymerase | Amplification |
| | | TTACCCCAAC | (NS5B) | |
| | | | genotyping assay | |
| 59 | 1a_NS5B_in_R | GGAGTGTTTACCC | Polymerase | Amplification |
| | | CAACCTTCA | (NS5B) | and |
| | | | genotyping assay | sequencing |
| 60 | 1a_NS5B_out_F | TGACTATGAACC | Polymerase | Amplification |
| | | ACCTGTGGTCC | (NS5B) | |
| | | | genotyping assay | |
| 61 | 1a_NS5B_in_F | CACCTGTGGTCCA | Polymerase | Amplification |
| | | TGGCTG | (NS5B) | and |
| | | | genotyping assay | sequencing |
| 62 | 1a_NS5B_F1s | CATCAACTCCGTG | Polymerase | Sequencing |
| | | TGGAAAG | (NS5B) | |
| | | | genotyping assay | |
| 63 | 1a_NS5B_R1s | CAGCGGGTATCA | Polymerase | Sequencing |
| | | TACGAGAA | (NS5B) | |
| | | | genotyping assay | |
| 64 | 1a_NS5B_F2s | GCACCATGCTCGT | Polymerase | Sequencing |
| | | GTGTG | (NS5B) | |
| | | | genotyping assay | |
| 65 | 1a_NS5B_R2s | GTCATCAGTATCA | Polymerase | Sequencing |
| | | TCCTCGCC | (NS5B) | |
| | | | genotyping assay | |
| 66 | 1a_NS5B_F3s | CGACTCCATGGTC | Polymerase | Sequencing |
| | | TTAGCG | (NS5B) | |
| | | | genotyping assay | |
| 67 | 1b_NS3/4A_out_ | GAGCGCCTTCTGT | Protease (NS3/4A) | Amplification |
| | R | TTGAATTG | genotyping assay | |
| 68 | 1b_NS3/4A_in_R | CTGTTTGAATTGC | Protease (NS3/4A) | Amplification |
| | | TCGGCGAG | genotyping assay | and |
| | | | | sequencing |
| 69 | 1b_NS3/4A_out_ | ATGCATGCTGGTG | Protease (NS3/4A) | Amplification |
| | F | CGGAA | genotyping assay | |
| 70 | 1b_NS3/4A_in_F | TGGTGCGGAAAG | Protease (NS3/4A) | Amplification |
| | | TCGCTGG | genotyping assay | |
| 71 | 1b_NS2_F1s | GGTCATTATGTCC | Protease (NS3/4A) | Sequencing |
| | | AAATGGC | genotyping assay | |
| 72 | 1b_NS3_F1s | CGGCAGCTCGGA | Protease (NS3/4A) | Sequencing |
| | | CCTTTA | genotyping assay | |
| 73 | 1b_NS3_R2s | CACTTGGAATGTC | Protease (NS3/4A) | Sequencing |
| | | TGCGGTAC | genotyping assay | |
| 74 | 1b_NS3_F2s | GATGAGTGCCAC | Protease (NS3/4A) | Sequencing |
| | | TCAACTGACT | genotyping assay | |
| 75 | 1b_NS3_R3s | CGTCTGTTGCCAC | Protease (NS3/4A) | Sequencing |
| | | GACAA | genotyping assay | |
| 76 | 1b_NS3_F3s | CTATGACGCGGG | Protease (NS3/4A) | Sequencing |
| | | CTGTG | genotyping assay | |
| 77 | 1b_NS3_R4s | AGCCGTATGAGA | Protease (NS3/4A) | Sequencing |
| | | CACTTCCAC | genotyping assay | |
| 78 | 1b_NS4B/5A_out_ | GCATAGACCATG | NS4B/5A | Amplification |
| | R | TTGTGGTGACG | genotyping assay | |
| 79 | 1b_NS4B/5A_in_ | GTGACGCAGCAA | NS4B/5A | Amplification |
| | R | AGAGTTGCTCA | genotyping assay | and |
| | | | | sequencing |
| 80 | 1b_NS4B/5A_out_ | AGCGTGGTCATTG | NS4B/5A | Amplification |
| | F | TGGGCAG | genotyping assay | |
| 81 | 1b_NS4B/5A_in_ | GGGCAGGATCAT | NS4B/5A | Amplification |
| | F | CTTGTCCGG | genotyping assay | and |
| | | | | sequencing |
| 82 | 1b_NS4B_R1s | TTCCCAAGGCCTA | NS4B/5A | Sequencing |
| | | TGCTG | genotyping assay | |
| 83 | 1b_NS4B_F2s | GGATGAACCGGC | NS4B/5A | Sequencing |
| | | TGATAGC | genotyping assay | |
| 84 | 1b_NS5A_R1s | ATGGAACCGTTTT | NS4B/5A | Sequencing |
| | | TGACATGT | genotyping assay | |
| 85 | 1b_NS5A_F1s | GGGCATGACCAC | NS4B/5A | Sequencing |
| | | TGACAAC | genotyping assay | |
| 86 | 1b_NS5A_R2s | CCACAGGAGGTT | NS4B/5A | Sequencing |
| | | GGCCT | genotyping assay | |
| 87 | 1b_NS5A_F2s | CACGGGTGCCCA | NS4B/5A and | Sequencing |
| | | TTGC | Polymerase | |
| | | | (NS5B) | |
| | | | genotyping assay | |
| 88 | 1b_NS5B_F1s | AAGGAGATGAAG | Polymerase | Sequencing |
| | | GCGAAGG | (NS5B) | |
| | | | genotyping assay | |
| 89 | 1b_NS5B_R1s | CATCACGGCCTG | Polymerase | Sequencing |
| | | AGGAAG | (NS5B) | |
| | | | genotyping assay | |
| 90 | 1b_NS5B_F2s | TCGCTCACAGAG | Polymerase | Sequencing |
| | | CGGCT | (NS5B) | |
| | | | genotyping assay | |
| 91 | 1b_NS5B_R2s | TGGAGGAGCATG | Polymerase | Sequencing |
| | | ATGTTATCA | (NS5B) | |
| | | | genotyping assay | |
| 92 | 1b_NS5B_F3s | CGACTCCATGGTC | Polymerase | Sequencing |
| | | TTAGCG | (NS5B) | |
| | | | genotyping assay | |
| 93 | 2a_NS3/4A in_F | GTAGGTGGACTG | Protease (NS3/4A) | Amplification |
| | | GCACTTACATCTA | genotyping assay | |
| | | TGA | | |
| 94 | 2a_NS3/4A out_F | CGCTATTAGCCCT | Protease (NS3/4A) | Amplification |
| | | TGGTAGGTGG | genotyping assay | |
| 95 | 2a_NS3/4A in_R | AAATGCCCGCAC | Protease (NS3/4A) | Amplification |
| | | CATACCC | genotyping assay | and |
| | | | | sequencing |
| 96 | 2a_NS3/4A | GGCTTCTCGCCAG | Protease (NS3/4A) | Amplification |
| | out_R | ACATGATCTT | genotyping assay | |
| 97 | 2a_NS2_F2sb | CACGGACTTCCCG | Protease (NS3/4A) | Sequencing |
| | | TGTC | genotyping assay | |
| 98 | 2a_NS3_R1sb | TGCCAGTTGGGG | Protease (NS3/4A) | Sequencing |
| | | CATG | genotyping assay | |
| 99 | 2a_NS3_F1s | TCCGGGCAGCTGT | Protease (NS3/4A) | Sequencing |
| | | GTG | genotyping assay | |
| 100 | 2a_NS3_R2s | CGTCTTGAGGGA | Protease (NS3/4A) | Sequencing |
| | | CAGTCTGTG | genotyping assay | |
| 101 | 2a_NS3_F2s | GGAGGGTGAGAT | Protease (NS3/4A) | Sequencing |
| | | CCCCTTCTA | genotyping assay | |
| 102 | 2a_NS4B_R1s | GAAGTTCCACAT | Protease (NS3/4A) | Sequencing |
| | | GTGTTTGGC | genotyping assay | |
| 103 | 2a_NS3_F3s | GTAGTGCTCTGTG | Protease (NS3/4A) | Sequencing |
| | | AGTGCTACG | genotyping assay | |
| 104 | 2a_NS3_in_F | ATCTTACACGGAC | N-terminal 181 aa | Amplification |
| | | TCCCCGTGTC | of NS3 genotyping | and |
| | | | assay | sequencing |
| 105 | 2a_NS3_out_F | ATGCGGGGACAT | N-terminal 181 aa | Amplification |
| | | CTTACACGG | of NS3 genotyping | |
| | | | assay | |
| 106 | 2a_NS3_in_R | TGGGGCATGCAA | N-terminal 181 aa | Amplification |
| | | GTACCCGAC | of NS3 genotyping | and |
| | | | assay | sequencing |
| 107 | 2a_NS3_out_R | CACTGCCAGTTGG | N-terminal 181 aa | Amplification |
| | | GGCATG | of NS3 genotyping | |
| | | | assay | |
| 108 | 2b_NS3/4A_in_F | TACGGATACCAT | Protease (NS3/4A) | Amplification |
| | | ACTTTGTGAGGGC | genotyping assay | |
| 109 | 2b_NS3/4A_out_ | TCTCTGCTACGGA | Protease (NS3/4A) | Amplification |
| | F | TACCATACTTTG | genotyping assay | |
| 110 | 2b_NS3/4A_in_R | TCCACCAGTATCT | Protease (NS3/4A) | Amplification |
| | | TACCCAGGCCTA | genotyping assay | |
| 111 | 2bNS3/4A_out_ | ACGTCCACCAGT | Protease (NS3/4A) | Amplification |
| | R | ATCTTACCCA | genotyping assay | |
| 112 | 2b_NS2_F1s | ACGAGTGTGTAC | Protease (NS3/4A) | Sequencing |
| | | CCTGGTGA | genotyping assay | |
| 113 | 2b_NS3_F1s | GACCCCTGTACCT | Protease (NS3/4A) | Sequencing |
| | | GCGG | genotyping assay | |
| 114 | 2b_NS3_R2s | GCAAGTAGCCCA | Protease (NS3/4A) | Sequencing |
| | | CCTGGTAAG | genotyping assay | |
| 115 | 2b_NS3_F2s | GCCATTCAGTGG | Protease (NS3/4A) | Sequencing |
| | | ACGCCAC | genotyping assay | |
| 116 | 2b_NS3_R3s | CCTTGAGTTGGTA | Protease (NS3/4A) | Sequencing |
| | | TAACGGAGAC | genotyping assay | |
| 117 | 2b_NS3_F3s | GCTCTGTGAGTGC | Protease (NS3/4A) | Sequencing |
| | | TATGATGC | genotyping assay | |
| 118 | 2b_NS3_R4s | GGTAGGACCAGT | Protease (NS3/4A) | Sequencing |
| | | CAGTGTAGGTTT | genotyping assay | |
| 119 | 2b_NS4B_R1s | CAACGAAGCCAG | Protease (NS3/4A) | Sequencing |
| | | TGGCTC | genotyping assay | |
| 120 | 2b_NS3_in_F | TGCATGGCCTCCC | N-terminal 181 aa | Amplification |
| | | GGTTTC | of NS3 genotyping | and |
| | | | assay | sequencing |
| 121 | 2b_NS3_out_F | CATGTGGAGACA | N-terminal 181 aa | Amplification |
| | | TCCTGCATGG | of NS3 genotyping | |
| | | | assay | |
| 122 | 2b_NS3_in_R | TTGGTGCATGCAA | N-terminal 181 aa | Amplification |
| | | GTAGCCCAC | of NS3 genotyping | and |
| | | | assay | sequencing |
| 123 | 2b_NS3_out_R | CGCTGCCTGTTGG | N-terminal 181 aa | Amplification |
| | | TGCATG | of NS3 genotyping | |
| | | | assay | |
| 124 | 2b_NS5B_in_F | CTTCTGTACCATC | Polymerase | Amplification |
| | | AGAGTACCTGAT | (NS5B) | and |
| | | CA | genotyping assay | sequencing |
| 125 | 2b_NS5B_out_F | GTGAGCCTTCTGT | Polymerase | Amplification |
| | | ACCATCAGAGTA | (NS5B) | |
| | | C | genotyping assay | |
| 126 | 2b_NS5B_out_R | ATGGAGTGTAGC | Polymerase | Amplification |
| | | TAGGGTTTGCC | (NS5B) | |
| | | | genotyping assay | |
| 127 | 2b_NS5B_R_in | TGTAGCTAGGGTT | Polymerase | Amplification |
| | | TGCCGCTCTA | (NS5B) | and |
| | | | genotyping assay | sequencing |
| 128 | 2b_NSSA_F2s | GAACCACCCACT | Polymerase | Sequencing |
| | | GTCCTAGG | (NS5B) | |
| | | | genotyping assay | |
| 129 | 2b_NS5B_F1s | GCACACTATGACT | Polymerase | Sequencing |
| | | CAGTCTTGCA | (NS5B) | |
| | | | genotyping assay | |
| 130 | 2b_NS5B_R1s | CATCTTTTCGCAC | Polymerase | Sequencing |
| | | ACCCTG | (NS5B) | |
| | | | genotyping assay | |
| 131 | 2b_NS5B_F2s | TACGTAGGAGGG | Polymerase | Sequencing |
| | | CCCATG | (NS5B) | |
| | | | genotyping assay | |
| 132 | 2b_NS5B_R2s | AGCGCTACCGAT | Polymerase | Sequencing |
| | | ACGTTTG | (NS5B) | |
| | | | genotyping assay | |
| 133 | 2b_NS5B_F3s | CCGGCCATAATTG | Polymerase | Sequencing |
| | | AAAGG | (NS5B) | |
| | | | genotyping assay | |
| 134 | 3a_NS3/4A_in_F | ATGCTCGTGCGCT | Protease (NS3/4A) | Amplification |
| | | CCGTGAT | genotyping assay | |
| 135 | 3aNS3/4A_out_ | CTTTGCATGCTCG | Protease (NS3/4A) | Amplification |
| | F | TGCGCTC | genotyping assay | |
| 136 | 3a_NS3/4A_in_R | TACTATGGGCTCA | Protease (NS3/4A) | Amplification |
| | | ATGACAGCTTGTT | genotyping assay | and |
| | | G | | sequencing |
| 137 | 3a_NS3/4A_out_ | GGTAGCTACTATG | Protease (NS3/4A) | Amplification |
| | R | GGCTCAATGACA | genotyping assay | |
| | | GC | | |
| 138 | 3a_NS2_F1s | TACTTCCAGATGA | Protease (NS3/4A) | Sequencing |
| | | TCATACTGAGC | genotyping assay | |
| 139 | 3a_NS3_F1s | ACTTATACTTGGT | Protease (NS3/4A) | Sequencing |
| | | TACCCGCG | genotyping assay | |
| 140 | 3a_NS3_R2s | TCTTACCGCTGCC | Protease (NS3/4A) | Sequencing |
| | | GGTC | genotyping assay | |
| 141 | 3a_NS3_F2s | TCTTAGATCAGGC | Protease (NS3/4A) | Sequencing |
| | | TGAGACGG | genotyping assay | |
| 142 | 3a_NS3_R3s | CTGTTGTTGGTAT | Protease (NS3/4A) | Sequencing |
| | | GACGGACA | genotyping assay | |
| 143 | 3a_NS3_F3s | AGCCCGCTGAGA | Protease (NS3/4A) | Sequencing |
| | | CCACA | genotyping assay | |
| 144 | 3a_NS3_R4s | ATGTAGTGTTGGC | Protease (NS3/4A) | Sequencing |
| | | TTAAGCCG | genotyping assay | |
| 145 | 3a_NS3_out_R | CTGCCGGTCGGG | N-terminal 181 aa | Amplification |
| | | GCATG | of NS3 genotyping | |
| | | | assay | |
| 146 | 3a_NS3_in_R | GGTCGGGGCATG | N-terminal 181 aa | Amplification |
| | | AAGGTATCCTAC | of NS3 genotyping | and |
| | | | assay | sequencing |
| 147 | 3a_NS3_out_F | CTTGCGGAGATAT | N-terminal 181 aa | Amplification |
| | | TCTTTGCGG | of NS3 genotyping | |
| | | | assay | |
| 148 | 3a_NS3_in_F | TTGCGGGCTGCCC | N-terminal 181 aa | Amplification |
| | | GTCTC | of NS3 genotyping | and |
| | | | assay | sequencing |
| 149 | 3a_NS4B/5A_out_ | CGACGTTGAATA | NS4B/5A | Amplification |
| | R | GACTAGGTTATG | genotyping assay | |
| | | ATGTCT | | |
| 150 | 3a_NS4B/5A_out_ | CCCTAGCGGCCTA | NS4B/5A | Amplification |
| | F | CTGCTTG | genotyping assay | |
| 151 | 3a_NS4B/5A_in_ | GGCCTACTGCTTG | NS4B/5A | Amplification |
| | F | TCAGTCGG | genotyping assay | |
| 152 | 3a_NS4A_F1s | GCCTACTGCTTGT | NS4B/5A | Sequencing |
| | | CAGTCGG | genotyping assay | |
| 153 | 3a_NS4B_R1s | ATACCCCCTATGG | NS4B/5A | Sequencing |
| | | CAGCG | genotyping assay | |
| 154 | 3a_NS4B_F2s | ACAGTGGATGAA | NS4B/5A | Sequencing |
| | | CAGGCTCAT | genotyping assay | |
| 155 | 3a_NS5A_R1s | TGACAGGAAATG | NS4B/5A | Sequencing |
| | | AAGGGCAG | genotyping assay | |
| 156 | 3a_NS5A_F1s | TGAAGTGGATGG | NS4B/5A | Sequencing |
| | | GGTGAGA | genotyping assay | |
| 157 | 3a_NS5A_R2s | TGAGGCCTATGC | NS4B/5A | Sequencing |
| | | GTCTGG | genotyping assay | |
| 158 | 3a_NS5A_F2s | CACCAACTGTCG | NS4B/5A and | Sequencing |
| | | ATGGATG | Polymerase | |
| | | | (NS5B) | |
| | | | genotyping assay | |
| 159 | 3a_NS4B/5A_in_ | TTATGATGTCTCA | NS4B/5A | Amplification |
| | R | ACAAGGAGTTGC | genotyping assay | and |
| | | TGA | | sequencing |
| 160 | 3a_NS5B_out_R | AGTGTTATCTTAC | Polymerase | Amplification |
| | | CAGCTCACCGAG | (NS5B) | |
| | | C | genotyping assay | |
| 161 | 3a_NS5B_in_R | ATCTTACCAGCTC | Polymerase | Amplification |
| | | ACCGAGCTGGC | (NS5B) | and |
| | | | genotyping assay | sequencing |
| 162 | 3a_NS5B_out_F | GTATCCTCCAGCC | Polymerase | Amplification |
| | | CTTCCTATCTG | (NS5B) | |
| | | | genotyping assay | |
| 163 | 3a_NS5B_in_F | CAGCCCTTCCTAT | Polymerase | Amplification |
| | | CTGGGCTAG | (NS5B) | and |
| | | | genotyping assay | sequencing |
| 164 | 3a_NS5B_F1s | TCGGGTATAGTGC | Polymerase | Sequencing |
| | | GAAGGA | (NS5B) | |
| | | | genotyping assay | |
| 165 | 3a_NS5B_R1s | CTTCAGCAGACGT | Polymerase | Sequencing |
| | | TCGACC | (NS5B) | |
| | | | genotyping assay | |
| 166 | 3a_NS5B_F2s | TACATCAAGGCC | Polymerase | Sequencing |
| | | ACAGCG | (NS5B) | |
| | | | genotyping assay | |
| 167 | 3a_NS5B_R2s | CTGGAGTGTGAC | Polymerase | Sequencing |
| | | GAGCTGTT | (NS5B) | |
| | | | genotyping assay | |
| 168 | 3a_NS5B_F3s | CTTGGAGACATC | Polymerase | Sequencing |
| | | GGGCAC | (NS5B) | |
| | | | genotyping assay | |
| 169 | 4a/d_NS3/4A_in_ | GCGCGTCCCTTAC | Protease (NS3/4A) | Amplification |
| | F | TTCGTGAG | genotyping assay | |
| 170 | 4a/d_NS3/4A_out_ | GCTCCTGCGCGTC | Protease (NS3/4A) | Amplification |
| | F | CCTTAC | genotyping assay | |
| 171 | 4a/d_NS3/4A_in_ | GTAGCCAGCGAG | Protease (NS3/4A) | Amplification |
| | R | GATGTCCACTAG | genotyping assay | and |
| | | | | sequencing |
| 172 | 4a/d_NS3/4A_out_ | CATCTCGCCGCTC | Protease (NS3/4A) | Amplification |
| | R | ATGATCTT | genotyping assay | |
| 173 | 4a/d_NS2_F1s | GCGTCCCTTACTT | Protease (NS3/4A) | Sequencing |
| | | CGTGAG | genotyping assay | |
| 174 | 4a/d_NS3_F1s | CCGTGCGCAGGA | Protease (NS3/4A) | Sequencing |
| | | GAGG | genotyping assay | |
| 175 | 4a/d_NS3_F2s | CACGGTCTTGGAC | Protease (NS3/4A) | Sequencing |
| | | CAAGC | genotyping assay | |
| 176 | 4a/d_NS3_F3s | GCCTGGTACGAA | Protease (NS3/4A) | Sequencing |
| | | CTGACACC | genotyping assay | |
| 177 | 4a/d_NS3_R2s | GCCACTTCCTGTT | Protease (NS3/4A) | Sequencing |
| | | GGTGC | genotyping assay | |
| 178 | 4a/d_NS3_R3s | CTGAGTCAAAGT | Protease (NS3/4A) | Sequencing |
| | | CGCCGGT | genotyping assay | |
| 179 | 4a/d_NS3_R4s | GACATGCAGGCC | Protease (NS3/4A) | Sequencing |
| | | ATGATGTA | genotyping assay | |
| 180 | 4a/d_NS3_in_F | TAAGGGGATTAC | N-terminal 181 aa | Amplification |
| | | CTGTCTCGGC | of NS3 genotyping | and |
| | | | assay | sequencing |
| 181 | 4a/d_NS3_out_F | AGTTGTGTTCACG | N-terminal 181 aa | Amplification |
| | | CCCATGGAG | of NS3 genotyping | |
| | | | assay | |
| 182 | 4a/d_NS3_in_R | GGGACTTTGGTGC | N-terminal 181 aa | Amplification |
| | | TCTTGCC | of NS3 genotyping | and |
| | | | assay | sequencing |
| 183 | 4a/d_NS3_out_R | TCGATGCCATATG | N-terminal 181 aa | Amplification |
| | | CCTTGGAC | of NS3 genotyping | |
| | | | assay | |
| 184 | 4a/d_NS4B/5A_ | TTTCAGTGGGCAG | NS4B/5A | Amplification |
| | out_F | CGTGGT | genotyping assay | |
| 185 | 4a/d_NS4B/5A_ | AGCGTGGTGATC | NS4B/5A | Amplification |
| | in_F | GTCGGGAG | genotyping assay | and |
| | | | | sequencing |
| 186 | 4a/d_NS4B/5A_ | CCTGCAGGCGGT | NS4B/5A | Amplification |
| | out_R | CGAAGG | genotyping assay | |
| 187 | 4a/d_NS4B/5A_ | CGAAGGTCACCTT | NS4B/5A | Amplification |
| | in_R | CTTCTGCCG | genotyping assay | and |
| | | | | sequencing |
| 188 | 4a/d_NS4B_R1s | AGACATGAGGGA | NS4B/5A | Sequencing |
| | | AGCAATGG | genotyping assay | |
| 189 | 4a/d_NS4B_F1sb | TGTGCAGTGGAT | NS4B/5A | Sequencing |
| | | GAACCG | genotyping assay | |
| 190 | 4a/d_NS5A_R1s | ACTCTGCGAACCT | NS4B/5A | Sequencing |
| | | CCACG | genotyping assay | |
| 191 | 4a/d_NS5A_F1s | GTTGACAGACCC | NS4B/5A | Sequencing |
| | | ATCACACAT | genotyping assay | |
| 192 | 4a/d_NS5A_R2s | TCGTCTGTCTCAA | NS4B/5A | Sequencing |
| | | CCCTGGT | genotyping assay | |
| 193 | 4a/d_NS5A_F2sb | TCTTACTCGTCAA | NS4B/5A | Sequencing |
| | | TGCCTCC | genotyping assay | |
| 194 | 4a/d_NS5B_out_ | CGGGGTAACACA | Polymerase | Amplification |
| | F | AGATAACATCAA | (NS5B) | |
| | | G | genotyping assay | |
| 195 | 4a/d_NS5B_out_ | ACCCTAAGGTCG | Polymerase | Amplification |
| | R | GAGTGTTAAGCT | (NS5B) | |
| | | | genotyping assay | |
| 196 | 4a/d_NS5B_in_F | ACAAGATAACAT | Polymerase | Amplification |
| | | CAAGTGCCCCTG | (NS5B) | |
| | | | genotyping assay | |
| 197 | 4a/d_NS5B_in_R | AAGGTCGGAGTG | Polymerase | Amplification |
| | | TTAAGCTGCCTA | (NS5B) | and |
| | | | genotyping assay | sequencing |
| 198 | 4a/d_NS5A_F2sc | CTTATTCGTCAAT | Polymerase | Sequencing |
| | | GCCTCCAC | (NS5B) | |
| | | | genotyping assay | |
| 199 | 4a/d_NS5B_F1s | ATCATGGCCAAA | Polymerase | Sequencing |
| | | AATGAGGT | (NS5B) | |
| | | | genotyping assay | |
| 200 | 4a/d_NS5B_F2s | GCCTTCACGGAG | Polymerase | Sequencing |
| | | GCTATGAC | (NS5B) | |
| | | | genotyping assay | |
| 201 | 4a/d_NS5B_F3bs | TGTGGCATATACC | Polymerase | Sequencing |
| | | TCTTTAACTGG | (NS5B) | |
| | | | genotyping assay | |
| 202 | 4a/d_NS5B_R2s | GGAGTCAAAGCA | Polymerase | Sequencing |
| | | GCGGG | (NS5B) | |
| | | | genotyping assay | |
| 203 | 4a/d_NS5B_R3s | CAGGAATTGACT | Polymerase | Sequencing |
| | | GGAGTGTGTC | (NS5B) | |
| | | | genotyping assay | |
| 204 | 4a/d_NS5B_R4s | GCACAGGAGTAA | Polymerase | Sequencing |
| | | ATAGCGGG | (NS5B) | |
| | | | genotyping assay | |

1. (canceled)

2. (canceled)

3. (canceled)

4. Vector pFK I341 PI luc NS3 7 192_ET (SEQ ID NO.10) comprising the HCV genome with a deletion spanning the HCV NS3 N-terminal 181 amino acid region.

5. Vector pFK_I341_PI_NS3-3_ET_dNS5a/b_5a440-5b591-ScaI (SEQ ID NO 21) comprising the HCV genome with a deletion spanning the HCV NS5B region.

6. Vector pFK_I341_PI_NS3-3_ET_dNS5a/b_5a440-5b591-XbaI (SEQ ID NO 27) comprising the HCV genome with a deletion spanning the HCV NS5B region.

7. (canceled)

8. (canceled)

9. Primers with SEQ ID NO 1-5 for the amplification of the NS5B region of HCV obtained from a sample of an HCV-infected patient.

10. (canceled)

11. (canceled)

12. (canceled)

13. (canceled)


SEQUENCE LISTING (228 sequences)

SEQ ID NO: 1 - DNA, 31 bp, Artificial Sequence
  Description of Artificial Sequence Synthetic primer
tggggttcgcgtatgatacccgctgctttga

SEQ ID NO: 2 - DNA, 31 bp, Artificial Sequence
  Description of Artificial Sequence Synthetic primer
tggggttttcttacgacaccaggtgctttga

SEQ ID NO: 3 - DNA, 29 bp, Artificial Sequence
  Description of Artificial Sequence Synthetic primer
ccgtatgatacccgctgctttgactcaac

SEQ ID NO: 4 - DNA, 29 bp, Artificial Sequence
  Description of Artificial Sequence Synthetic primer
tcctacgacaccaggtgctttgattcaac

SEQ ID NO: 5 - DNA, 30 bp, Artificial Sequence
  Description of Artificial Sequence Synthetic primer
aattcctggtcatagcctccgtgaagactc

SEQ ID NO: 6 - DNA, 23 bp, Artificial Sequence
  Description of Artificial Sequence Synthetic primer
gcgtgtggggacatcatcttagg

SEQ ID NO: 7 - DNA, 18 bp, Artificial Sequence
  Description of Artificial Sequence Synthetic primer
gctgccagtgggagcgtg

SEQ ID NO: 8 - DNA, 22 bp, Artificial Sequence
  Description of Artificial Sequence Synthetic primer
tcatcttaggcctgcccgtctc

SEQ ID NO: 9 - DNA, 21 bp, Artificial Sequence
  Description of Artificial Sequence Synthetic primer
gggagcgtgtagatgggccac

SEQ ID NO: 10 - DNA, 12003 bp, Hepatitis C virus
ctccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcgcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaaggaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactagccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattaccgcggtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgaacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactatagccagcccccgattgggggcgaca

SEQ ID NO: 11 - DNA, 33 bp, Artificial Sequence
  Description of Artificial Sequence Synthetic primer
atggcgcctattaccgcctactcccaacagacg

SEQ ID NO: 12 - DNA, 35 bp, Artificial Sequence
  Description of Artificial Sequence Synthetic primer
aatgtctgcggtaccgccgggggggatgagttgtc

SEQ ID NO: 13 - DNA, 21 bp, Artificial Sequence
  Description of Artificial Sequence Synthetic primer
tagagtcctggaaggacccgg

SEQ ID NO: 14 - DNA, 22 bp, Artificial Sequence
  Description of Artificial Sequence Synthetic primer
ggcctggagtggttagctcccc

SEQ ID NO: 15 - DNA, 19 bp, Artificial Sequence
  Description of Artificial Sequence Synthetic primer
tggaaggacccggactacg

SEQ ID NO: 16 - DNA, 21 bp, Artificial Sequence
  Description of Artificial Sequence Synthetic primer
gagtggttagctccccgttca

SEQ ID NO: 17 - DNA, 12573 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcaggcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgacttaagacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactata

SEQ ID NO: 18 - DNA, 36 bp, Artificial Sequence
  Description of Artificial Sequence Synthetic primer
accgtaagcgaggagcttaaggctagtgaggacgtc

SEQ ID NO: 19 - DNA, 36 bp, Artificial Sequence
  Description of Artificial Sequence Synthetic primer
gacgtcctcactagccttaagctcctcgcttacggt

SEQ ID NO: 20 - DNA, 12579 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcaggcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggagcttaaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgacttaagacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactata

SEQ ID NO: 21 - DNA, 10773 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcaggcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggccccttcgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggagcttaagacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtactactagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactata

SEQ ID NO: 22 - DNA, 31 bp, Artificial Sequence
  Description of Artificial Sequence Synthetic primer
ggcgccattctatccactagaggatggaacc

SEQ ID NO: 23 - DNA, 31 bp, Artificial Sequence
  Description of Artificial Sequence Synthetic primer
ggttccatcctctagtggatagaatggcgcc

SEQ ID NO: 24 - DNA, 63 bp, Artificial Sequence
  Description of Artificial Sequence Synthetic primer
gagtgctgatactggcctctctgcagatcaagtctagaaagtccctttagtgagggttaattc

SEQ ID NO: 25 - DNA, 63 bp, Artificial Sequence
  Description of Artificial Sequence Synthetic primer
gaattaaccctcactaaagggactttctagacttgatctgcagagaggccagtatcagcactc

SEQ ID NO: 26 - DNA, 12579 bp, Hepatitis C virus
gccagcccccgattgggggcgacactccaccatagatcactcccctgtgaggaactactgtcttcacgcagaaagcgtctagccatggcgttagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggtactgcctgatagggtgcttgcgagtgccccgggaggtctcgtagaccgtgcaccgtttaaacccccgtgctgctggaagtcgatttcaggcttagggtaaccgtggacctcgaaaacagacgcacaaaaccaagttcaatagaagggggtacaaaccagtaccaccacgaacaagcacttctgtttccccggtgatgtcgtatagactgcttgcgtggttgaaagcgacggatccgttatccgcttatgtacttcgagaagcccagtaccacctcggaatcttcgatgcgttgcgctcagcactcaaccccagagtgtagcttaggctgatgagtctggacatccctcaccggtgacggtggtccaggctgcgttggcggcctacctatggctaacgccatgggacgctagttgtgaacaaggtgtgaagagcctattgagctacataagaatcctccggcccctgaatgcggctaatcccaacctcggagcaggtggtcacaaaccagtgattggcctgtcgtaacgcgcaagtccgtggcggaaccgactactttgggtgtccgtgtttccttttattttattgtggctgcttatggtgacaatcacagattgttatcataaagcgaattggattggccatccggtgaaagtgagactcattatctatctgtttgctggatccgctccattgagtgtgtttactctaagtacaatttcaacagttatttcaatcagacaattgtatcataatggcgggcccagaagacgccaaaaacataaagaaaggcccggcgccattctatccactagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaagcggccgcgttgttaaacagaccacaacggtttccctctagcgggatcaattccgccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgataataccatggcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatgggaaccactatgcggtccccggtcttcacggacaactcgtcccctccggccgtaccgcagacattccaggtggcccatctacacgcccctactggtagcggcaagagcactaaggtgccggctgcgtatgcagcccaagggtataaggtgcttgtcctgaacccgtccgtcgccgccaccctaggtttcggggcgtatatgtctaaggcacatggtatcgaccctaacatcagaatcggggtaaggaccatcaccacgggtgcccccatcacgtactccacctatggcaagtttcttgccgacggtggttgctctgggggcgcctatgacatcataatatgtgatgagtgccactcaactgactcgaccactatcctgggcatcggcacagtcctggaccaagcggagacggctggagcgcgactcgtcgtgctcgccaccgctacgcctccgggatcggtcaccgtgccacatccaaacatcgaggaggtggctctgtccagcactggagaaatccccttttatggcaaagccatccccatcgagaccatcaagggggggaggcacctcattttctgccattccaagaagaaatgtgatgagctcgccgcgaagctgtccggcctcggactcaatgctgtagcatattaccggggccttgatgtatccgtcataccaactagcggagacgtcattgtcgtagcaacggacgctctaatgacgggctttaccggcgatttcgactcagtgatcgactgcaatacatgtgtcacccagacagtcgacttcagcctggacccgaccttcaccattgagacgacgaccgtgccacaagacgcggtgtcacgctcgcagcggcgaggcaggactggtaggggcaggatgggcatttacaggtttgtgactccaggagaacggccctcgggcatgttcgattcctcggttctgtgcgagtgctatgacgcgggctgtgcttggtacgagctcacgcccgccgagacctcagttaggttgcgggcttacctaaacacaccagggttgcccgtctgccaggaccatctggagttctgggagagcgtctttacaggcctcacccacatagacgcccatttcttgtcccagactaagcaggcaggagacaacttcccctacctggtagcataccaggctacggtgtgcgccagggctcaggctccacctccatcgtgggaccaaatgtggaagtgtctcatacggctaaagcctacgctgcacgggccaacgcccctgctgtataggctgggagccgttcaaaacgaggttactaccacacaccccataaccaaatacatcatggcatgcatgtcggctgacctggaggtcgtcacgagcacctgggtgctggtaggcggagtcctagcagctctggccgcgtattgcctgacaacaggcagcgtggtcattgtgggcaggatcatcttgtccggaaagccggccatcattcccgacagggaagtcctttaccgggagttcgatgagatggaagagtgcgcctcacacctcccttacatcgaacagggaatgcagctcgccgaacaattcaaacagaaggcaatcgggttgctgcaaacagccaccaagcaagcggaggctgctgctcccgtggtggaatccaagtggcggaccctcgaagccttctgggcgaagcatatgtggaatttcatcagcgggatacaatatttagcaggcttgtccactctgcctggcaaccccgcgatagcatcactgatggcattcacagcctctatcaccagcccgctcaccacccaacataccctcctgtttaacatcctggggggatgggtggccgcccaacttgctcctcccagcgctgcttctgctttcgtaggcgccggcatcgctggagcggctgttggcagcataggccttgggacggtgcttgtggatattttggcaggttatggagcaggggtggcaggcgcgctcgtggcctttaaggtcatgagcggcgagatgccctccaccgaggacctggttaacctactccctgctatcctctcccctggcgccctagtcgtcggggtcgtgtgcgcagcgatactgcgtcggcacgtgggcccaggggagggggctgtgcagtggatgaaccggctgatagcgttcgcttcgcggggtaaccacgtctcccccacgcactatgtgcctgagagcgacgctgcagcacgtgtcactcagatcctctctagtcttaccatcactcagctgctgaagaggcttcaccagtggatcaacgaggactgctccacgccatgctccggctcgtggctaagagatgtttgggattggatatgcacggtgttgactgatttcaagacctggctccagtccaagctcctgccgcgattgccgggagtccccttcttctcatgtcaacgtgggtacaagggagtctggcggggcgacggcatcatgcaaaccacctgcccatgtggagcacagatcaccggacatgtgaaaaacggttccatgaggatcgtggggcctaggacctgtagtaacacgtggcatggaacattccccattaacgcgtacaccacgggcccctgcacgccctccccggcgccaaattattctagggcgctgtggcgggtggctgctgaggagtacgtggaggttacgcgggtgggggatttccactacgtgacgggcatgaccactgacaacgtaaagtgcccgtgtcaggttccggcccccgaattcttcacagaagtggatggggtgcggttgcacaggtacgctccagcgtgcaaacccctcctacgggaggaggtcacattcctggtcgggctcaatcaatacctggttgggtcacagctcccatgcgagcccgaaccggacgtagcagtgctcacttccatgctcaccgacccctcccacattacggcggagacggctaagcgtaggctggccaggggatctcccccctccttggccagctcatcagctagccagctgtctgcgccttccttgaaggcaacatgcactacccgtcatgactccccggacgctgacctcatcgaggccaacctcctgtggcggcaggagatgggcgggaacatcacccgcgtggagtcagaaaataaggtagtaattttggactctttcgagccgctccaagcggaggaggatgagagggaagtatccgttccggcggagatcctgcggaggtccaggaaattccctcgagcgatgcccatatgggcacgcccggattacaaccctccactgttagagtcctggaaggacccggactacgtccctccagtggtacacgggtgtccattgccgcctgccaaggcccctccgataccacctccacggaggaagaggacggttgtcctgtcagaatctaccgtgtcttctgccttggcggagctcgccacaaagaccttcggcagctccgaatcgtcggccgtcgacagcggcacggcaacggcctctcctgaccagccctccgacgacggcgacgcgggatccgacgttgagtcgtactcctccatgcccccccttgagggggagccgggggatcccgatctcagcgacgggtcttggtctaccgtaagcgaggagcttaaggctagtgaggacgtcgtctgctgctcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatgacttaagacggggagctaaacactccaggccaataggccatcctgtttttttccctttttttttttcttttttttttttttttttttttttttttttttttctcctttttttttcctctttttttccttttctttcctttggtggctccatcttagccctagtcacggctagctgtgaaaggtccgtgagccgcttgactgcagagagtgctgatactggcctctctgcagatcaagtctagaaagtccctttagtgagggttaattcaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaagctaagctgcctgcaggtaatacgactcactata

SEQ ID NO: 27 - DNA, 12579 bp, Hepatitis C virus; identical to SEQ ID NO: 26

SEQ ID NO: 28 - DNA, 30 bp, Artificial Sequence
  Description of Artificial Sequence Synthetic primer
aagcgaggagcttaaggcyrgtgaggacgt

SEQ ID NO: 29 - DNA, 29 bp, Artificial Sequence
  Description of Artificial Sequence Synthetic primer
agctccccgtcttaagtcaycggttgggg

SEQ ID NO: 30 - DNA, 20 bp, Artificial Sequence
  Description of Artificial Sequence Synthetic primer
gggacctcaccgctcatgat

SEQ ID NO: 31 - DNA, 24 bp, Artificial Sequence
  Description of Artificial Sequence Synthetic primer
ctcaccgctcatgatcttgaatgc

SEQ ID NO: 32 - DNA, 22 bp, Artificial Sequence
  Description of Artificial Sequence Synthetic primer
cggaggtcattacgtgcaaatg

SEQ ID NO: 33 - DNA, 22 bp, Artificial Sequence
  Description of Artificial Sequence Synthetic primer
cgtgcaaatggccatcatcaag

SEQ ID NO: 34 - DNA, 19 bp, Artificial Sequence
  Description of Artificial Sequence Synthetic primer
gcgcttactggcacctatg

SEQ ID NO: 35 - DNA, 17 bp, Artificial Sequence
  Description of Artificial Sequence Synthetic primer
aggcacgccgatgtcat

SEQ ID NO: 36 - DNA, 18 bp, Artificial Sequence
  Description of Artificial Sequence Synthetic primer
cgggaccttggtgctctt

SEQ ID NO: 37 - DNA, 18 bp, Artificial Sequence
  Description of Artificial Sequence Synthetic primer
cggcactgtccttgacca

SEQ ID NO: 38 - DNA, 18 bp, Artificial Sequence
  Description of Artificial Sequence Synthetic primer
gagtcgaagtcgccggta

SEQ ID NO: 39 - DNA, 22 bp, Artificial Sequence
  Description of Artificial Sequence Synthetic primer
ccgagactacagttaggctacg

SEQ ID NO: 40 - DNA, 22 bp, Artificial Sequence
  Description of Artificial Sequence Synthetic primer
gcatgtcatgatgtatttggtg

SEQ ID NO: 41 - DNA, 18 bp, Artificial Sequence
  Description of Artificial Sequence Synthetic primer
acgaggaccttccccagt

SEQ ID NO: 42 - DNA, 18 bp, Artificial Sequence
  Description of Artificial Sequence Synthetic primer
gctgccggtgggagcatg

SEQ ID NO: 43 - DNA, 19 bp, Artificial Sequence
  Description of Artificial Sequence Synthetic primer
gagcatgcaggtgggccac

SEQ ID NO: 44 - DNA, 19 bp, Artificial Sequence
  Description of Artificial Sequence Synthetic primer
gcggcgacatcatcaacgg

SEQ ID NO: 45 - DNA, 21 bp, Artificial Sequence
  Description of Artificial Sequence Synthetic primer
catcaacggcttgcccgtctc

SEQ ID NO: 46 - DNA, 20 bp, Artificial Sequence
  Description of Artificial Sequence Synthetic primer
gacctttacctggtcacgag

SEQ ID NO: 47 - DNA, 24 bp, Artificial Sequence
  Description of Artificial Sequence Synthetic primer
gctgtccagaacttgcagtctgtc

SEQ ID NO: 48 - DNA, 20 bp, Artificial Sequence
  Description of Artificial Sequence Synthetic primer
cctttggcaagcactgcgtg

SEQ ID NO: 49 - DNA, 21 bp, Artificial Sequence
  Description of Artificial Sequence Synthetic primer
ctgcgtggtcatagtgggcag

SEQ ID NO: 50 - DNA, 20 bp, Artificial Sequence
  Description of Artificial Sequence Synthetic primer
tgtcttgtccgggaagccgg

SEQ ID NO: 51 - DNA, 20 bp, Artificial Sequence
  Description of Artificial Sequence Synthetic primer
cgtcactgccatactcagca

SEQ ID NO: 52 - DNA, 18 bp, Artificial Sequence
  Description of Artificial Sequence Synthetic primer
cgtcccgtttttgacatg

SEQ ID NO: 53 - DNA, 21 bp, Artificial Sequence
  Description of Artificial Sequence Synthetic primer
tgactcaaccctggtgatgtt

SEQ ID NO: 54 - DNA, 17 bp, Artificial Sequence
  Description of Artificial Sequence Synthetic primer
cggtggtcctcaccgaa

SEQ ID NO: 55 - DNA, 15 bp, Artificial Sequence
  Description of Artificial Sequence Synthetic primer
ttgtccgggaagccg

SEQ ID NO: 56 - DNA, 16 bp, Artificial Sequence
  Description of Artificial Sequence Synthetic primer
tggcaagcactgcgtg

SEQ ID NO: 57 - DNA, 19 bp, Artificial Sequence
  Description of Artificial Sequence Synthetic primer
ttgacgtccatgctcactg

SEQ ID NO: 58 - DNA, 22 bp, Artificial Sequence
  Description of Artificial Sequence Synthetic primer
aggccggagtgtttaccccaac

SEQ ID NO: 59 - DNA, 22 bp, Artificial Sequence
  Description of Artificial Sequence Synthetic primer
ggagtgtttaccccaaccttca

SEQ ID NO: 60 - DNA, 23 bp, Artificial Sequence
  Description of Artificial Sequence Synthetic primer
tgactatgaaccacctgtggtcc

SEQ ID NO: 61 - DNA, 19 bp, Artificial Sequence
  Description of Artificial Sequence Synthetic primer
cacctgtggtccatggctg

SEQ ID NO: 62 - DNA, 20 bp, Artificial Sequence
  Description of Artificial Sequence Synthetic primer
catcaactccgtgtggaaag

SEQ ID NO: 63 - DNA, 20 bp, Artificial Sequence
  Description of Artificial Sequence Synthetic primer
cagcgggtatcatacgagaa

SEQ ID NO: 64 - DNA, 18 bp, Artificial Sequence
  Description of Artificial Sequence Synthetic primer
gcaccatgctcgtgtgtg

SEQ ID NO: 65 - DNA, 21 bp, Artificial Sequence
  Description of Artificial Sequence Synthetic primer
gtcatcagtatcatcctcgcc

SEQ ID NO: 66 - DNA, 19 bp, Artificial Sequence
  Description of Artificial Sequence Synthetic primer
cgactccatggtcttagcg

SEQ ID NO: 67 - DNA, 21 bp, Artificial Sequence
  Description of Artificial Sequence Synthetic primer
gagcgccttctgtttgaattg

SEQ ID NO: 68 - DNA, 21 bp, Artificial Sequence
  Description of Artificial Sequence Synthetic primer
ctgtttgaattgctcggcgag

SEQ ID NO: 69 - DNA, 18 bp, Artificial Sequence
  Description of Artificial Sequence Synthetic primer
atgcatgctggtgcggaa

SEQ ID NO: 70 - DNA, 19 bp, Artificial Sequence
  Description of Artificial Sequence Synthetic primer
tggtgcggaaagtcgctgg

SEQ ID NO: 71 - DNA, 20 bp, Artificial Sequence
  Description of Artificial Sequence Synthetic primer
ggtcattatgtccaaatggc

SEQ ID NO: 72 - DNA, 18 bp, Artificial Sequence
  Description of Artificial Sequence Synthetic primer
cggcagctcggaccttta

SEQ ID NO: 73 - DNA, 21 bp, Artificial Sequence
  Description of Artificial Sequence Synthetic primer
cacttggaatgtctgcggtac

SEQ ID NO: 74 - DNA, 22 bp, Artificial Sequence
  Description of Artificial Sequence Synthetic primer
gatgagtgccactcaactgact

SEQ ID NO: 75 - DNA, 18 bp, Artificial Sequence
  Description of Artificial Sequence Synthetic primer
cgtctgttgccacgacaa

SEQ ID NO: 76 - DNA, 17 bp, Artificial Sequence
  Description of Artificial Sequence Synthetic primer
ctatgacgcgggctgtg

SEQ ID NO: 77 - DNA, 21 bp, Artificial Sequence
  Description of Artificial Sequence Synthetic primer
agccgtatgagacacttccac

SEQ ID NO: 78 - DNA, 23 bp, Artificial Sequence
  Description of Artificial Sequence Synthetic primer
gcatagaccatgttgtggtgacg

SEQ ID NO: 79 - DNA, 23 bp, Artificial Sequence
  Description of Artificial Sequence Synthetic primer
gtgacgcagcaaagagttgctca

SEQ ID NO: 80 - DNA, 20 bp, Artificial Sequence
  Description of Artificial Sequence Synthetic primer
agcgtggtcattgtgggcag

SEQ ID NO: 81 - DNA, 21 bp, Artificial Sequence
  Description of Artificial Sequence Synthetic primer
gggcaggatcatcttgtccgg

SEQ ID NO: 82 - DNA, 18 bp, Artificial Sequence
  Description of Artificial Sequence Synthetic primer
ttcccaaggcctatgctg

SEQ ID NO: 83 - DNA, 19 bp, Artificial Sequence
  Description of Artificial Sequence Synthetic primer
ggatgaaccggctgatagc

SEQ ID NO: 84 - DNA, 21 bp, Artificial Sequence
  Description of Artificial Sequence Synthetic primer
atggaaccgtttttgacatgt

SEQ ID NO: 85 - DNA, 19 bp, Artificial Sequence
  Description of Artificial Sequence Synthetic primer
gggcatgaccactgacaac

SEQ ID NO: 86 - DNA, 17 bp, Artificial Sequence
  Description of Artificial Sequence Synthetic primer
ccacaggaggttggcct

SEQ ID NO: 87 - DNA, 16 bp, Artificial Sequence
  Description of Artificial Sequence Synthetic primer
cacgggtgcccattgc

SEQ ID NO: 88 - DNA, 19 bp, Artificial Sequence
  Description of Artificial Sequence Synthetic primer
aaggagatgaaggcgaagg

SEQ ID NO: 89 - DNA, 18 bp, Artificial Sequence
  Description of Artificial Sequence Synthetic primer
catcacggcctgaggaag

SEQ ID NO: 90 - DNA, 17 bp, Artificial Sequence
  Description of Artificial Sequence Synthetic primer
tcgctcacagagcggct

SEQ ID NO: 91 - DNA, 21 bp, Artificial Sequence
  Description of Artificial Sequence Synthetic primer
tggaggagcatgatgttatca

SEQ ID NO: 92 - DNA, 19 bp, Artificial Sequence
  Description of Artificial Sequence Synthetic primer
cgactccatggtcttagcg

SEQ ID NO: 93 - DNA, 28 bp, Artificial Sequence
  Description of Artificial Sequence Synthetic primer
gtaggtggactggcacttacatctatga

SEQ ID NO: 94 - DNA, 23 bp, Artificial Sequence
  Description of Artificial Sequence Synthetic primer
cgctattagcccttggtaggtgg

SEQ ID NO: 95 - DNA, 19 bp, Artificial Sequence
  Description of Artificial Sequence Synthetic primer
aaatgcccgcaccataccc

SEQ ID NO: 96 - DNA, 23 bp, Artificial Sequence
  Description of Artificial Sequence Synthetic primer
ggcttctcgccagacatgatctt

SEQ ID NO: 97 - DNA, 17 bp, Artificial Sequence
  Description of Artificial Sequence Synthetic primer
cacggacttcccgtgtc

SEQ ID NO: 98 - DNA, 16 bp, Artificial Sequence
  Description of Artificial Sequence Synthetic primer
tgccagttggggcatg

SEQ ID NO: 99 - DNA, 16 bp, Artificial Sequence
  Description of Artificial Sequence Synthetic primer
tccgggcagctgtgtg

SEQ ID NO: 100 - DNA, 21 bp, Artificial Sequence
  Description of Artificial Sequence Synthetic primer
cgtcttgagggacagtctgtg

SEQ ID NO: 101 - DNA, 21 bp, Artificial Sequence
  Description of Artificial Sequence Synthetic primer
ggagggtgagatccccttcta

SEQ ID NO: 102 - DNA, 21 bp, Artificial Sequence
  Description of Artificial Sequence Synthetic primer
gaagttccacatgtgtttggc

SEQ ID NO: 103 - DNA, 22 bp, Artificial Sequence
  Description of Artificial Sequence Synthetic primer
gtagtgctctgtgagtgctacg

SEQ ID NO: 104 - DNA, 23 bp, Artificial Sequence
  Description of Artificial Sequence Synthetic primer
atcttacacggactccccgtgtc

SEQ ID NO: 105 - DNA, 21 bp, Artificial Sequence
  Description of Artificial Sequence Synthetic primer
atgcggggacatcttacacgg

SEQ ID NO: 106 - DNA, 21 bp, Artificial Sequence
  Description of Artificial Sequence Synthetic primer
tggggcatgcaagtacccgac

SEQ ID NO: 107 - DNA, 19 bp, Artificial Sequence
  Description of Artificial Sequence Synthetic primer
cactgccagttggggcatg

SEQ ID NO: 108 - DNA, 25 bp, Artificial Sequence
  Description of Artificial Sequence Synthetic primer
tacggataccatactttgtgagggc

SEQ ID NO: 109 - DNA, 25 bp, Artificial Sequence
  Description of Artificial Sequence Synthetic primer
tctctgctacggataccatactttg

SEQ ID NO: 110 - DNA, 25 bp, Artificial Sequence
  Description of Artificial Sequence Synthetic primer
tccaccagtatcttacccaggccta

SEQ ID NO: 111 - DNA, 22 bp, Artificial Sequence
  Description of Artificial Sequence Synthetic primer
acgtccaccagtatcttaccca

SEQ ID NO: 112 - DNA, 20 bp, Artificial Sequence
  Description of Artificial Sequence Synthetic primer
acgagtgtgtaccctggtga

SEQ ID NO: 113 - DNA, 17 bp, Artificial Sequence
  Description of Artificial Sequence Synthetic primer
gacccctgtacctgcgg

SEQ ID NO: 114 - DNA, 21 bp, Artificial Sequence
  Description of Artificial Sequence Synthetic primer
gcaagtagcccacctggtaag

SEQ ID NO: 115 - DNA, 19 bp, Artificial Sequence
  Description of Artificial Sequence Synthetic primer
gccattcagtggacgccac

SEQ ID NO: 116 - DNA, 23 bp, Artificial Sequence
  Description of Artificial Sequence Synthetic primer
ccttgagttggtataacggagac

SEQ ID NO: 117 - DNA, 21 bp, Artificial Sequence
  Description of Artificial Sequence Synthetic primer
gctctgtgagtgctatgatgc

SEQ ID NO: 118 - DNA, 24 bp, Artificial Sequence
  Description of Artificial Sequence Synthetic primer
ggtaggaccagtcagtgtaggttt

SEQ ID NO: 119 - DNA, 18 bp, Artificial Sequence
  Description of Artificial Sequence Synthetic primer
caacgaagccagtggctc

SEQ ID NO: 120 - DNA, 19 bp, Artificial Sequence
  Description of Artificial Sequence Synthetic primer
tgcatggcctcccggtttc

SEQ ID NO: 121 - DNA, 22 bp, Artificial Sequence
  Description of Artificial Sequence Synthetic primer
catgtggagacatcctgcatgg

SEQ ID NO: 122 - DNA, 22 bp, Artificial Sequence
  Description of Artificial Sequence Synthetic primer
ttggtgcatgcaagtagcccac

SEQ ID NO: 123 - DNA, 19 bp, Artificial Sequence
  Description of Artificial Sequence Synthetic primer
cgctgcctgttggtgcatg

SEQ ID NO: 124 - DNA, 27 bp, Artificial Sequence
  Description of Artificial Sequence Synthetic primer
cttctgtaccatcagagtacctgatca

SEQ ID NO: 125 - DNA, 26 bp, Artificial Sequence
  Description of Artificial Sequence Synthetic primer
gtgagccttctgtaccatcagagtac

SEQ ID NO: 126 - DNA, 23 bp, Artificial Sequence
  Description of Artificial Sequence Synthetic primer
atggagtgtagctagggtttgcc

SEQ ID NO: 127 - DNA, 23 bp, Artificial Sequence
  Description of Artificial Sequence Synthetic primer
tgtagctagggtttgccgctcta

SEQ ID NO: 128 - DNA, 20 bp, Artificial Sequence
  Description of Artificial Sequence Synthetic primer
gaaccacccactgtcctagg

SEQ ID NO: 129 - DNA, 23 bp, Artificial Sequence
  Description of Artificial Sequence Synthetic primer
gcacactatgactcagtcttgca

SEQ ID NO: 130 - DNA, 19 bp, Artificial Sequence
  Description of Artificial Sequence Synthetic primer
catcttttcgcacaccctg

SEQ ID NO: 131 - DNA, 18 bp, Artificial Sequence
  Description of Artificial Sequence Synthetic primer
tacgtaggagggcccatg

SEQ ID NO: 132 - DNA, 19 bp, Artificial Sequence
  Description of Artificial Sequence Synthetic primer
agcgctaccgatacgtttg

SEQ ID NO: 133 - DNA, 18 bp, Artificial Sequence
  Description of Artificial Sequence Synthetic primer
ccggccataattgaaagg

SEQ ID NO: 134 - DNA, 20 bp, Artificial Sequence
  Description of Artificial Sequence Synthetic primer
atgctcgtgcgctccgtgat

SEQ ID NO: 135 - DNA, 20 bp, Artificial Sequence
  Description of Artificial Sequence Synthetic primer
ctttgcatgctcgtgcgctc

SEQ ID NO: 136 - DNA, 27 bp, Artificial Sequence
  Description of Artificial Sequence Synthetic primer
tactatgggctcaatgacagcttgttg

SEQ ID NO: 137 - DNA, 27 bp, Artificial Sequence
  Description of Artificial Sequence Synthetic primer
ggtagctactatgggctcaatgacagc

SEQ ID NO: 138 - DNA, 24 bp, Artificial Sequence
  Description of Artificial Sequence Synthetic primer
tacttccagatgatcatactgagc

SEQ ID NO: 139 - DNA, 21 bp, Artificial Sequence
  Description of Artificial Sequence Synthetic primer
acttatacttggttacccgcg

SEQ ID NO: 140 - DNA, 17 bp, Artificial Sequence
  Description of Artificial Sequence Synthetic primer
tcttaccgctgccggtc

SEQ ID NO: 141 - DNA, 21 bp, Artificial Sequence
  Description of Artificial Sequence Synthetic primer
tcttagatcaggctgagacgg

SEQ ID NO: 142 - DNA, 21 bp, Artificial Sequence
  Description of Artificial Sequence Synthetic primer
ctgttgttggtatgacggaca

SEQ ID NO: 143 - DNA, 17 bp, Artificial Sequence
  Description of Artificial Sequence Synthetic primer
agcccgctgagaccaca

SEQ ID NO: 144 - DNA, 21 bp, Artificial Sequence
  Description of Artificial Sequence Synthetic primer
atgtagtgttggcttaagccg

SEQ ID NO: 145 - DNA, 17 bp, Artificial Sequence
  Description of Artificial Sequence Synthetic primer
ctgccggtcggggcatg

SEQ ID NO: 146 - DNA, 24 bp, Artificial Sequence
  Description of Artificial Sequence Synthetic primer
ggtcggggcatgaaggtatcctac

SEQ ID NO: 147 - DNA, 22 bp, Artificial Sequence
  Description of Artificial Sequence Synthetic primer
cttgcggagatattctttgcgg

SEQ ID NO: 148 - DNA, 18 bp, Artificial Sequence
  Description of Artificial Sequence Synthetic primer
ttgcgggctgcccgtctc

SEQ ID NO: 149 - DNA, 30 bp, Artificial Sequence
  Description of Artificial Sequence Synthetic primer
cgacgttgaatagactaggttatgatgtct

SEQ ID NO: 150 - DNA, 20 bp, Artificial Sequence
  Description of Artificial Sequence Synthetic primer
ccctagcggcctactgcttg

SEQ ID NO: 151 - DNA, 21 bp, Artificial Sequence
  Description of Artificial Sequence Synthetic primer
ggcctactgcttgtcagtcgg

SEQ ID NO: 152 - DNA, 20 bp, Artificial Sequence
  Description of Artificial Sequence Synthetic primer
gcctactgcttgtcagtcgg

SEQ ID NO: 153 - DNA, 18 bp, Artificial Sequence
  Description of Artificial Sequence Synthetic primer
ataccccctatggcagcg

SEQ ID NO: 154 - DNA, 21 bp, Artificial Sequence
  Description of Artificial Sequence Synthetic primer
acagtggatgaacaggctcat

SEQ ID NO: 155 - DNA, 20 bp, Artificial Sequence
  Description of Artificial Sequence Synthetic primer
tgacaggaaatgaagggcag

SEQ ID NO: 156 - DNA, 19 bp, Artificial Sequence
  Description of Artificial Sequence Synthetic primer
tgaagtggatggggtgaga

SEQ ID NO: 157 - DNA, 18 bp, Artificial Sequence
  Description of Artificial Sequence Synthetic primer
tgaggcctatgcgtctgg

SEQ ID NO: 158 - DNA, 19 bp, Artificial Sequence
  Description of Artificial Sequence Synthetic primer
caccaactgtcgatggatg

SEQ ID NO: 159 - DNA, 28 bp, Artificial Sequence
  Description of Artificial Sequence Synthetic primer
ttatgatgtctcaacaaggagttgctga

SEQ ID NO: 160 - DNA, 26 bp, Artificial Sequence
  Description of Artificial Sequence Synthetic primer
agtgttatcttaccagctcaccgagc

SEQ ID NO: 161 - DNA, 24 bp, Artificial Sequence
  Description of Artificial Sequence Synthetic primer
atcttaccagctcaccgagctggc

SEQ ID NO: 162 - DNA, 24 bp, Artificial Sequence
  Description of Artificial Sequence Synthetic primer
gtatcctccagcccttcctatctg

SEQ ID NO: 163 - DNA, 22 bp, Artificial Sequence
  Description of Artificial Sequence Synthetic primer
cagcccttcctatctgggctag

SEQ ID NO: 164 - DNA, 19 bp, Artificial Sequence
  Description of Artificial Sequence Synthetic primer
tcgggtatagtgcgaagga

SEQ ID NO: 165 - DNA, 19 bp, Artificial Sequence
  Description of Artificial Sequence Synthetic primer
cttcagcagacgttcgacc

SEQ ID NO: 166 - DNA, 18 bp, Artificial Sequence
  Description of Artificial Sequence Synthetic primer
tacatcaaggccacagcg

SEQ ID NO: 167 - DNA, 20 bp, Artificial Sequence
  Description of Artificial Sequence Synthetic primer
ctggagtgtgacgagctgtt

SEQ ID NO: 168 - DNA, 18 bp, Artificial Sequence
  Description of Artificial Sequence Synthetic primer
cttggagacatcgggcac

SEQ ID NO: 169 - DNA, 21 bp, Artificial Sequence
  Description of Artificial Sequence Synthetic primer
gcgcgtcccttacttcgtgag

SEQ ID NO: 170 - DNA, 19 bp, Artificial Sequence
  Description of Artificial Sequence Synthetic primer
gctcctgcgcgtcccttac

SEQ ID NO: 171 - DNA, 24 bp, Artificial Sequence
  Description of Artificial Sequence Synthetic primer
gtagccagcgaggatgtccactag

SEQ ID NO: 172 - DNA, 21 bp, Artificial Sequence
  Description of Artificial Sequence Synthetic primer
catctcgccgctcatgatctt

SEQ ID NO: 173 - DNA, 19 bp, Artificial Sequence
  Description of Artificial Sequence Synthetic primer
gcgtcccttacttcgtgag

SEQ ID NO: 174 - DNA, 16 bp, Artificial Sequence
  Description of Artificial Sequence Synthetic primer
ccgtgcgcaggagagg

SEQ ID NO: 175 - DNA, 18 bp, Artificial Sequence
  Description of Artificial Sequence Synthetic primer
cacggtcttggaccaagc

SEQ ID NO: 176 - DNA, 20 bp, Artificial Sequence
  Description of Artificial Sequence Synthetic primer
gcctggtacgaactgacacc

SEQ ID NO: 177 - DNA, 18 bp, Artificial Sequence
  Description of Artificial Sequence Synthetic primer
gccacttcctgttggtgc

SEQ ID NO: 178 - DNA, 19 bp, Artificial Sequence
  Description of Artificial Sequence Synthetic primer
ctgagtcaaagtcgccggt

SEQ ID NO: 179 - DNA, 20 bp, Artificial Sequence
  Description of Artificial Sequence Synthetic primer
gacatgcaggccatgatgta

SEQ ID NO: 180 - DNA, 22 bp, Artificial Sequence
  Description of Artificial Sequence Synthetic primer
taaggggattacctgtctcggc

SEQ ID NO: 181 - DNA, 22 bp, Artificial Sequence
  Description of Artificial Sequence Synthetic primer
agttgtgttcacgcccatggag

SEQ ID NO: 182 - DNA, 20 bp, Artificial Sequence
  Description of Artificial Sequence Synthetic primer
gggactttggtgctcttgcc

SEQ ID NO: 183 - DNA, 21 bp, Artificial Sequence
  Description of Artificial Sequence Synthetic primer
tcgatgccatatgccttggac

SEQ ID NO: 184 - DNA, 19 bp, Artificial Sequence
  Description of Artificial Sequence Synthetic primer
tttcagtgggcagcgtggt

SEQ ID NO: 185 - DNA, 20 bp, Artificial Sequence
  Description of Artificial Sequence Synthetic primer
agcgtggtgatcgtcgggag

SEQ ID NO: 186 - DNA, 18 bp, Artificial Sequence
  Description of Artificial Sequence Synthetic primer
cctgcaggcggtcgaagg

SEQ ID NO: 187 - DNA, 22 bp, Artificial Sequence
  Description of Artificial Sequence Synthetic primer
cgaaggtcaccttcttctgccg

SEQ ID NO: 188 - DNA, 20 bp, Artificial Sequence
  Description of Artificial Sequence Synthetic primer
agacatgagggaagcaatgg

SEQ ID NO: 189 - DNA, 18 bp, Artificial Sequence
  Description of Artificial Sequence Synthetic primer
tgtgcagtggatgaaccg

SEQ ID NO: 190 - DNA, 18 bp, Artificial Sequence
  Description of Artificial Sequence Synthetic primer
actctgcgaacctccacg

SEQ ID NO: 191 - DNA, 21 bp, Artificial Sequence
  Description of Artificial Sequence Synthetic primer
gttgacagacccatcacacat

SEQ ID NO: 192 - DNA, 20 bp, Artificial Sequence
  Description of Artificial Sequence Synthetic primer
tcgtctgtctcaaccctggt

SEQ ID NO: 193 - DNA, 20 bp, Artificial Sequence
  Description of Artificial Sequence Synthetic primer
tcttactcgtcaatgcctcc

SEQ ID NO: 194 - DNA, 25 bp, Artificial Sequence
  Description of Artificial Sequence Synthetic primer
cggggtaacacaagataacatcaag

SEQ ID NO: 195 - DNA, 24 bp, Artificial Sequence
  Description of Artificial Sequence Synthetic primer
accctaaggtcggagtgttaagct

SEQ ID NO: 196 - DNA, 24 bp, Artificial Sequence
  Description of Artificial Sequence Synthetic primer
acaagataacatcaagtgcccctg

SEQ ID NO: 197 - DNA, 24 bp, Artificial Sequence
  Description of Artificial Sequence Synthetic primer
aaggtcggagtgttaagctgccta

SEQ ID NO: 198 - DNA, 21 bp, Artificial Sequence
  Description of Artificial Sequence Synthetic primer
cttattcgtcaatgcctccac

SEQ ID NO: 199 - DNA, 20 bp, Artificial Sequence
  Description of Artificial Sequence Synthetic primer
atcatggccaaaaatgaggt

SEQ ID NO: 200 - DNA, 20 bp, Artificial Sequence
  Description of Artificial Sequence Synthetic primer
gccttcacggaggctatgac

SEQ ID NO: 201 - DNA, 24 bp, Artificial Sequence
  Description of Artificial Sequence Synthetic primer
tgtggcatatacctctttaactgg

SEQ ID NO: 202 - DNA, 17 bp, Artificial Sequence
  Description of Artificial Sequence Synthetic primer
ggagtcaaagcagcggg

SEQ ID NO: 203 - DNA, 22 bp, Artificial Sequence
  Description of Artificial Sequence Synthetic primer
caggaattgactggagtgtgtc

SEQ ID NO: 204 - DNA, 20 bp, Artificial Sequence
  Description of Artificial Sequence Synthetic primer
gcacaggagtaaatagcggg

SEQ ID NO: 205 - DNA, 329 bp, Hepatitis C virus
agtcaccgagaatgatatccgtgttgaggagtcaatttaccaatgctgtgacttggcccccgaagccaaacaggccataaggtcgctcacagagcggctttayatcgggggtcccctgactaattcaaaagggcagaactgcggttatcgccggtgccgcgcgagcggcgtgctgacgaccagctgcggtaataccctcacctgttacttgaaggccaccgcggcctgtcgagctgcaaagctccaggactgcacgatgctcgtgtgcggggacgaccttgtcgttatctgtgaaagcgcgggaacccaagaggacgcggcgaacctac

SEQ ID NO: 206 - DNA, 329 bp, Hepatitis C virus
tgtcacygagagtgacatccgygttgaggagtcaatctaccaatgttgtgacttggcccccgaagccagacaggccataaagtcgctcacagagcggctttayatcgggggtcccctgactaaytcaaaaggrcagaactgcggytatcgccggtgccgcgcgagcggcgtgctgacgactagctgcggyaacaccctcacmtgttacytgaaggcctctgcagcctgtcgagctgcraagctccaggactgcacgatgctcgtgtgcggggacgaccttgtcgttatctgcgagagtgctgggacccaggaggacgyggcgagcctac

SEQ ID NO: 207 - DNA, 329 bp, Hepatitis C virus
ggtcactgagaatgacattcgtgtcgaggagtcgatctaccaatgctgtgacttggcccccgaagccagacargccataaggtcgctcacggagcggctttatatcgggggtcccctgactaattcaaaagggcagaactgcggttatcgccggtgccgcgcgagcggtgtactgacgaccagctgtggtaataccctcacatgttacttgaaggcctctgcggcctgtcgagctgccaagctccaggactgcacgatgctcgtgaacggagacgaccttgtcgttatctgtgagagcgcgggaacccaaraggacgcagcgaacctac

SEQ ID NO: 208 - DNA, 329 bp, Hepatitis C virus
rgtcaccgagagkgacatccgtgttgaggagtcratytaccaatgttgtgacttggcccccgaagccagacaggccataaagtcgctcacrgagcggctctatatcgggggccccctgactaattcaaaagggcagaactgcggttatcgccggtgccgcgccagcggcgtrctgacgaccagctgcggtaataccctcacatgttacttgaaggcctctgcggcctgtcgagctgcaaagctccaggactgcacgatgcttgtgtgyggagacgacctygtcgttatctgtgagagcgcggggacccaggaggacgcggcgagcctac

SEQ ID NO: 209 - DNA, 329 bp, Hepatitis C virus
ggtcactgagagtgayatccgtgtygaggagtcaatataccaatgttgtgacttggcccccgaagccagacaggccataaagtcgctcacagagcggctctatgttgggggtcccctgactaaytcaaaagggcagaactgcggttatcgccggtgccgcgccagcggcgtgctgacgaccagctgcggtaataccctcacttgttacttgaaagcctctgcrgcctgtcgagctgcgaagctccaggactgcacgatgctcgtgtgtggagacgaccttgtcgttatctgcgaaagcgcgggaacccaggaggacgcggcgagcctac

SEQ ID NO: 210 - DNA, 329 bp, Hepatitis C virus
agtcactgagagtgacatccgcgttgaggagtcaatctaccaatgttgtgacttggcccccgaagccaaacaggccataaagtcgctcacagagcggctttacatcgggggtcccctgactaattcaaaagggcagaactgcggctatcgccggtgccgcgccagcggcgtactgacgaccagctgtggtaataccctcacatgttacttgaaagcctctgcggcctgtcgagctgcaaagctccaggactgcacgatgctcgtgtgcggagacgaccttgtcgttatctgtgagagcgcgggaacccaggaggacgcggcgagcctac

SEQ ID NO: 211 - DNA, 329 bp, Hepatitis C virus
ggtcactgagagtgatatccgtactgaggagtctatttaccaatgttgtgacctggcccccgaagctagacaagtcataaggtcgctcacagagcggctttayatygggggccccctgacyaattcaaaagggcagaactgcggttatcgccggtgccgygcgagcggcgtgctgacgactagctgcggtaataccctcacatgttacttgaaggcctctgcggcctgtcgagctgcaaagctccgggactgcacgatgctcgtgtgcggagacgacctcgtcgttatctgtgaaagcgcggggacccaggaggacgcggctagcctac

SEQ ID NO: 212 - DNA, 329 bp, Hepatitis C virus; identical to SEQ ID NO: 205

SEQ ID NO: 213 - DNA, 329 bp, Hepatitis C virus
ggtcacygagagygacatccgtactgaggagtcaatttaccaatgttgtgacttggcccccgaagccagacaggttataaggtcgctcacagagcggctttatatcgggggtcctytgactaattcaaaagggcagaactgcggctatcgccggtgtcgcgcaagcggcgtgctgacgaccagctgcggcaataccctcacatgttacctgaaggccactgcagcctgtcgagctgcgaagctccaggactgcacaatgcttgtgtgtggggacgaccttgtcgtyatctgtgagagcgcggggacccaagaggacgcagcgagcctac

SEQ ID NO: 214 - DNA, 329 bp, Hepatitis C virus
ggtcactgagaatgacatycgtgttgaggagtcaatttaccaatgttgtgacttggcycccgaagccagacaggycataaggtcgctcacagagcggctttayatcgggggtccyctaaccaattcaaaagggcaaaactgcggttatcgccggtgtcgcgcragcggcgtgctgacgactagctgcggcaayacccttacatgttacttgaargcctctgcrgcctgtcgagctgcgaagctccaggactgcacgatgctcgtgtgcggagacgacctcgtcgttatctgtgagagcgcggggacccacgaggatgcggcgagcctac

SEQ ID NO: 215 - DNA, 543 bp, Hepatitis C virus
gcgcctatcacggcctacgcccarcaaacacggggcttgtttggctgtatcatcactagcctcacaggccgggacaagaaccaggtcgagggggaggtccaagtggtttccaccgccacacaatctttcctggcgacctgtgtcaacggtgtktgttggactgtcttccacggcgccggttcaaagaccctggctggcccaaagggyccaatcacccaaatgtacaccaatgtagaccaggacctcgtcggctggccggcccccccyggggcgcgctctctracaccatgcacctgtggcagctcggacctttacttggtcacgaggcatgctgatgttatcccggtgcgccggcggggcgacagtaggggragcctactctcccccaggcctgtgtcctacttaaaaggctcttcgggtggwccrctgctctgcccctcggggcacgctgtgggcgtcttccgggctgctgtgtgcacccggggggtcgcgaaggcggtggactttgtacccgtagagtctatggagactaccatgcggtcc

SEQ ID NO: 216 - DNA, 543 bp, Hepatitis C virus
gcgcccatcacggcctacgcccaacaracgaggggcctacttggctgtatcatcaccagcctcacaggccgggacaagaaccaggtygagggggaggttcaggtggtctccactgcaacacagtccttcctggcracttgcatcaacggcgtgtgttggactgtctttcatggagccggctctaagaccctagccggcccaaaggggccgatcacccagatgtacaccaatgtagaccaggacctcgtcggctggcaagcgcccccyggggcgcgttccttgacaccgtgcacctgcggcagctcggacctttacttggtcacgaggcatgccgatgtcattccggtgcgccggcgaggtgacagcagggggagcttgctctccccccggcccatttcytacttraaaggctcttcgggtggtccrytgctctgcccctcggggcacgcygtgggcatcttccgggctgccgtgtgcacycggggggttgccaaggcrgtggattttgtacccgttgagtctatggaaactacyatgcggtcc

SEQ ID NO: 217 - DNA, 543 bp, Hepatitis C virus
gcgcctattacggcctacgcccaacagacgaggggcctattaggctgcatcatcactagcctcacaggccgagacaagaaccaggtcgagggggaggttcaggtggtttctaccgcaacacaatccttcctagcgacttgcgtcaacggcgtgtgttggactgtctatcatggcgccggctctaagaccttagccggcccaaaggggcctgtcacccaaatgtacaccaatgtagaccaagacctcgtcggctggccagcgccccccggggcgcgttccttgacaccatgtacttgcggcagttcggacctttacttggtcacgagacatgccgatgtcattccggtgcgccggcggggcgacagcagggggagcctgctctcccccaggcctgtctcctatttgaagggctcttcgggtggtccactgctctgcccttcagggcacgccgtgggcatcttccgggctgccgtgtgcacccgaggggttgccaaggcggtggactttgtgcccgtcgagtccatggaaactactatgcggtct

SEQ ID NO: 218 - DNA, 543 bp, Hepatitis C virus
gcgcctatcacggcttactcccaacagacgcggggcctgcttggctgcatcatcacyagcctcacaggcagrgacaagaaccaggtcgagggggaagtccaagtggtttccaccgcaacacaatcttttctagcgacctgtgtcaacggcgtgtgttggactgttttccatggcgccggctcaaaaaccttagccggcccaaagggcccggtcacccaaatgtacaccaatgtagaccaggacctcgtcggctggcaggcgcctaccggggcgcgttctttaacaccatgcacctgcggcagctcggacctttatttggtcacgaggcatgctgatgtcattccggtgcgccggcggggcgacagccgggggagtctactctcccccaggcccgtctcctacttgaagggctcctcgggtggtccgctgctctgcccctcggggcatgcagtgggcatcttccgggctgccgtgtgcacccggggggtcgcaaaggcagtggacttcatacccgttgagtctatggaaactactatgcggtcc

SEQ ID NO: 219 - DNA, 543 bp, Hepatitis C virus
gcgcctatcacagcctactcccaacagacgcggggcctgcttggctgcatcatcactagcctcacaggccgggacaagaaccaggtcgagggggaggttcaagtggtttccaccgcgacacaatctttcctggcgacctgcgtcaacggcgtgtgttggactgtctaccatggtgccggctcgaagaccctagccggcccaaagggcccgatcacccaaatgtacaccaatgtagaccaggacctcgtcggctggccggcgccctccggagcgcgctccttgacaccgtgcacctgcggcagctcagacctytacttggtcacgaggcatgctgatgttgttccggtgcgccggcggggcgacagcaggggaagcctactctcccccaggcccatttcctacttgaagggctcttcgggtggcccgctgctttgcccctcggggcacgcggtgggcatcttccgggctgctgtatgcacccggggggtcgcgaaggcggtggactttgtacccgttgagtctatggaaaccaccatgcggtct

SEQ ID NO: 220 - DNA, 543 bp, Hepatitis C virus
gcgcctattacggcctactcccaacagacgcgaggcctacttggctgcatcatcactagcctcacaggccgggacaggaaccaggtcgagggggaggtccaagtggtctccaccgcaacacaatctttcctggcgacctgcgtcaatggcgtgtgttggactgtctatcatggtgccggctcaaagacccttgccggcccaaagggcccaatcacccaaatgtacaccaatgtggaccaggacctcgtcggctggcaagcgccccccggggcgcgttccttgacaccatgcacctgcggcagctcggacctttacttggtcacgaggcatgccgatgtcattccggtgcgccggcggggcgacagcagggggagcctactctcccccaggcccgtctcctacttgaagggctcttcgggcggtccactgctctgcccctcggggcacgctgtgggcatctttcgggctgccgtgtgcacccgaggggttgcgaaggcggtggactttgtacccgtcgagtctatggaaaccactatgcggtcc

SEQ ID NO: 221 - protein, 181 aa, Hepatitis C virus
APITAYSQQTRGLLGCIITSLTGRDRNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRS

SEQ ID NO: 222 - protein, 181 aa, Hepatitis C virus
  MOD_RES (104)..(104): Any amino acid
APITAYSQQTRGLLGCIITSLTGRDKNQVEGEVQVVSTATQSFLATCVNGVCWTVYHGAGSKTLAGPKGPITQMYTNVDQDLVGWPAPSGARSLTPCTCGSSDXYLVTRHADVVPVRRRGDSRGSLLSPRPISYLKGSSGGPLLCPSGHAVGIFRAAVCTRGVAKAVDFVPVESMETTMRS

SEQ ID NO: 223 - DNA, 1776 bp, Hepatitis C virus
tcgatgtcctacacgtggacgggcgccctgatcacgccgtgcgccgcggaggaaagcaagctgcctatcaatgcattgagcaactcactgctgcgtcaccacaatatggtttatgctacaacatcccgcagcgcaagccagcggcagaagaaggtcacttttgacagactgcaagtcctggacgaccactaccgggacgtgctcaaggagatgaaggcgaaggcgtccacagttaaggctaagcttctatctgtagaggaagcctgtaaactgacgcccccacattcggccagatccaaatttggctayggggcaaaggacgtccggaacctatccagcaaggccgttaaccacatccgctccgtgtggaaggacttgctggaagacactgagacaccaattgacaccaccatcatggcaaaaaacgaggtyttctgcgtccaaccagagaaaggaggccgcaagccagctcgccttatcgtgttcccagacttgggagttcgtgtgtgcgagaaaatggccctttacgacgtggtctccactcttcctcaagccgtgatgggctcctcatatggattccagtactctcctggacagcgggttgaattcctggtgaatgcctggaagtcgaagaagaaccctatgggcttcgcatatgacacccgctgttttgactcaacagtcactgagagtgacatccgcgttgaggagtcaatctaccaatgttgtgacttggcccccgaagccaaacaggccataaagtcgctcacagagcggctttacatcgggggtcccctgactaattcaaaagggcagaactgcggctatcgccggtgccgcgccagcggcgtactgacgaccagctgtggtaataccctcacatgttacttgaaagcctctgcggcctgtcgagctgcaaagctccaggactgcacgatgctcgtgtgcggagacgaccttgtcgttatctgtgagagcgcgggaacccaggaggacgcggcgagcctacgagtcttcacggaggctatgactaggtactccgccccccccggggacccgccccagccagagtacgacttggagttgataacatcatgctcctccaacgtgtcggtcgcgcacgatgcatccggcaaacgggtgtattacctcacccgtgaccccaccacccccctcgcgagggctgcgtgggaaacagctagacacactccagttaattcttggctaggcaacatcattatgtatgcgcccaccctgtgggcaaggatgattttgatgactcacttcttctccatccttctagctcaagaacaacttgaaaaagccctggattgtcagatctacggggcctgctactccattgagccacttgacctacctcagatcattcarcgactccatggtcttagcgcattttcactccacagttactctccaggtgagatcaatagggtggcttcatgcctcaggaaacttggggtaccgcccttgcgagtctggagacatcgggccagaagtgtccgcgctaagctactgtcccagggggggagggctgccatttgtggcaagtacctcttcaactgggcrgtaaggaccaagctcaaactcactccaatcccggcagcgtcccagttggacttgtccgactggttcgttgccggctacagcgggggagacatatatcacagcctgtctcgtgcccgaccccgctggttcctgtggtgcctactcctgctttctgcgggggtaggcatctacttgctccccaaccgatga

SEQ ID NO: 224 - DNA, 1776 bp, Hepatitis C virus
tcgatgtcctacacatggacaggcgctttaatcacaccatgcgctgcggaggaaagcaagctgcccatcaacgcgctgagcaactccctgctgcgycaccacaatatggtgtatgccacaacatcccgcagcgcaagccarcggcagaagaargtcacttttgacagactgcaagtcctggacgaycattaccgggacgtrctcaaggaggtgaaggcgaaggcgtccacagttaaggcyaaacttctatccgtagaagaggcctgcaaactsacgcccccacactcagccaaatccaartttggctatggggcraaggacgtccggaacctatccagcaaggccgtyaaccacatccactccgtgtggaaggacttgctggaggacactgaaacaccaattgacactaccatcatggcaaaaaatgaggttttctgcgttcaaccggaaaagggaggccgcaagccagctcgccttatcgtgttcccagacctgggggttcgtgtgtgcgagaaaatggccctctacgacgtggtytcyacccttcctcaggccgtgatgggcccctcatacgggttccagtactctcctggacagcgggtcgagttcctggtgaatgcctggaaatcaaagaaatgccctatgggcttcgcatatgacacccgctgttttgactcaacggtcactgagagtgatatccgtactgaggagtctatttaccaatgttgtgacctggcccccgaagctagacaagtcataaggtcgctcacagagcggctttayatygggggccccctgacyaattcaaaagggcagaactgcggttatcgccggtgccgygcgagcggcgtgctgacgactagctgcggtaataccctcacatgttacttgaaggcctctgcggcctgtcgagctgcaaagctccgggactgcacgatgctcgtgtgcggagacgacctcgtcgttatctgtgaaagcgcggggacccaggaggacgcggctagcctacgagtcttcacggaggctatgactaggtactcagccccccccggggacccgccccaaccagagtacgacttggagttgataacatcatgctcctccaacgtgtcggtcgcgcacgacgcatmtggcaagagggtgtactacctcacccgtgaccccaccacccccctcgcgcgggctgcgtgggagacagctagacacactccaattaactcctggctaggcaacatcatcatgtatgcgcccacyytatgggcaaggatgattctgatgactcacttcttctccatccttctrgcycaggaacaacttgaaaaagccctagattgccaratctayggggcctgttactccattgaaccacttgacctacctcagatcattcagcgactccatggtctyagcgcattttcactccatagttactctccaggtgagatcaatagggtggcttcaagcctcaggaaacttggggtgccrcccttgcgagtctggagacatcgggccaggagygtccgcgctaagctactgtcccarggagggagggcygccacgtgtggtaagtacctcttcaactgggcagtaaggaccaagctyaaactcactccaatcccggctgcgtcccagctggacttgtccagctggttcgtygctggttacagcgggggagacatatatcacagcctgtctcgtgcccgrccccgctggttcatgtggtgcctactcctactctctgtaggggtaggcatctayctgctccccaaycgatga

SEQ ID NO: 225 - DNA, 1776 bp, Hepatitis C virus
tcgatgtcctacacatggacaggcgccctgatcacgccatgcgctgcggaggaaagcaagctgcccatcaacccgttgagcaactctttgctgcgtcaccataayatggtatacgctacaacatcccgcagcgcaagcctacggcagaagaaggtcacttttgacagactgcaagtcctggacgaccactaccgggacgtgcttaaggagatgaaggcgaaggcgtccacagttaaggctaagcttctatctgtagaagaagcctgcaaactgacacccccacactcggccagatccaaatttggctatggggcaaaggacgtccggagcctatccagcaaggccgtcaaccacatcaactccgtgtggaaggacttgctggaagacactgagacaccaattgacaccaccatcatggcaaaaaatgaggttttctgcgtccaaccagagaaaggaggccgcaagccagcccgccttatcgtgttcccagacttaggggttcgcgtgtgcgagaagatggccctttatgacgtggtctccacccttcctcaggccgtgatgggctcctcgtacggattccaatactctcctggacagcgggtcgagttcctggtgaatgcctggaaatcaaagaaatgccctatgggcttctcatatgacacccgctgttttgactcaacagtcaccgagaatgatatccgtgttgaggagtcaatttaccaatgctgtgacttggcccccgaagccaaacaggccataaggtcgctcacagagcggctttayatcgggggtcccctgactaattcaaaagggcagaactgcggttatcgccggtgccgcgcgagcggcgtgctgacgaccagctgcggtaataccctcacctgttacttgaaggccaccgcggcctgtcgagctgcaaagctccaggactgcacgatgctcgtgtgcggggacgaccttgtcgttatctgtgaaagcgcgggaacccaagaggacgcggcgaacctacgagtcttcacggaggctatgactaggtattctgccccccccggggacccgccccaaccagaatacgacttggarttgataacatcatgctcctccaacgtgtcggtcgcgcacgatgcatctggcaagcgggtgtaytacctcacccgcgaccccaccacccccctygcacgggctgcgtgggaracagctagacacactccagttaactcctggctaggcaacattatcatgtatgcgcccaccttatgggcaaggatgatcctgatgactcacttcttctccatccttctagctcaggaacaacttgaaaaagccctggattgycaaatctacggggcctgttactccattgagccacttgacctacctcagatcattcagcgactccatggccttagcgcattttcactccacagttactctccaggtgagatcaatagggtggcttcatgcctcaggaaacttggggtaccacccttgcgagtctggagacatcgggccagaagtgtccgcgctaagctactgtcccagggagggagggccgccacttgtggcaggtacctcttcaattgggcagtaaggaccaagcttaaactcactccaatcccggctgcgtcccagttggacttgtccggctggttcgttgctgggtacagcgggggagacatatatcacagcctgtctcgtgcccgaccccgctggttcctgtggtgcctactcctactttctgtaggggtaggcatctacctgctccccaaccgatga

SEQ ID NO: 226 - DNA, 1776 bp, Hepatitis C virus
tcgatgtcctayacatggacaggcgccctgatcacgccatgcgccgcggargaaagcaagctgcccatcaatgcgttgagcaactctttgctgcgtcaccataayatggtctacgccacaacatcccgcagcgcaagccagcggcagaagaaggtcacctttgacagactgcaggtcctggacgaccactaccgggacgtgcttaaggagatgaaggcgaaggcgtccacagttaaggctagacttctatcygtagaagaagcctgcaagctgacgcccccacactcagccagatccaaatttggctatggggcgaaggacgtccggaacctatctagcaaggccgttaaccacatccgctccgtgtggaaggacttgctggaagacactgaaacaccaatcgacgctaccatcatggcaaaaaatgaggttttctgcgtccaaccagagaaaggaggtcgcaagccrgctcgccttatcgtgttcccagatttgggagtccgtgtgtgcgagaaaatggccctttacgacgtggtctccacccttcctcaggccgtgatgggcccctcatacggattccaatactctcctggacagcgggtcgagttcctggtgaatgcctggaaatcaaagaaaaaccctatgggcttctcatatgacacccgctgytttgactctacggtcacygagagygacatccgtactgaggagtcaatttaccaatgttgtgacttggcccccgaagccagacaggttataaggtcgctcacagagcggctttatatcgggggtcctytgactaattcaaaagggcagaactgcggctatcgccggtgtcgcgcaagcggcgtgctgacgaccagctgcggcaataccctcacatgttacctgaaggccactgcagcctgtcgagctgcgaagctccaggactgcacaatgcttgtgtgtggggacgaccttgtcgtyatctgtgagagcgcggggacccaagaggacgcagcgagcctacgagtcttcacggaggctatgactaggtactctgctccccccggggacccgccccggccggaatacgacttggarttaataacatcatgctcctccaacgtgtcggtcgcgcacgacgcacayggcaaaagggtgtactacctcacccgtgaccccaccaccccccttgcgcgggcygcatgggagacagctagacacactccagtcaactcctggctaggcaacatcatcatgtatgcgcccaccttgtgggcaaggatgatyctgatgacycatttcttctccatccttctagcccaggagcaacttgaaaaagccctagattgtcagatctacggggcctgttactccattgagccacttgacctacctcagatcattcagcgactccatggtcttagcgcattttcactccacagttactctccaggtgagatcaatagggtggcttcatgcctcaggaaacttggggtaccacccctgcgagtctggagacatcgggccagaagtgtccgcgctaagctgctgtcccggggggggagggctgccacttgtggcaagtacctcttcaactgggcrgtaaggaccaagctcaaactcactccaatcccggctgcgttcaagctggacttgtccggctggttcgttgctggttacagcgggggagacatatatcacagcctgtctcgtgcccgaccccgctggttyrtgtggtgcctactcctactttctgtaggggtaggcatctacctgctccccaaccgatga

SEQ ID NO: 227 - DNA, 1776 bp, Hepatitis C virus
tcgatgtcctacacatggacaggcgccttgatcacaccgtgcgctgcggargagagcaagctgcccatcaaygcgctgagcaactctttgytgcgycaccataacatgrtctatgccacaacatcccgcagcgcyagccaamggcagargaaggtcacttttgayagactgcargtcctggacgaccactaccgggacgtgctyaaggagatgaaggcgaaggcgtccacagtcaaggctaaacttctatccgtagargaagcctgyaagctgacrcccccacactcggccagatcyaaatttggctatggggcaaaggacgtccggaacctatccagcaaggccgttaaccacatccactccgtgtggaaggacttgctggaagacactgacacaccaattgacaccaccatcatggcaaaaaatgaggttttctgyatccaaccagagaaaggaggccgcaagccagctcgccttatcgtrtacccagacctgggggtccgrgtgtgcgagaagatggctctttaygatgtggtctccacycttcctcaggccgtgatgggcccctcrtacggatttcagtactctcctggacagcgggttgagttcctggtgaawgcctggaartcaaagaaatgccctatgggcttcgcrtatgacacccgctgcttygactcracggtcactgagaatgacatycgtgttgaggagtcaatttaccaatgttgtgacttggcycccgaagccagacaggycataaggtcgctcacagagcggctttayatcgggggtccyctaaccaattcaaaagggcaaaactgcggttatcgccggtgtcgcgcragcggcgtgctgacgactagctgcggcaayacccttacatgttacttgaargcctctgcrgcctgtcgagctgcgaagctccaggactgcacgatgctcgtgtgcggagacgacctcgtcgttatctgtgagagcgcggggacccacgaggatgcggcgagcctacgagtcttyacggaggctatgactaggtactccgcccccccyggggacccgcctcagccagaatacgacttagagctgataacatcatgctcttccaaygtgtcrgtcgcgcacgatgcatcyggcaaaagggtrtactacctcacccgtgaccccaccaccccccttgcrcgggctgcgtgggaracagctagacacactccagtyaactcctggctaggcaacatcatcatgtaygcgcccaccytatgggcaaggatgatcctgatgactcatttcttctccatccttctagctcaggagcaacttgaaaaagccctagattgtcagatctayggggcctgttactccattgaaccacttgacctacctcaaatcattcarcgactccatggtattagcgcgttttcactccayagttactctccaggwgagatcaatagggtggcttcatgcctcaggaaacttggggtaccrcccttgcgagtctggagacatcgggccaggagtgtccgcgctaagytactgtcccagggggggagggctgccacttgtggcaartacctcttcaactgggcagtaaraaccaagcttaatctcactccaattccggctgcgtccaagctggatttatccrgctggttcgttgccggytacagcgggggagacatatatcacagcgtgtctcmtgcccgaccccgctggttcatgtggtgcctrctcctactktctgtaggrgtaggcatctacctgctycccaaccgatga

SEQ ID NO: 228 - DNA, 1776 bp, Hepatitis C virus
tcgatgtcctacacatggacaggcgccctgatcacgccatgcgctgcggaggaaaccaagctgcccatcaatgcactgagcaactctttgctccgtcaccacaacttggtctatgctacaacatctcgcagcgcaagcctgcggcagaagaaggtcacctttgacagactgcaggtcctggacgaccactaccgggacgtgctcaaggagatgaaggcgaaggcgtccacagttaaggctaaacttctatccgtggaggaagcctgtaagctgacgcccccacattcggccagatctaaatttggctatggggcaaaggacgtccggaacctatccagcaaggccgttaaccacatccgctccgtgtggaaggacttgctggaagacactgagacaccaattgacaccaccatcatggcaaaaaatgaggttttctgcgtccaaccagagaaggggggccgcaagccagctcgccttatcgtattcccagatttgggggttcgtgtgtgcgagaaaatggccctttacgatgtggtctccaccctccctcaggccgtgatgggctcttcatacggattccaatactctcctggacagcgggtcgagttcctggtgaatgcctggaaagcgaagaaatgccctatgggcttcgcatatgacacccgctgttttgactcaacggtcactgagaatgacatccgtgttgaggagtcaatctaccaatgttgtgacttggcccccgaagccagacaggccataaggtcgctcacagagcggctttacatcgggggccccctgactaattctaaagggcagaactgcggctatcgccggtgccgcgcgagcggtgtactgacgaccagctgcggtaataccctcacatgttacttgaaggccgctgcggcctgtcgagctgcgaagctccaggactgcacgatgctcgtatgcggagacgaccttgtcgttatctgtgaaagcgcggggacccaagaggacgaggcgagcctacgggccttcacggaggctatgactagatactctgccccccctggggacccgcccaaaccagaatacgacttggagttgataacatcatgctcctccaatgtgtcagtcgcgcacgatgcatctggcaaaagggtgtactatctcacccgtgaccccaccaccccccttgcgcgggctgcgtgggagacagctagacacactccagtcaattcctggctaggcaacatcatcatgtatgcgcccaccttgtgggcaaggatgatcctgatgactcatttcttctccatccttctagctcaggaacaacttgaaaaagccctagattgtcagatctacggggcctgttactccattgagccacttgacctacctcagatcattcaacgactccatggccttagcgcattttcactccatagttactctccaggtgagatcaatagggtggcttcatgcctcaggaaacttggggtaccgcccttgcgagtctggagacatcgggccagaagtgtccgcgctaggctactgtcccagggggggagggctgccacttgtggcaagtacctcttcaactgggcagtaaggaccaagctcaaactcactccaatcccggctgcgtcccagttggatttatccagctggttcgttgctggttacagcgggggagacatatatcacagcctgtctcgtgcccgaccccgctggttcatgtggtgcctactcctactttctgtaggggtaggcatctatctactccccaaccgatga